(19) United States
(12) Patent Application Publication
(10) Pub. No.: US 2025/0066806 A1
(43) Pub. Date: Feb. 27, 2025

(54) COMPOSITIONS AND METHODS COMPRISING PLANTS WITH MODIFIED SUGAR CONTENT

(71) Applicant: Benson Hill, Inc., St. Louis, MO (US)

(72) Inventors: Matthew Begemann, St. Louis, MO (US); Emma January, St. Louis, MO (US)

(21) Appl. No.: 18/703,129

(22) PCT Filed: Oct. 21, 2022
(86) PCT No.: PCT/IB2022/060152

§ 371 Date: Apr. 19, 2024

Related U.S. Application Data
(60) Provisional application No. 63/270,159, filed on Oct. 21, 2021.

(51) Int. Cl. C12N 15/82; C07K 14/415; C12N 9/22; C12N 15/11

(52) U.S. Cl. CPC C12N 15/8246; C07K 14/415; C12N 9/22; C12N 15/111; C12N 15/8213; C12N 2310/20

(57) ABSTRACT

Provided herein are plants and plant parts (e.g., tomato plants and plant parts) comprising altered expression or function of the bZIP transcription factor, plants and plant parts comprising one or more mutations in the upstream regulatory region of the bZIP transcription factor gene (e.g., bZIP SIRT element), and plants and plant parts having increased sugar and/or total soluble solids levels. Also disclosed herein are methods of producing such plants and plant parts.

### RELATED APPLICATIONS

This application claims priority to U.S. Provisional Application No. 63/270,159, filed on Oct. 21, 2021, the content of which is incorporated herein by reference in its entirety.

### SEQUENCE LISTING

This application contains a Sequence Listing which is submitted herewith in electronically readable format. The Sequence Listing file was created on Oct. 20, 2022, is named “B88552_1310WO_SL.xml” and its size is 53.6 kb. The entire contents of the Sequence Listing in the sequencelisting.xml file are incorporated by reference herein.

### FIELD OF THE INVENTION

The present disclosure relates to plants and plant parts having increased sugar and/or total soluble solids content, comprising one or more mutations in the upstream open reading frame (uORF) of the bZIP transcription factor gene, and associated methods and compositions thereof.

### BACKGROUND OF THE INVENTION

Sugar is a primary driver of flavor in fruits and vegetables. Sweetness and enhanced sugar content are desirable traits and impact the overall flavor acceptance of fruits and vegetables by consumers. Sugar content in fruits can be modified by manipulating carbohydrate metabolism or carbohydrate transport in the plants.

A sucrose-sensitive upstream Open Reading Frame (ORF), termed “sucrose-induced repression of translation (SIRT)”, can repress the downstream ORF in plants in the presence of sucrose. Basic region leucine zipper (bZIP) family transcription factor homologs have been identified in fruits and vegetables, containing an upstream SIRT element. The bZIP transcription factors can regulate expression of genes involved in carbohydrate metabolism or transport, including asparagine synthetase and proline dehydrogenase.

Accordingly, modulating the function of the SIRT element upstream of bZIP transcription factor may be useful in modulating the sugar content of fruits and vegetables to provide a product with increased sweetness that could have important commercial advantages.

### SUMMARY OF THE INVENTION

The present disclosure provides plants and plant parts comprising altered function of the bZIP transcription factor, plants and plant parts comprising one or more mutations in the upstream regulatory region of the bZIP transcription factor gene (e.g., SlbZIP SIRT element), and plants and plant parts having increased sugar content and/or increased total soluble solids levels. Also disclosed herein are methods for producing such plants and plant parts.

In some aspects, the present disclosure provides a plant or plant part comprising altered expression or function of a basic region/leucine zipper motif (bZIP) transcription factor gene or a homolog thereof, wherein said plant or plant part comprises one or more insertions, substitutions, or deletions in an upstream open reading frame (uORF) of the bZIP transcription factor gene or the homolog thereof, wherein the uORF comprises a sucrose induced repression of translation (SIRT) element; wherein the one or more insertions, substitutions, or deletions alter function of the SIRT element; and wherein sugar content of said plant or plant part is increased relative to a control plant or plant part.

In some embodiments, the SIRT element within the uORF of the bZIP transcription factor gene or homolog thereof in which the one or more insertions, substitutions, or deletions are located comprises a nucleic acid molecule: (a) comprising a nucleic acid sequence encoding a polypeptide comprising an amino acid sequence having at least 90% sequence identity to the amino acid sequence set forth in SEQ ID NO: No: 5 or 6, wherein the SIRT element retains SIRT function; (b) comprising a nucleic acid sequence encoding a polypeptide comprising the amino acid sequence set forth in SEQ ID NO: 5 or 6; (c) comprising a nucleic acid sequence having at least 90% sequence identity to the nucleic acid sequence set forth in SEQ ID NO: 3 or 4, wherein the SIRT element retains SIRT function; and/or (d) comprising the nucleic acid sequence set forth in SEQ ID NO: 3 or 4.

In some embodiments, the plant or plant part of the present disclosure comprises a deletion of 7-9 nucleotides in the nucleic acid sequence encoding the SIRT element. In some embodiments, the nucleic acid sequence encoding SIRT element comprising the deletion: (a) comprises a nucleic acid sequence that shares at least 90% identity with a nucleic acid sequence selected from the group consisting of SEQ ID NOs: 7-10; or (b) comprises a nucleic acid sequence selected from the group consisting of SEQ ID NOs: 7-10.

In some embodiments, expression or function of a bZIP transcription factor gene in the plant or plant part of the present disclosure is increased as compared to the control plant or plant part. In some embodiments, in the plant or plant part of the present disclosure, expression or function of a molecule regulated by the bZIP transcription factor is modulated, wherein the modulation comprises: (a) an increased baseline expression or function in the absence of sucrose relative to the control plant or plant part; (b) decreased suppression of the expression or the function in the presence of sucrose relative to the control plant or plant part; and/or (c) an increase in the expression or the function in the presence of sucrose relative to in the absence of sucrose. In some embodiments, in the plant or plant part of the present disclosure, the molecule regulated by the bZIP transcription factor comprises from about 1.1-fold to about 25-fold increase (resulting in levels of about 110% to about 2500%) in the baseline expression or function relative to the control plant or plant part. In some embodiments, the molecule regulated by the bZIP transcription factor comprises from about 1.1-fold to about 10-fold increase (resulting in levels of about 110% to about 1000%) in the expression or the function in the presence of sucrose relative to in the absence of sucrose. In some embodiments, the molecule regulated by the bZIP transcription factor is one or more of asparagine synthase (ASN), proline dehydrogenase (PDH), sucrose phosphate synthase (SPS), sucrose-6′-phosphate phosphatase (SPP), and calmodulin.

In some embodiments, in the plant or plant part of the present disclosure, a level of total soluble solids (TSS) is increased relative to a control plant or plant part. In some embodiments, sugar content of the plant or plant part is increased relative to a control plant or plant part, wherein the sugar is one or more of total sugar, sucrose, glucose, fructose, galactose, maltose, and lactose.

In some embodiments, the bZIP transcription factor gene of the plant or plant part of the present disclosure encodes an SlbZIP1 transcription factor or an SlbZIP2 transcription factor. In some embodiments, said SlbZIP1 transcription factor: (a) comprises a nucleic acid sequence having at least 80% sequence identity to SEQ ID NO: 21; or (b) comprises the nucleic acid sequence set forth in SEQ ID NO: 21; and wherein said SlbZIP2 transcription factor: (c) comprises a nucleic acid sequence having at least 80% sequence identity to SEQ ID NO: 22; or (d) comprises the nucleic acid sequence set forth in SEQ ID NO: 22.

In some embodiments, the plant or plant part provided herein is a tomato plant or plant part.

In some aspects, the present disclosure provides a method for altering expression or function of a basic region/leucine zipper motif (bZIP) transcription factor gene or a homolog thereof in a plant or plant part, said method comprising introducing one or more insertions, substitutions, or deletions into the upstream open reading frame (uORF) of the bZIP transcription factor gene or the homolog thereof in the plant or plant part, wherein the uORF comprises a sucrose induced repression of translation (SIRT) element; wherein function of the SIRT element is altered; and wherein sugar content of said plant or plant part is increased relative to a control plant or plant part.

In some embodiments, the SIRT element within the uORF of the bZIP transcription factor gene or homolog thereof to which the one or more insertions, substitutions, or deletions are introduced comprises a nucleic acid molecule: (a) comprising a nucleic acid sequence encoding a polypeptide comprising an amino acid sequence having at least 90% sequence identity to the amino acid sequence set forth in SEQ ID NO: No: 5 or 6, wherein the SIRT element retains SIRT function; (b) comprising a nucleic acid sequence encoding a polypeptide comprising the amino acid sequence set forth in SEQ ID NO: 5 or 6; (c) comprising a nucleic acid sequence having at least 90% sequence identity to the nucleic acid sequence set forth in SEQ ID NO: No: 3 or 4 and encoding a SIRT element, wherein the SIRT element retains SIRT function; and/or (d) comprising the nucleic acid sequence set forth in SEQ ID NO: 3 or 4.

In some embodiments, the one or more insertions, substitutions, or deletions of the method of the present disclosure comprise a deletion of 7-9 nucleotides in the nucleic acid sequence encoding the SIRT element. In some embodiments, the nucleic acid sequence encoding the SIRT element comprising the deletion is encoded by a nucleic acid sequence: (a) comprising a nucleic acid sequence that shares at least 90% identity with a nucleic acid sequence selected from the group consisting of SEQ ID NOs: 7-10; or (b) comprising a nucleic acid sequence selected from the group consisting of SEQ ID NOs: 7-10.

In some embodiments, the method of the present disclosure comprises introducing a gene editing system into the plant or plant part. In some embodiments, the gene editing system is one or more of a zinc finger nuclease system, a transcription activator-like effector nucleases (TALEN) system, and a clustered regularly interspaced short palindromic repeats (CRISPR) system. In some embodiments, the gene editing system comprises a CRISPR nuclease. In some embodiments, the CRISPR nuclease is a Cas12a nuclease. In some embodiments, the Cas12a nuclease is McCpf1.

In some embodiments, the gene editing system comprises one or more guide RNAs (gRNAs). In some embodiments, the one or more gRNAs comprise a nucleic acid sequence complementary to a region of a nucleic acid sequence encoding a SIRT element, e.g., the SIRT element of the SlbZIP1 transcription factor gene, or the SIRT element of the SlbZIP2 transcription factor gene. In some embodiments, at least one of the gRNAs is encoded by a nucleic acid sequence: (a) comprising a nucleic acid sequence that shares at least 80% sequence identity with the nucleic acid sequence selected from the group consisting of SEQ ID NOs: 12-19; or (b) comprising a nucleic acid sequence selected from the group consisting of SEQ ID NOs: 12-19.

In some embodiments according to the method of the present disclosure, expression or function of the bZIP transcription factor is increased as compared to the control plant or plant part. In some embodiments, expression or function of a molecule regulated by the bZIP transcription factor is modulated, wherein the modulation comprises: (a) an increased baseline expression or function in the absence of sucrose relative to the control plant or plant part; (b) decreased suppression of the expression or the function in the presence of sucrose relative to the control plant or plant part; and/or (c) an increase in the expression or the function in the presence of sucrose relative to in the absence of sucrose. In some embodiments according to the methods of the present disclosure, the molecule regulated by the bZIP transcription factor comprises from about 1.1-fold to about 25-fold increase (resulting in levels of about 110% to about 2500%) in the baseline expression or function relative to the control plant or plant part. In some embodiments, the molecule regulated by the bZIP transcription factor comprises from about 1.1-fold to about 10-fold increase (resulting in levels of about 110% to about 1000%) in the expression or the function in the presence of sucrose relative to in the absence of sucrose. In some embodiments, the molecule is asparagine synthase (ASN), proline dehydrogenase (PDH), sucrose phosphate synthase (SPS), sucrose-6′-phosphate phosphatase (SPP), or calmodulin.

In some embodiments according to the method of the present disclosure, a level of total soluble solids (TSS) is increased in the plant or plant part relative to a control plant or plant part. In some embodiments, sugar content of the plant or plant part is increased relative to a control plant or plant part, wherein the sugar is one or more of total sugar, sucrose, glucose, fructose, galactose, maltose, and lactose.

In some embodiments of the methods according to the present disclosure, the bZIP transcription factor gene encodes an SlbZIP1 transcription factor or an SlbZIP2 transcription factor. In some embodiments, said SlbZIP1 transcription factor: (a) comprises a nucleic acid sequence having at least 80% sequence identity to SEQ ID NO: 21; or (b) comprises the nucleic acid sequence set forth in SEQ ID NO: 21; and wherein said SlbZIP2 transcription factor: (c) comprises a nucleic acid sequence having at least 80% sequence identity to SEQ ID NO: 22; or (d) comprises the nucleic acid sequence set forth in SEQ ID NO: 22.

In some embodiments, the plant or plant part of the methods provided herein is a tomato plant or plant part.

In some aspects, the present disclosure provides a nucleic acid molecule comprising a nucleic acid sequence encoding a mutated SIRT element, comprising one or more insertions, substitutions, or deletions compared to the corresponding native nucleic acid sequence encoding a SIRT element, wherein said nucleic acid sequence encoding the mutated SIRT element: (a) comprises a nucleic acid sequence that shares at least 90% identity with a nucleic acid sequence selected from the group consisting of SEQ ID NOs: 7-10; or (b) comprises a nucleic acid sequence selected from the group consisting of SEQ ID NOs: 7-10.

In some embodiments, in the nucleic acid molecule according to the present disclosure, said nucleic acid sequence encoding a mutated SIRT element is operably linked to a nucleic acid sequence encoding a bZIP transcription factor. In some embodiments, the bZIP transcription factor gene encodes an SlbZIP1 transcription factor or an SlbZIP2 transcription factor. In some embodiments, in the nucleic acid molecule according to the present disclosure, said SlbZIP1 transcription factor gene: (a) comprises a nucleic acid sequence having at least 80% sequence identity to SEQ ID NO: 21; or (b) comprises the nucleic acid sequence set forth in SEQ ID NO: 21; and wherein said SlbZIP2 transcription factor gene: (c) comprises a nucleic acid sequence having at least 80% sequence identity to SEQ ID NO: 22; or (d) comprises the nucleic acid sequence set forth in SEQ ID NO: 22.

In some aspects, the present disclosure provides a DNA construct comprising, in operable linkage: (a) a promoter that is functional in a plant cell; and (b) the nucleic acid molecule of the present disclosure comprising a nucleic acid sequence encoding a mutated SIRT element.

In some aspects, the present disclosure provides a cell comprising the nucleic acid molecule of the present disclosure or the DNA construct of the present disclosure.

### BRIEF DESCRIPTION OF THE DRAWINGS

FIG. 1 schematically depicts a construct comprising coding sequences for a promoter, a sucrose-induced repression of translation (SIRT) element, a basic region leucine zipper (bZIP) transcription factor, and a terminator. SIRT represses bZIP transcription factor in the presence of sucrose, thereby affecting expression and/or function of genes downstream of bZIP transcription factor that are involved in sucrose metabolism, including asparagine synthase (ASN), proline dehydrogenase (PDH), sucrose phosphate synthase (SPS), and sucrose-6′-phosphate phosphatase (SPP). In some aspects of the present disclosure, alteration of the SIRT function results in sucrose metabolism reprogramming in a plant or plant part.

FIG. 2 depicts 5′ untranslated region (5′UTR) of the tomato (Solanum lycopersicum) bZIP1 (SlbZIP1) transcription factor gene including the SIRT element and four exemplary guide RNAs (gRNAs) targeting the SlbZIP1 SIRT element (bZIP1 gRNA1, bZIP1 gRNA2, bZIP1 gRNA3, and bZIP1 gRNA4). The nucleic acid sequences for targeting regions of bZIP1 gRNA1-4 are set forth as SEQ ID NOs: 12-15. The nucleic acid sequence of SlbZIP1 SIRT element is set forth as SEQ ID NO: 3. The amino acid sequence of SlbZIP1 SIRT element is set forth as SEQ ID NO: 5.

FIG. 3 is a graph of editing efficiency (%) of the SlbZIP1 uORF including the SIRT element using bZIP1 gRNA1-4 in tomato protoplasts, where each gRNA cassette and a Gen6 McCpf1 nuclease were introduced into the tomato protoplasts.

FIG. 4 depicts 5′ untranslated region (5′UTR) of the tomato (Solanum lycopersicum) bZIP2 (SlbZIP2) transcription factor gene including the SIRT element and four exemplary guide RNAs (gRNAs) targeting the SlbZIP2 SIRT element (bZIP2 gRNA1, bZIP2 gRNA2, bZIP2 gRNA3, and bZIP2 gRNA4). The nucleic acid sequences for targeting regions of bZIP2 gRNA1-4 are set forth as SEQ ID NOs: 16-19. The nucleic acid sequence of SlbZIP2 SIRT element is set forth as SEQ ID NO: 4. The amino acid sequence of SlbZIP1 SIRT element is set forth as SEQ ID NO: 6.

FIG. 5 is a graph of editing efficiency (%) of the SlbZIP2 uORF including the SIRT element using bZIP2 gRNA1-4 in tomato protoplasts, where each gRNA cassette and a Gen6 McCpf1 nuclease were introduced into tomato protoplasts (“bZIP2-gRNA1”, “bZIP2-gRNA2”, “bZIP2-gRNA3”, “bZIP2-gRNA4”). The editing efficiency of the polygalacturonase-2 (PG2) gene using a gRNA and a Gen6 McCpf1 nuclease in tomato protoplasts is shown as a control (“PG2-gRNA4”).

FIG. 6 depicts an exemplary sequence of part of a wild-type SlbZIP1 uORF including a SIRT element (top row; SEQ ID NO: 3) and exemplary sequences of part of a mutated SlbZIP uORF including a mutated SIRT element (rows 2-4). The mutations were introduced in tomato protoplasts using gRNA4 and a Gen6 McCPF1 nuclease.

FIG. 7 depicts constructs each comprising a 2×35s promoter, a wild-type SIRT (WT-SIRT) or a mutated SIRT (Mut-SIRT), a GFP coding sequence, and a NOSt terminator. In Mut-SIRT, the first, fourth, and tenth amino acid of the SIRT element (methionine) have been substituted with leucine (i.e., M1L, M4L, and M10L). The amino sequences of WT-SIRT and Mut-SIRT are set forth as SEQ ID NOs: 5 and 11, respectively. FIG. 7 also depicts a graph of GFP expression (pg/mL) in the absence (−) or presence (+) of sucrose in tobacco leaves infiltrated with the WT-SIRT or the Mut-SIRT construct.

FIG. 8 depicts constructs each comprising a wild-type SIRT (WT-SIRT) or mutated SIRT (4661 Mut-SIRT), and a GFP coding sequence. The 4661 Mut-SIRT comprises an 8 bp deletion compared to the WT-SIRT. The nucleic acid sequences of WT-SIRT and 4661 Mut-SIRT are set forth as SEQ ID NOs: 3 and 7, respectively. FIG. 8 also depicts a graph of GFP expression (pg/mL) in the absence (−) or presence (+) of sucrose in tobacco leaves infiltrated with the WT-SIRT or 4661 Mut-SIRT construct.

FIG. 9 depicts constructs each comprising a wild-type SIRT (WT-SIRT) or mutated SIRT (4663 Mut-SIRT), and a GFP coding sequence. The 4663 Mut-SIRT comprises an 8 bp deletion compared to the WT-SIRT. The nucleic acid sequence of WT-SIRT and 4663 Mut-SIRT are set forth as SEQ ID NOs: 3 and 8, respectively. FIG. 9 also depicts a graph of GFP expression (pg/mL) in the absence (−) or presence (+) of sucrose in tobacco leaves infiltrated with the WT-SIRT or 4663 Mut-SIRT construct.

FIG. 10 depicts constructs each comprising a wild-type SIRT (WT-SIRT) or mutated SIRT (4664 Mut-SIRT), and a GFP coding sequence. The 4664 Mut-SIRT comprises an 9 bp deletion compared to the WT-SIRT. The nucleic acid sequence of WT-SIRT and 4664 Mut-SIRT are set forth as SEQ ID NOs: 3 and 9, respectively. FIG. 10 also depicts a graph of GFP expression (pg/mL) in the absence (−) or presence (+) of sucrose in tobacco leaves infiltrated with the WT-SIRT or 4664 Mut-SIRT construct.

FIG. 11 depicts constructs each comprising a wild-type SIRT (WT-SIRT) or mutated SIRT (4665 Mut-SIRT), and a GFP coding sequence. The SlbZIP1 uORF comprising the 4665 Mut-SIRT comprises an 8 bp deletion compared to the wild-type SlbZIP1 uORF, with the 4665 Mut-SIRT comprising a 7 bp deletion compared to the WT-SIRT. The nucleic acid sequence of WT-SIRT and 4665 Mut-SIRT are set forth as SEQ ID NOs: 3 and 10, respectively. FIG. 11 also depicts a graph of GFP expression (pg/mL) in the absence (−) or presence (+) of sucrose in tobacco leaves infiltrated with the WT-SIRT or 4665 Mut-SIRT construct.

FIG. 12 depicts a multiple sequence alignment of the amino acid sequences of bZIP SIRT elements from the model species Arabidopsis thaliana and multiple crop species (Solanum lycopersicum, Capsicum annuum, and Solanum melongena) showing high conservation of the C-terminal end (3′) of the amino acid sequence. Alignment was completed using the MUSCLE align program on the Molecular Evolutionary Genetics Analysis-X (MEGA-X) software. Conserved amino acids are denoted by a “*” in the top row. Amino acids of the same functional group are color-coded similarly.

### DETAILED DESCRIPTION OF THE INVENTION

The present disclosure now will be described more fully hereinafter. The disclosure may be embodied in many different forms and should not be construed as limited to the aspects set forth herein; rather, these aspects are provided so that this disclosure will satisfy applicable legal requirements.

### I. Definitions

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

As used herein, “a,” “an,” or “the” can mean one or more than one. For example, “a” cell can mean a single cell or a multiplicity of cells. Further, the term “a plant” may include a plurality of plants.

As used herein, unless specifically indicated otherwise, the word “or” is used in the inclusive sense of “and/or” and not the exclusive sense of “either/or.”

The term “about” or “approximately” usually means within 5%, or more preferably within 1%, of a given value or range.

The terms “comprises”, “comprising”, “includes”, “including”, “having” and their conjugates mean “including but not limited to”.

Various embodiments of this disclosure may be presented in a range format. It should be noted that whenever a value or range of values of a parameter are recited, it is intended that values and ranges intermediate to the recited values are also part of this disclosure. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1-10 should be considered to have specifically disclosed subranges such as from 1 to 3, from 1 to 4, from 1 to 5, from 1 to 6, from 1 to 7, from 1 to 8, from 1 to 9, from 2 to 4, from 2 to 6, from 2 to 8, from 2 to 10, from 3 to 6, etc., as well as individual numbers within that range, for example, 1, 2, 3, 4, 5, 6, 7, 8, 9 and 10. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. The recitation of a numerical range for a variable is intended to convey that the present disclosure may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values 0 and 2 if the variable is inherently continuous.

As used herein, the terms “tomato plant”, “tomato”, “tomatoes” and the like refer to well-known plant of the Solanum genus under Solanaceae (nightshades) family, including cultivated tomato plant (e.g., Solanum lycopersicum) and wild tomato plant (e.g., Solanum chmielewskii, also known as Lycoperscicon chmielewskii). Tomato plant species include, but not limited to, Solanum lycopersicum (tomato, cherry tomato; Lycopersicon cerasiforme, Lycopersicon lycopersicum), Solanum pimpinellifolium (currant tomato; Lycopersicon esculentum ssp. intermedium, Lycopersicon esculentum ssp. pimpinellifolium, Lycopersicon esculentum var. racemigerum, Lycopersicon pissisi, Lycopersicon racemiforme, Lycopersicon racemigerum), Solanum arcanum (Lycopersicon peruvianum), Solanum chmielewskii, Solanum neorickii (Lycopersicon parviflorum), Solanum cheesmaniae (Lycopersicon peruvianum var. parviflorum), Solanum galapagense (Lycopersicon cheesmaniae), Solanum chilense (Lycopersicon atacamense, Lycopersicon bipinnatifidum, Lycopersicon peruvianum ssp. puberulum, Lycopersicon puberulum), Solanum corneliomulleri (Lycopersicon glandulosum), Solanum habrochaites (Lycopersicon agrimoniifolium, Lycopersicon hirsutum), Solanum huaylasense, Solanum peruvianum (Lycopersicon commutatum, Lycopersicon dentatum, Lycopersicon regulare), Solanum pennellii, Physalis angulata, Solanum carolinense, Solanum quadriloculatum, and Solanum wallacei. Tomato plants can include any other species or variety of tomato not listed herewith.

A plant refers to a whole plant, any part thereof, or a cell or tissue culture derived from a plant, comprising any of: whole plants, plant components or organs (e.g., leaves, stems, roots, embryos, pollen, ovules, seeds, leaves, flowers, branches, fruit, pulp, juice, kernels, ears, cobs, husks, stalks, root tips, anthers, etc.), plant tissues, seeds, plant cells, protoplasts and/or progeny of the same. A plant cell is a biological cell of a plant, taken from a plant or derived through culture of a cell taken from a plant. Grain is intended to mean the mature seed produced by commercial growers for purposes other than growing or reproducing the species. Progeny, variants, and mutants of the regenerated plants are also included within the scope of the invention.

As used herein, a “subject plant or plant cell” is one in which genetic alteration, such as a mutation, has been effected as to a gene of interest, or is a plant or plant cell which is descended from a plant or cell so altered and which comprises the alteration. As used herein, the term “mutated” or “genetically modified” or “transgenic” or “transformed” or “edited” plants, plant cells, plant tissues, plant parts or seeds refers plants, plant cells, plant tissues, plant parts or seeds that have been mutated by the methods of the present disclosure to include one or more mutations (e.g., insertions, substitutions, or deletions) in the genomic sequence.

As used herein, a “control plant” or “control plant part” or “control cell” or “control seed” refers to a plant or plant part or plant cell or seed that has not been subject to the methods and compositions described herein. A “control” or “control plant” or “control plant part” or “control cell” or “control seed” provides a reference point for measuring changes in phenotype of the subject plant or plant cell. A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e. with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli (e.g., sucrose) that would induce expression of the gene of interest; or (e) the subject plant or plant cell itself, under conditions in which the gene of interest is not expressed. In certain instances, a control plant of the present disclosure is grown under the same environmental conditions (e.g., same or similar temperature, humidity, air quality, soil quality, water quality, and/or pH conditions) as a subject plant described herein. Similarly, a control protein or control protein composition can refer to a protein or protein composition that is isolated or derived from a control plant. In specific embodiments, a control plant, plant part, or plant cell is a plant cell that does not have a mutated nucleotide sequence encoding a SIRT element.

Plant cells possess nuclear, plastid, and mitochondrial genomes. The compositions and methods of the present invention may be used to modify the sequence of the nuclear, plastid, and/or mitochondrial genome, or may be used to modulate the expression of a gene or genes encoded by the nuclear, plastid, and/or mitochondrial genome. Accordingly, by “chromosome” or “chromosomal” is intended the nuclear, plastid, or mitochondrial genomic DNA. “Genome” as it applies to plant cells encompasses not only chromosomal DNA found within the nucleus, but organelle DNA found within subcellular components (e.g., mitochondria or plastids) of the cell.

As used herein, the term “gene” or “coding sequence”, herein used interchangeably, refers to a functional nucleic acid unit encoding a protein, polypeptide, or peptide. As will be understood by those in the art, this functional term includes genomic sequences, cDNA sequences, and smaller engineered gene segments that express, or may be adapted to express proteins, polypeptides, domains, peptides, fusion proteins, and mutants.

As used herein, the term a “nucleic acid”, used interchangeably with a “nucleotide”, refers to a molecule consisting of a nucleoside and a phosphate that serves as a component of DNA or RNA. For instance, nucleic acids include adenine, guanine, cytosine, uracil, and thymine.

As used herein, a “mutation” is any change in a nucleic acid sequence. Nonlimiting examples comprise insertions, deletions, duplications, substitutions, inversions, and translocations of any nucleic acid sequence, regardless of how the mutation is brought about and regardless of how or whether the mutation alters the functions or interactions of the nucleic acid. For example and without limitation, a mutation may produce altered enzymatic activity of a ribozyme, altered base pairing between nucleic acids (e.g. RNA interference interactions, DNA-RNA binding, etc.), altered mRNA folding stability, and/or how a nucleic acid interacts with polypeptides (e.g. DNA-transcription factor interactions, RNA-ribosome interactions, gRNA-endonuclease reactions, etc.). A mutation might result in the production of proteins with altered amino acid sequences (e.g. missense mutations, nonsense mutations, frameshift mutations, etc.) and/or the production of proteins with the same amino acid sequence (e.g. silent mutations). Certain synonymous mutations may create no observed change in the plant while others that encode for an identical protein sequence nevertheless result in an altered plant phenotype (e.g. due to codon usage bias, altered secondary protein structures, etc.). Mutations may occur within coding regions (e.g., open reading frames) or outside of coding regions (e.g., within promoters, terminators, untranslated elements, or enhancers), and may affect, for example and without limitation, gene expression levels, gene expression profiles, protein sequences, and/or sequences encoding RNA elements such as tRNAs, ribozymes, ribosome components, and microRNAs.

Accordingly, “plant with mutation” or “plant part with mutation” or “plant cell with mutation” or “plant genome with mutation” refers to a plant or plant part or plant cell or plant genome that contains a mutation (e.g., an insertion, a substitution, or a deletion) described in the present disclosure, such as a mutated nucleotide sequence encoding a SIRT element and/or a mutated SIRT element (e.g., SlbZIP1 SIRT). For example, as used herein, a plant, plant part or plant cell with mutation may refer to a plant, plant part or plant cell in which, or in an ancestor of which, a SIRT element has been deliberately mutated such that the plant, plant part or plant cell expresses a mutated (e.g., truncated) SIRT peptide. The mutated SIRT peptide can have altered function, e.g., reduced function or loss-of-function, compared to a wild-type, or control, SIRT peptide comprising no mutation.

“Genome editing” or “gene editing” as used herein refers to a type of genetic engineering by which one or more mutations (e.g., insertions, substitutions, deletions, modifications) are introduced at a specific location of the genome. A “gene editing system”, as used herein, refers to a set of molecules or a construct comprising or encoding the molecules for introducing one or more mutations in the genome. An exemplary gene editing system comprises a nuclease and a guide RNA. For example, a CRISPR system comprises a CRISPR nuclease (e.g., CRISPR (clustered regularly interspaced short palindromic repeats)-associated (Cas) endonuclease or a variant thereof, such as Cas12a) and a guide RNA. A CRISPR nuclease associates with a guide RNA that directs nucleic acid cleavage by the associated endonuclease by hybridizing to a recognition site in a polynucleotide. The guide RNA comprises a direct repeat and a guide sequence, which is complementary to the target recognition site. In certain embodiments, the CRISPR system further comprises a tracrRNA (trans-activating CRISPR RNA) that is complementary (fully or partially) to the direct repeat sequence present on the guide RNA. A “TALEN” nuclease is an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, and yeast HO endonuclease. A “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, and yeast HO endonuclease.

As used herein, the terms “nuclease” and “endonuclease” are used interchangeably to refer to naturally-occurring or engineered enzymes, which cleave a phosphodiester bond within a polynucleotide chain.

As used herein, the term “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

An expression construct can permit transcription of a particular polynucleic acid sequence in a host cell (e.g., a bacterial cell or a plant cell). An expression cassette may be part of a plasmid, viral genome, or nucleic acid fragment. Typically, an expression cassette includes a polynucleotide to be transcribed, operably linked to a promoter. “Operably linked” is intended to mean a functional linkage between two or more elements. For example, an operable linkage between a promoter of the present invention and a heterologous nucleotide is a functional link that allows for expression of the heterologous nucleic acid molecule. Operably linked elements may be contiguous or non-contiguous. When used to refer to the joining of two protein coding regions, by operably linked is intended that the coding regions are in the same reading frame. The cassette may additionally contain at least one additional gene to be co-transformed into the plant. Alternatively, the additional gene(s) can be provided on multiple expression cassettes or DNA constructs. The expression cassette may additionally contain selectable marker genes. Other elements that may be present in an expression cassette include those that enhance transcription (e.g., enhancers) and terminate transcription (e.g., terminators), as well as those that confer certain binding affinity or antigenicity to the recombinant protein produced from the expression cassette.

As used herein, “function” of a gene, a peptide, a protein, or a molecule refers to activity of a gene, a peptide, a protein, or a molecule. For example, the term “SIRT activity” or “SIRT function” refers to the ability of the SIRT element to regulate a downstream ORF (e.g., bZIP), such as to repress a downstream ORF (e.g., bZIP) in the presence of sucrose. In specific embodiments a downstream ORF is in-frame with the SIRT element. The downstream ORF can be immediately downstream of the SIRT element coding sequence or can have intervening sequences, such as an intervening gene or regulatory region between the SIRT coding sequence and the downstream ORF. In some instances, “SIRT activity” or “SIRT function” may also refer to the ability of the SIRT element to regulate expression or function of molecules downstream of bZIP, e.g., asparagine synthase (ASN), proline dehydrogenase (PDH), sucrose phosphate synthase (SPS), sucrose-6′-phosphate phosphatase (SPP), or calmodulin. Accordingly, “reduced function” or “loss of function” of the SIRT element may refer to reduced ability or loss of ability of the SIRT element to regulate a downstream ORF (e.g., bZIP), such as to repress a downstream ORF (e.g., bZIP) in the presence of sucrose; or reduced ability or loss of ability of the SIRT element to regulate expression or function of molecules downstream of bZIP, e.g., asparagine synthase (ASN), proline dehydrogenase (PDH), sucrose phosphate synthase (SPS), sucrose-6′-phosphate phosphatase (SPP), or calmodulin. A mutated upstream regulatory region of bZIP (e.g., bZIP SIRT) in a plant or plant part, or introducing mutations to the upstream regulatory region of bZIP (e.g., bZIP SIRT) in a plant or plant part may cause reduced function or loss of function of the SIRT element.

“Introduced” in the context of inserting a nucleic acid molecule (e.g., a recombinant DNA construct) into a cell, means “transfection” or “transformation” or “transduction” and includes reference to the incorporation of a nucleic acid fragment into a plant cell where the nucleic acid fragment may be incorporated into the genome of the cell (e.g., nuclear chromosome, plasmid, plastid chromosome or mitochondrial chromosome), converted into an autonomous replicon, or transiently expressed (e.g., transfected mRNA).

As used herein with respect to a parameter, the term “decreased” or “decreasing” or “decrease” or “reduced” or “reducing” or “reduce” or “lower” refers to a detectable (e.g., at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%) negative change in the parameter from a comparison control, e.g., an established normal or reference level of the parameter, or an established standard control. Accordingly, the terms “decreased”, “reduced”, and the like encompass both a partial reduction and a complete reduction compared to a control.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

As used herein, the term “polypeptide” refers to a linear organic polymer containing a large number of amino-acid residues bonded together by peptide bonds in a chain, forming part of (or the whole of) a protein molecule. The amino acid sequence of the polypeptide refers to the linear consecutive arrangement of the amino acids comprising the polypeptide, or a portion thereof.

As used herein the term “polynucleotide” refers to a single or double stranded nucleic acid sequence which is isolated and provided in the form of an RNA sequence (e.g., an mRNA sequence), a complementary polynucleic acid sequence (cDNA), a genomic polynucleic acid sequence and/or a composite polynucleic acid sequences (e.g., a combination of the above).

The term “isolated” refers to at least partially separated from the natural environment e.g., from a plant cell.

As used herein, the term “expression” or “expressing” refers to the transcription and/or translation of a particular nucleic acid sequence driven by a promoter.

As used herein, the terms “exogenous” or “heterologous” in reference to a nucleic acid sequence or amino acid sequence are intended to mean a sequence that is purely synthetic, that originates from a foreign species, or, if from the same species, is substantially modified from its native form in composition and/or genomic locus by deliberate human intervention. Thus, a heterologous nucleic acid sequence may not be naturally expressed within the plant (e.g., a nucleic acid sequence from a different species) or may have altered expression when compared to the corresponding wild type plant. An exogenous polynucleotide may be introduced into the plant in a stable or transient manner, so as to produce a ribonucleic acid (RNA) molecule and/or a polypeptide molecule. It should be noted that the exogenous polynucleotide may comprise a nucleic acid sequence which is identical or partially homologous to an endogenous nucleic acid sequence of the plant.

As used herein, the term “endogenous” in reference to a gene or nucleic acid sequence or protein is intended to mean a gene or nucleic acid sequence or protein that is naturally comprised within or expressed by a cell. Endogenous genes can include genes that naturally occur in the cell of a plant, but that have been modified in the genome of the cell without insertion or replacement of a heterologous gene that is from another plant species or another location within the genome of the modified cell.

As used herein, “fertilization” and/or “crossing” broadly includes bringing the genomes of gametes together to form zygotes but also broadly may include pollination, syngamy, fecundation and other processes related to sexual reproduction. Typically, a cross and/or fertilization occurs after pollen is transferred from one flower to another, but those of ordinary skill in the art will understand that plant breeders can leverage their understanding of fertilization and the overlapping steps of crossing, pollination, syngamy, and fecundation to circumvent certain steps of the plant life cycle and yet achieve equivalent outcomes, for example, a plant or cell of a soybean cultivar described herein. In certain embodiments, a user of this innovation can generate a plant of the claimed invention by removing a genome from its host gamete cell before syngamy and inserting it into the nucleus of another cell. While this variation avoids the unnecessary steps of pollination and syngamy and produces a cell that may not satisfy certain definitions of a zygote, the process falls within the definition of fertilization and/or crossing as used herein when performed in conjunction with these teachings. In certain embodiments, the gametes are not different cell types (i.e. egg vs. sperm), but rather the same type and techniques are used to effect the combination of their genomes into a regenerable cell. Other embodiments of fertilization and/or crossing include circumstances where the gametes originate from the same parent plant, i.e. a “self” or “self-fertilization”. While selfing a plant does not require the transfer pollen from one plant to another, those of skill in the art will recognize that it nevertheless serves as an example of a cross, just as it serves as a type of fertilization. Thus, methods and compositions taught herein are not limited to certain techniques or steps that must be performed to create a plant or an offspring plant of the claimed invention, but rather include broadly any method that is substantially the same and/or results in compositions of the claimed invention.

“Homolog” or “homologous sequence” may refer to both orthologous and paralogous sequences. Paralogous sequence relates to gene-duplications within the genome of a species. Orthologous sequence relates to homologous genes in different organisms due to ancestral relationship. Thus, orthologs are evolutionary counterparts derived from a single ancestral gene in the last common ancestor of given two species and therefore have great likelihood of having the same function. One option to identify homologs (e.g., orthologs) in monocot plant species is by performing a reciprocal BLAST search. This may be done by a first blast involving blasting the sequence-of-interest against any sequence database, such as the publicly available NCBI database which may be found at: ncbi.nlm.nih.gov. If orthologs in rice were sought, the sequence-of-interest would be blasted against, for example, the 28,469 full-length cDNA clones from Oryza sativa Nipponbare available at NCBI. The blast results may be filtered. The full-length sequences of either the filtered results or the non-filtered results are then blasted back (second blast) against the sequences of the organism from which the sequence-of-interest is derived. The results of the first and second blasts are then compared. An ortholog is identified when the sequence resulting in the highest score (best hit) in the first blast identifies in the second blast the query sequence (the original sequence-of-interest) as the best hit. Using the same rational a paralog (homolog to a gene in the same organism) is found. In case of large sequence families, the ClustalW program may be used [ebi.ac.uk/Tools/clustalw2/index.html], followed by a neighbor-joining tree (wikipedia.org/wiki/Neighbor-joining) which helps visualizing the clustering.

In some embodiments, the term “homolog” as used herein, refers to functional homologs of genes. A functional homolog is a gene encoding a polypeptide that has sequence similarity to a polypeptide encoded by a reference gene, and the polypeptide encoded by the homolog carries out one or more of the biochemical or physiological function(s) of the polypeptide encoded by the reference gene. In general, it is preferred that functional homologs and/or polypeptides encoded by functional homologs share at least some degree of sequence identity with the reference gene or polypeptide encoded by the reference gene.

Homology (e.g., percent homology, sequence identity+sequence similarity) can be determined using any homology comparison software computing a pairwise sequence alignment.

As used herein, “sequence identity,” “identity,” “percent identity,” “percentage similarity,” “sequence similarity” and the like refer to a measure of the degree of similarity of two sequences based upon an alignment of the sequences that maximizes similarity between aligned amino acid residues or nucleotides, and which is a function of the number of identical or similar residues or nucleotides, the number of total residues or nucleotides, and the presence and length of gaps in the sequence alignment. A variety of algorithms and computer programs are available for determining sequence similarity using standard parameters. As used herein, sequence similarity is measured using the BLASTp program for amino acid sequences and the BLASTn program for nucleic acid sequences, both of which are available through the National Center for Biotechnology Information (www.ncbi.nlm.nih.gov/), and are described in, for example, Altschul et al. (1990), J. Mol. Biol. 215:403-410; Gish and States (1993), Nature Genet. 3:266-272; Madden et al. (1996), Meth. Enzymol. 266:131-141; Altschul et al. (1997), Nucleic Acids Res. 25:3389-3402); Zhang et al. (2000), J. Comput. Biol. 7(1-2):203-14. As used herein, percent similarity of two amino acid sequences is the score based upon the following parameters for the BLASTp algorithm: word size=3; gap opening penalty=−11; gap extension penalty=−1; and scoring matrix=BLOSUM62. As used herein, percent similarity of two nucleic acid sequences is the score based upon the following parameters for the BLASTn algorithm: word size=11; gap opening penalty=−5; gap extension penalty=−2; match reward=1; and mismatch penalty=−3. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G. (Proc Natl Acad Sci 89:10915-9 (1992)). Identity (e.g., percent homology) can be determined using any homology comparison software, including for example, the BlastN software of the National Center of Biotechnology Information (NCBI) such as by using default parameters.

According to some embodiments, the identity is a global identity, i.e., an identity over the entire amino acid or nucleic acid sequences of the invention and not over portions thereof.

According to some embodiments, the term “homology” or “homologous” refers to identity of two or more nucleic acid sequences; or identity of two or more amino acid sequences; or the identity of an amino acid sequence to one or more nucleic acid sequence. According to some embodiments, the homology is a global homology, e.g., a homology over the entire amino acid or nucleic acid sequences of the invention and not over portions thereof. The degree of homology or identity between two or more sequences can be determined using various known sequence comparison tools which are described in WO2014/102774.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “population” refers to a set comprising any number, including one, of individuals, objects, or data from which samples are taken for evaluation, e.g., estimating quantitative trait locus (QTL) effects and/or disease tolerance. Most commonly, the terms relate to a breeding population of plants from which members are selected and crossed to produce progeny in a breeding program. A population of plants can include the progeny of a single breeding cross or a plurality of breeding crosses and can be either actual plants or plant derived material, or in silico representations of plants. The member of a population need not be identical to the population members selected for use in subsequent cycles of analyses, nor does it need to be identical to those population members ultimately selected to obtain a final progeny of plants. Often, a plant population is derived from a single biparental cross but can also derive from two or more crosses between the same or different parents. Although a population of plants can comprise any number of individuals, those of skill in the art will recognize that plant breeders commonly use population sizes ranging from one or two hundred individuals to several thousand, and that the highest performing 5-20% of a population is what is commonly selected to be used in subsequent crosses in order to improve the performance of subsequent generations of the population in a plant breeding program.

As used herein, the term “crop performance” is used synonymously with “plant performance” and refers to of how well a plant grows under a set of environmental conditions and cultivation practices. Crop performance can be measured by any metric a user associates with a crop's productivity (e.g., yield), appearance and/or robustness (e.g., color, morphology, height, biomass, maturation rate, etc.), product quality (e.g., fiber lint percent, fiber quality, seed protein content, seed carbohydrate content, etc.), cost of goods sold (e.g., the cost of creating a seed, plant, or plant product in a commercial, research, or industrial setting) and/or a plant's tolerance to disease (e.g., a response associated with deliberate or spontaneous infection by a pathogen) and/or environmental stress (e.g., drought, flooding, low nitrogen or other soil nutrients, wind, hail, temperature, day length, etc.). Crop performance can also be measured by determining a crop's commercial value and/or by determining the likelihood that a particular inbred, hybrid, or variety will become a commercial product, and/or by determining the likelihood that the offspring of an inbred, hybrid, or variety will become a commercial product. Crop performance can be a quantity (e.g., the volume or weight of seed or other plant product measured in liters or grams) or some other metric assigned to some aspect of a plant that can be represented on a scale (e.g., assigning a 1-10 value to a plant based on its disease tolerance).

A “microbe” will be understood to be a microorganism, i.e. a microscopic organism, which can be single celled or multicellular. Microorganisms are very diverse and include all the bacteria, archaea, protozoa, fungi, and algae, especially cells of plant pathogens and/or plant symbionts. Certain animals are also considered microbes, e.g. rotifers. In various embodiments, a microbe can be any of several different microscopic stages of a plant or animal. Microbes also include viruses, viroids, and prions, especially those which are pathogens or symbionts to crop plants. A “pathogen” as used herein refers to a microbe that causes disease or harmful effects on plant health.

A “fungus” includes any cell or tissue derived from a fungus, for example whole fungus, fungus components, organs, spores, hyphae, mycelium, and/or progeny of the same. A fungus cell is a biological cell of a fungus, taken from a fungus or derived through culture of a cell taken from a fungus.

A “pest” is any organism that can affect the performance of a plant in an undesirable way. Common pests include microbes, animals (e.g. insects and other herbivores), and/or plants (e.g. weeds). Thus, a pesticide is any substance that reduces the survivability and/or reproduction of a pest, e.g. fungicides, bactericides, insecticides, herbicides, and other toxins.

“Tolerance” or “improved tolerance” in a plant to disease conditions (e.g. growing in the presence of a pest) will be understood to mean an indication that the plant is less affected by the presence of pests and/or disease conditions with respect to yield, survivability and/or other relevant agronomic measures, compared to a less tolerant, more “susceptible” plant. Tolerance is a relative term, indicating that a “tolerant” plant survives and/or performs better in the presence of pests and/or disease conditions compared to other (less tolerant) plants (e.g., a different soybean cultivar) grown in similar circumstances. As used in the art, “tolerance” is sometimes used interchangeably with “resistance”, although resistance is sometimes used to indicate that a plant appears maximally tolerant to, or unaffected by, the presence of disease conditions. Plant breeders of ordinary skill in the art will appreciate that plant tolerance levels vary widely, often representing a spectrum of more-tolerant or less-tolerant phenotypes, and are thus trained to determine the relative tolerance of different plants, plant lines or plant families and recognize the phenotypic gradations of tolerance.

“Yield” as used herein is defined as the measurable produce of economic value from a crop. This may be defined in terms of quantity and/or quality. Yield is directly dependent on several factors, for example, the number and size of the organs, plant architecture (for example, the number of branches), seed production, leaf senescence and more. Root development, nutrient uptake, stress tolerance, photosynthetic carbon assimilation rates, and early vigor may also be important factors in determining yield. Optimizing the abovementioned factors may therefore contribute to increasing crop yield. Yield can be measured and expressed by any means known in the art. In specific embodiments, yield is measured by seed weight or volume in a given harvest area.

A plant, or its environment, can be contacted with a wide variety of “agriculture treatment agents.” As used herein, an “agriculture treatment agent”, or “treatment agent”, or “agent” can refer to any exogenously provided compound that can be brought into contact with a plant tissue (e.g. a seed) or its environment that affects a plant's growth, development and/or performance, including agents that affect other organisms in the plant's environment when those effects subsequently alter a plant's performance, growth, and/or development (e.g. an insecticide that kills plant pathogens in the plant's environment, thereby improving the ability of the plant to tolerate the insect's presence). Agriculture treatment agents also include a broad range of chemicals and/or biological substances that are applied to seeds, in which case they are commonly referred to as seed treatments and/or seed dressings. Seed treatments are commonly applied as either a dry formulation or a wet slurry or liquid formulation prior to planting and, as used herein, generally include any agriculture treatment agent including growth regulators, micronutrients, nitrogen-fixing microbes, and/or inoculants. Agriculture treatment agents include pesticides (e.g. fungicides, insecticides, bactericides, etc.) hormones (abscisic acids, auxins, cytokinins, gibberellins, etc.) herbicides (e.g. glyphosate, atrazine, 2,4-D, dicamba, etc.), nutrients (e.g. a plant fertilizer), and/or a broad range of biological agents, for example a seed treatment inoculant comprising a microbe that improves crop performance, e.g. by promoting germination and/or root development. In certain embodiments, the agriculture treatment agent acts extracellularly within the plant tissue, such as interacting with receptors on the outer cell surface. In some embodiments, the agriculture treatment agent enters cells within the plant tissue. In certain embodiments, the agriculture treatment agent remains on the surface of the plant and/or the soil near the plant. In certain embodiments, the agriculture treatment agent is contained within a liquid. Such liquids include, but are not limited to, solutions, suspensions, emulsions, and colloidal dispersions. In some embodiments, liquids described herein will be of an aqueous nature. However, in various embodiments, such aqueous liquids that comprise water can also comprise water insoluble components, can comprise an insoluble component that is made soluble in water by addition of a surfactant, or can comprise any combination of soluble components and surfactants. In certain embodiments, the application of the agriculture treatment agent is controlled by encapsulating the agent within a coating, or capsule (e.g. microencapsulation). In certain embodiments, the agriculture treatment agent comprises a nanoparticle and/or the application of the agriculture treatment agent comprises the use of nanotechnology. In some embodiments, the plants described herein can grow in the presence of one or more agricultural treatment agents. For example, the plants described herein can have an increased sugar content and can grow in the presence of commonly used herbicides.

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference.

All publications, patent applications, patents, and other references mentioned herein are incorporated by reference herein in their entirety.

### II. Overview of the Invention

Consumers prefer sweet fruits and vegetables. Thus, increasing the sugar content of a fruit or vegetable will improve the taste profile. The metric for a desirable trait includes an increase in total soluble solids (TSS) and/or sugar levels of the mature fruit without negatively impacting plant agronomics and/or acid/volatile levels. Modification of carbohydrate metabolism or carbohydrate transport in plants or plant parts is a strategy to produce plant or plant parts with increased sugar and/or TSS levels. Sucrose serves a vital role as a signaling molecule to regulate gene expression and pathways involved in carbohydrate metabolism or transport in plants. For example, basic region leucine zipper (bZIP) transcription factor genes have a conserved sucrose-sensitive sequence upstream open reading frame (ORF), termed “sucrose-induced repression of translation (SIRT)”, that represses the downstream main ORF. The bZIP transcription factor gene is normally under regulation by the upstream SIRT element through feedback inhibition by sucrose accumulation.

In tomato plants (Solanum lycopersicum), two bZIP family transcription factor homologs were identified (SlbZIP1 and SlbZIP2). SlbZIP1 (also known as bZIP transcription factor 6, LeZIP2, and bZIP1) and SlbZIP2 are orthologs of tobacco tbzF and tbz17, respectively. Exemplary nucleotide sequences and amino acid sequences of SlbZIP1 and SlbZIP2 can be found, for example, at GenBank Accession No. NM_032790.3 and XM_004240524.4, respectively. SlbZIP1 and SlbZIP2 both contain an upstream SIRT element. The terms “basic leucine zipper transcription factor” and “bZIP,” as used herein, also refer to naturally occurring DNA sequence variations of the bZIP transcription factor gene. The entire contents of each of the foregoing GenBank Accession numbers and the Gene database numbers are incorporated herein by reference as of the date of filing this application. Both SlbZIP1 and SlbZIP2 are expressed in tomato leaf and fruit tissue. In specific embodiments, a bZIP1 transcription factor refers to the SlbZIP1 transcription factor encoded by the nucleic acid sequence set forth in SEQ ID NO: 21, or a fragment or variant thereof that encodes a functional bZIP1 transcription factor. Variants of the SlbZIP1 transcription factor retain bZIP1 activity and are encoded by a nucleic acid molecule with a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleic acid sequence set forth in SEQ ID NO: 21. In specific embodiments, a bZIP2 transcription factor refers to the SlbZIP2 transcription factor encoded by the nucleic acid sequence set forth in SEQ ID NO: 22, or a fragment or variant thereof that encodes a functional bZIP1 transcription factor. Variants of the SlbZIP2 transcription factor retain bZIP2 activity and are encoded by a nucleic acid molecule with a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleic acid sequence set forth in SEQ ID NO: 22.

Overexpression of SlbZIP1 and SlbZIP2 genes enhanced expression of asparagine synthetase (SlASN1) and proline dehydrogenase (SlProDH1, SlProDH2). Further downstream metabolic remodeling resulted in increases in expression of sucrose phosphate synthase (SlSPS1, SlSPS2), sucrose phosphatase (SlSPP), and calmodulin (SlCaM2), resulting in modulation of sugar metabolism and sugar levels in tomatoes. Transforming tomato plants with vectors encoding the tomato SlbZIP1 gene without the functional SIRT element in the upstream ORF resulted in increased sugar levels, altered amino acid compositions, and increased total amino acid accumulation in transgenic tomato fruits compared to wild-type plants. Accordingly, mutations in a bZIP transcription factor gene or a homolog thereof could alter (e.g., increase or decrease) sugar levels and/or total soluble solids levels in fruits and vegetables.

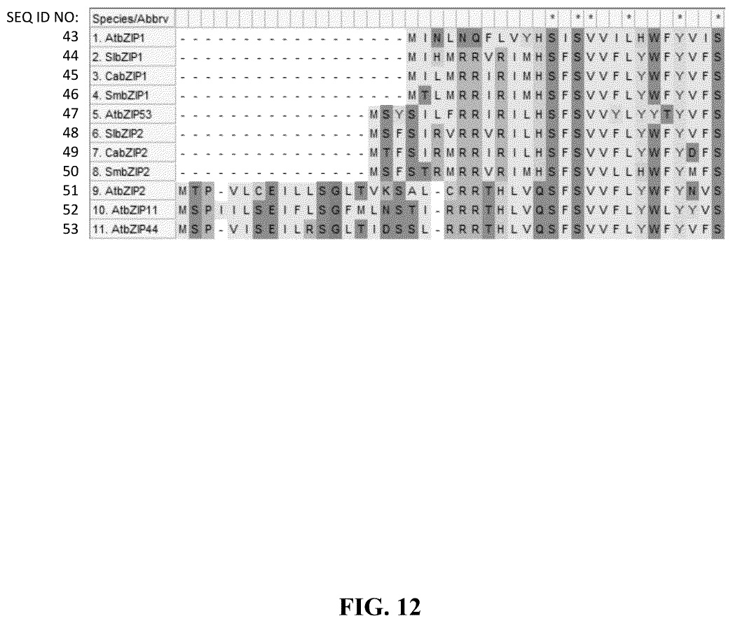

As shown in FIG. 12, the orthologs of bZIP1 and bZIP2 in other common vegetable crops such as pepper (Capsicum annuum) and eggplant (Solanum melongena) also contain SIRT elements which are highly conserved toward the C-terminus of the peptide sequence. Disrupting the SIRT elements of these vegetable crops could also modulate the expression of carbohydrate metabolism genes and increase the sugar content of these commercially relevant crops, adding value to their acceptance by consumers. A novel single gene trait that increases sugar content could offer new fruit or vegetable varieties with improved flavor and consumer acceptance.

### III. Plants with Increased Sugar Content

Disclosed herein are plants or plant parts comprising altered function or activity of a basic region/leucine zipper motif (bZIP) transcription factor gene or a homolog thereof. The plants or plant parts have one or more insertions, substitutions, or deletions in a 5′ untranslated region (5′ UTR) of a bZIP transcription factor gene or a homolog thereof, wherein the 5′ UTR comprises an upstream open reading frame (uORF) with a sucrose induced repression of translation (SIRT) element. The one or more insertions, substitutions, or deletions alter function of the bZIP SIRT element. The wild-type SIRT element can reduce the expression of a bZIP transcription factor in the presence of sucrose. The plants or plant parts can, therefore, have increased sugar content relative to a control plant or plant part not comprising the one or more insertions, substitutions, or deletions in the uORF of the bZIP transcription factor gene or a homolog thereof.

The teachings herein are not limited to certain plant species, and it is envisioned that they can be modified to be useful for monocots, dicots, and/or substantially any crop and/or valuable plant type, including plants that can reproduce by self-fertilization and/or cross fertilization, hybrids, inbreds, varieties, and/or cultivars thereof. A plant or plant part of the present disclosure can be a crop plant or part of a crop plant. Examples of crop plants include, but are not limited to, tomato (Solanum lycopersicum), eggplant (Solanum melongena), pepper (Capsicum annuum), corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana), sunflower (Helianthus annuus), quinoa (Chenopodium quinoa), chicory (Cichorium intybus), lettuce (Lactuca sativa), safflower (Carthamus tinctorius), wheat (Triticum aestivum), soybean (Glycine max), tobacco (Nicotiana spp., e.g., Nicotiana tabacum, Nicotiana sylvestris), potato (Solanum tuberosum), tomato (Solanum lycopersicum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), grapes (Vitis vinifera, Vitis riparia), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), pea (Pisum sativum), eucalyptus (Eucalyptus spp.), oats (Avena sativa), barley (Hordeum vulgare), vegetables, ornamentals, and conifers. Additionally, a plant or plant part of the present disclosure can be an oilseed plant (e.g., canola (Brassica napus), cotton (Gossypium sp.), camelina (Camelina sativa) and sunflower (Helianthus sp.)), or other species including wheat (Triticum sp., such as Triticum aestivum L. ssp. aestivum (common or bread wheat), other subspecies of Triticum aestivum, Triticum turgidum L. ssp. durum (durum wheat, also known as macaroni or hard wheat), Triticum monococcum L. ssp. monococcum (cultivated einkom or small spelt), Triticum timopheevi ssp. timopheevi, Triticum turgigum L. ssp. dicoccon (cultivated emmer), and other subspecies of Triticum turgidum (Feldman)), barley (Hordeum vulgare), maize (Zea mays), oats (Avena sativa), or hemp (Cannabis sativa). Additionally, a plant or plant part of the present disclosure can be a forage plant or part of a forage plant. Examples of forage plants include legumes and crop plants described herein as well as grass forages including Agrostis spp., Lolium spp., Festuca spp., Poa spp., and Bromus spp. A plant or plant part of the present disclosure can be a legume, i.e., a plant belonging to the family Fabaceae (or Leguminosae), or a part (e.g., fruit or seed) of such a plant. Examples of legume include, without limitation, soybean (Glycine max), beans (Phaseolus spp., Vigna spp.), common bean (Phaseolus vulgaris), mung bean (Vigna radiata), cowpea (Vigna unguiculata), adzuki bean (Vigna angularis), fava bean (Vicia faba), pea (Pisum sativum), chickpea (Cicer arietinum), peanut (Arachis hypogaea), lentils (Lens culinaris, Lens esculenta), lupins (Lupinus spp.), white lupin (Lupinus albus), mesquite (Prosopis spp.), carob (Ceratonia siliqua), tamarind (Tamarindus indica), alfalfa (Medicago sativa), barrel medic (Medicago truncatula), birdsfood trefoil (Lotus japonicus), licorice (Glycyrrhiza glabra), and clover (Trifolium spp.). For example, a plant or plant part of the present disclosure can be Glycine max or Pisum sativum.

A. Plants with Mutation in SIRT Sequence

In some aspects, the present disclosure provides plants or plant parts thereof, including fruits and seeds, with increased sugar content, comprising one or more mutations (e.g., insertions, substitutions, or deletions) in a 5′ UTR of a bZIP transcription factor gene or a homolog thereof, wherein the 5′ UTR comprises an uORF, and wherein the uORF comprises a SIRT element. In some aspects, the plants or plant parts have one or more mutations in the uORF of bZIP coding sequence. For example, the plants or plant parts have one or more mutations in the SIRT element of the uORF of a bZIP coding sequence. In particular, described herein are plants or plant parts, in which an upstream regulatory region of the bZIP transcription factor gene (e.g., the bZIP SIRT element) has been mutated, e.g., by one or more (e.g., about 1, 2, 3, 4, 5, 6, 7, 8, 9, 10, 11, 12, 13, 14, 15, 16, 17, 18, 19, 20, or more) insertions, substitutions or deletions in the genome of cells or parts of the plants. For example, disclosed herein are plants or plant parts in which a gene encoding the SIRT peptide has been mutated, e.g., by one or more insertions, substitutions or deletions in the SIRT coding sequence. In some embodiments, the one or more mutations (e.g., insertions, substitutions, or deletions) comprise an out-of-frame mutation of the SIRT element. In some embodiments, the one or more insertions, substitutions, or deletions comprise an in-frame mutation of the SIRT element.

In specific embodiments, a control plant or plant part described herein (e.g., a plant or plant part to which mutations can be introduced by the methods of the present disclosure) can have a SIRT peptide (e.g., SlbZIP1 SIRT) that comprises a wild-type amino acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence set forth in SEQ ID NO: 5 or 6. For example, a plant or plant part described herein can have a SIRT peptide that comprises the amino acid sequence of SEQ ID NO: 5 or 6.

A control plant or plant part described herein (e.g., a plant or plant part to which mutations can be introduced by the methods of the present disclosure) can comprise a SIRT (e.g., SlbZIP1 SIRT) nucleic acid sequence that encodes a SIRT peptide. A SIRT nucleic acid sequence can comprise at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence set forth in SEQ ID NO: 3 or 4. For example, a control plant or plant part described herein can have a SIRT gene that comprises the nucleic acid sequence of SEQ ID NO: 3 or 4. A plant or plant part described herein can comprise 1-6, 2-4, 3-4, 2-5, or 3-5 (e.g., 1, 2, 3, 4, 5, or 6) copies of SIRT element (e.g., SlbZIP1 SIRT). In particular, a plant or plant part described herein can comprise at least 2 genes encoding a SIRT peptide, such as 2 genes that have less than 100% (e.g., less than 100%, 99%, 98%, 97%, 96%, 95%, 94%, 93%, 92%, 91%, 90%, 89%, 88%, 87%, 86%, or 85%) sequence identity to each other.

A control plant or plant part described herein (e.g., a plant or plant part to which mutations can be introduced by, e.g., the methods of the present disclosure) may comprise a SIRT element (e.g., SlbZIP1 SIRT) that retains SIRT function. Without wishing to be bound by theory, the SIRT element represses translation of bZIP in the presence of sucrose, which in turn regulates molecules downstream of bZIP that regulate carbohydrate metabolism and/or transport in plants. Thus, a plant or plant part described herein may comprises a SIRT element (e.g., SlbZIP1 SIRT) with an altered (e.g., reduced, loss of) ability to regulate the downstream bZIP transcription factor and genes downstream of bZIP regulated by bZIP.

Also disclosed are variants and fragments of sequences (e.g., bZIP, bZIP SIRT) of the present disclosure. Such sequences include sequences that are orthologs of the disclosed sequences. “Orthologs” is intended to mean genes derived from a common ancestral gene and which are found in different species as a result of speciation. Genes found in different species are considered orthologs when their nucleic acid sequences and/or their encoded protein sequences share at least 75%, 80%, 85%, 90%, 91%, 92%, 93%, 94%, 95%, 96%, 97%, 98%, 99%, or greater sequence identity. Functions of orthologs are often highly conserved among species. Thus, isolated polynucleotides that have transcription activation or enhancer activities and which share at least 75% sequence identity to the sequences disclosed herein, or to variants or fragments thereof, are encompassed by the present disclosure. For example, orthologs of bZIP include, but not limited Capsicum annuum bZIP1, Capsicum annuum bZIP2, Solanum melongena bZIP1, and Solanum melongena bZIP2. Nucleic acid sequence encoding Capsicum annuum bZIP1, Capsicum annuum bZIP2, Solanum melongena bZIP1, and Solanum melongena bZIP2 are set forth as SEQ ID NOs: 27-30, respectively. Amino acid sequence of Capsicum annuum bZIP1, Capsicum annuum bZIP2, Solanum melongena bZIP1, and Solanum melongena bZIP2 are set forth as SEQ ID NOs: 31-34, respectively. Nucleic acid sequence for SIRT element Capsicum annuum bZIP1, Capsicum annuum bZIP2, Solanum melongena bZIP1, and Solanum melongena bZIP2 are set forth as SEQ ID NOs: 35-38, respectively. Amino acid sequence for SIRT element of Capsicum annuum bZIP1, Capsicum annuum bZIP2, Solanum melongena bZIP1, and Solanum melongena bZIP2 are set forth as SEQ ID NOs: 39-42, respectively. These bZIP orthologs, SIRT elements, and plants and plant parts comprising these bZIP orthologs and/or SIRT elements are encompassed by the present disclosure. Variant sequences can be isolated by PCR. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, New York). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York).

Variant sequences may also be identified by analysis of existing databases of sequenced genomes. In this manner, variant sequences encoding, for instance, bZIP or bZIP SIRT element can be identified and used in the methods of the present disclosure. The variant sequences will retain the biological activity.

In some embodiments, the plants or plant parts of the present disclosure may comprise one or more insertions, substitutions or deletions in a nucleotide region corresponding to the 5′UTR of the bZIP transcription factor gene. For example, the plants or plant parts may comprise one or more insertions, substitutions or deletions of about 1-84, 2-84, 3-84, 4-84, 5-84, 6-84, 7-84, 8-84, 9-84, 10-84, or 11-84 (e.g., about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, or 84) nucleotides of bZIP 5′UTR in the genome of a plant cell or plant part. In particular, the plants or plant parts may comprise one or more insertions, substitutions or deletions of about 7-9 nucleotides in the bZIP 5′UTR.

In some embodiments, the plants or plant parts of the present disclosure may comprise one or more insertions, substitutions or deletions in a nucleotide region corresponding to the uORF of the bZIP transcription factor gene. For example, the plants or plant parts may comprise one or more insertions, substitutions or deletions of about 1-84, 2-84, 3-84, 4-84, 5-84, 6-84, 7-84, 8-84, 9-84, 10-84, or 11-84 (e.g., about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, or 84) nucleotides of bZIP uORF in the genome of a plant cell or plant part. In particular, the plants or plant parts may comprise one or more insertions, substitutions or deletions of about 7-9 nucleotides in the bZIP uORF.

In some embodiments, the plants or plant parts of the present disclosure may comprise one or more insertions, substitutions or deletions in a nucleotide region corresponding to the SIRT element of the bZIP transcription factor gene. For example, the plants or plant parts may comprise one or more insertions, substitutions or deletions of about 1-84, 2-84, 3-84, 4-84, 5-84, 6-84, 7-84, 8-84, 9-84, 10-84, or 11-84 (e.g., about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, or 84) nucleotides of bZIP SIRT element in the genome of a plant cell or plant part. In particular, the plants or plant parts may comprise one or more insertions, substitutions or deletions of about 7-9 nucleotides in the bZIP SIRT element. The mutation (e.g., insertion, substitution, or deletion) may be an in-frame mutation or an out-of-frame mutation.

The one or more insertions, substitutions or deletions in the bZIP upstream regulatory region (e.g., bZIP SIRT element) may be in a nucleotide region that comprises a nucleic acid sequence having at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence of SEQ ID NO: 3 or 4.

For example, the one or more insertions, substitutions or deletions in in the bZIP upstream regulatory region (e.g., bZIP SIRT element) may comprise a deletion of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, or 74 nucleotides in a nucleotide region that comprises the nucleic acid sequence of SEQ ID NO: 3 or 4. In particular, the one or more insertions, substitutions or deletions in the bZIP upstream regulatory region (e.g., bZIP SIRT element) may comprise a deletion of about 7-9 nucleotides. In some embodiments, the deletion is a deletion of nucleotide 6 through 13 of SEQ ID NO: 3, resulting in a sequence set forth in SEQ ID: 7; a deletion of nucleotide 53 through 60 of SEQ ID NO: 3, resulting in the sequence set forth in SEQ ID: 8; a deletion of nucleotide 53 through 61 of SEQ ID NO: 3, resulting in the sequence set forth in SEQ ID: 9; or a deletion of nucleotide 261 through 268 of SEQ ID NO: 1, resulting in a deletion of nucleotide 69 through 75 of SEQ ID NO: 3 and a sequence set forth in SEQ ID: 10. In some embodiments, the mutated SIRT element shares at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) identity with a sequence selected from the group consisting of SEQ ID No: 7-10.

In some embodiments, the one or more insertions, substitutions or deletions in the bZIP upstream regulatory region (e.g., bZIP SIRT element) nucleic acid sequence may produce a peptide comprising one or more amino acid changes (e.g., 1, 2, 3, 4, 5, 6, 7, 8, 9, 10, 11, 12, 13, 14, 15, 16, 17, 18, 19, 20, 21, 22, 23, 24, 25, 26, 27, or 28 amino acid changes) in the amino acid sequence that comprises the amino acid sequence of SEQ ID NO: 5 or 6 in the plant or plant part. In particular, the one or more insertions, substitutions or deletions in the bZIP upstream regulatory region (e.g., bZIP SIRT element) may comprise 1, 2, or 3 amino acid changes in a SIRT peptide sequence. In some embodiments, the change comprises one or more of M1L, M4L, and M10L compared to the amino acid sequence of SEQ ID NO: 5. In some embodiments, the mutated SIRT element shares at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) with the amino acid sequence of SEQ ID NO: 11.

A bZIP 5′UTR, a bZIP uORF, or a bZIP SIRT element of the present disclosure with one or more mutations (e.g., with deletion of about 7-9 nucleotides in the SIRT element, or with changes of about 1, 2, or 3 amino acids in the SIRT peptide sequence) may be found in plants or plant parts to which one or more mutations have been introduced by the methods of the present disclosure. A mutated bZIP SIRT element can also be found in plant parts (e.g., juice, pulp, seed, fruit, flowers, nectar, embryos, pollen, ovules, leaves, stems, branches, bark, kernels, ears, cobs, husks, stalks, roots, root tips, anthers, etc.), plant extract (e.g., sweetener, antioxidants, alkaloids, etc.), plant concentrate (e.g., whole plant concentrate or plant part concentrate), plant powder (e.g., formulated powder, such as formulated plant part powder (e.g., seed flour)), and plant biomass (e.g., dried biomass, such as crushed and/or powdered biomass) obtained from such plants.

In some embodiments, the one or more mutations are integrated into the plant genome and the plant or the plant part is stably transformed. In other embodiments, the one or more mutations are not integrated into the plant genome and wherein the plant or the plant part is transiently transformed.

B. Plants with Altered Function of SIRT and bZIP

Plants or plant parts of the present disclosure, comprising one or more mutations in the upstream regulatory region of the bZIP, or plant products obtained from such plants may have altered function (e.g., loss-of-function or reduced function) in the bZIP SIRT element, as compared to a control plant, plant part, or plant product. “Plant products”, as used herein, refers to any product or composition produced from the plant, including any oil products, sugar products, fiber products, protein products (such as protein concentrate, protein isolate, flake, or other protein product), seed hulls, meal, or flour, for a food, feed, aqua, or industrial product, plant extract (e.g., sweetener, antioxidants, alkaloids, etc.), plant concentrate (e.g., whole plant concentrate or plant part concentrate), plant powder (e.g., formulated powder, such as formulated plant part powder (e.g., seed flour)), plant biomass (e.g., dried biomass, such as crushed and/or powdered biomass), grains, plant protein composition, plant oil composition, and food and beverage products containing plant compositions (e.g., plant parts, plant extract, plant concentrate, plant powder, plant protein, plant oil, and plant biomass) described herein. Plant parts and plant products provided herein can be intended for human or animal consumption.

Plants, plant parts, or plant products of the present disclosure comprising one or more mutations in the upstream regulatory region of the bZIP may have altered function (e.g., loss-of-function or reduced function) in the bZIP transcription factor, as compared to a control plant or plant part. The SIRT element regulates the function of bZIP, including repressing the bZIP function in the presence of sucrose. Accordingly, plants, plant parts, or plant products of the present disclosure may comprise altered expression or function of a molecule downstream of, or regulated by, the bZIP transcription factor compared to a control plant, plant part, or plant product.

A control plant or plant part can be a plant or plant part to which one or more mutations has not be introduced by the methods provided herein. Thus, a control plant or plant part may express wild-type (WT) SIRT element. A control plant of the present disclosure may be grown under the same environmental conditions (e.g., same or similar temperature, humidity, air quality, soil quality, water quality, and/or pH conditions) as a plant with one or more mutations described herein. A plant, plant part, and/or plant product of the present disclosure may have altered function (e.g., loss-of-function or reduced function) in the bZIP SIRT element, the bZIP transcription factor, or molecules regulated by the bZIP, as compared to a control plant, plant part, and/or plant product, when the plant or plant part of the present disclosure is grown under the same environmental conditions as the control plant or plant part.

In particular, function of the SIRT element in a plant, plant part, and/or plant product of the present disclosure may be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant, plant part, and/or plant product. Additionally, or alternatively, expression and/or function of SIRT element in a plant, plant part, and/or plant product may be reduced by at least 10%, 15%, 20%, 25%, 30%, 35%, 40%, 45%, 50%, 55%, 60%, 65%, 70%, 75%, 80%, 85%, 90%, 95%, or 100%, as compared to a control plant, plant part, and/or plant product. Expression of SIRT can be measured by any means known in the art for measuring peptide production, such as measuring mRNA levels or protein levels. Function (i.e., activity) of SIRT can be determined by measuring the resultant expression of the corresponding downstream gene. In specific embodiments, SIRT function can be determined by measuring the activity of the downstream bZIP transcription factor gene, such as bZIP1 or bZIP2.

In some embodiments, regulation by sucrose of expression or function of the bZIP transcription factor downstream of the SIRT element may be reduced or eliminated in a plant, plant part, and/or plant product of the present disclosure compared to a control plant, plant part, and/or plant product. In particular, regulation by sucrose of the bZIP expression or function in a plant, plant part, and/or plant product may be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant, plant part, and/or plant product. Additionally, or alternatively, the bZIP expression or function in a plant, plant part, and/or plant product may be reduced by at least 10%, 15%, 20%, 25%, 30%, 35%, 40%, 45%, 50%, 55%, 60%, 65%, 70%, 75%, 80%, 85%, 90%, 95%, or 100%, as compared to a control plant, plant part, and/or plant product.

In some embodiments, expression or function of the bZIP transcription factor downstream of the SIRT element may be increased in the absence or presence of sucrose in a plant, plant part, and/or plant product of the present disclosure compared to a control plant, plant part, and/or plant product. In particular, expression or function of the bZIP transcription factor in a plant, plant part, and/or plant product may be increased by about 1.1-100 fold, 10-100 fold, 20-100 fold, 30-100 fold, 40-100 fold, 50-100 fold, 60-100 fold, 70-100 fold, 80-100 fold, 20-90 fold, 30-90 fold, 40-90 fold, 50-90 fold, 60-90 fold, 70-90 fold, 1.1-10, 10-20 fold, 20-30 fold, 30-40 fold, 40-50 fold, 50-60 fold, 60-70 fold, 70-80 fold, 80-90 fold, 90-100 fold, or more than 100 fold, e.g., by about 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, or more than 100 fold in the absence or presence of sucrose as compared to a control plant or plant part. The expression or function of the bZIP transcription factor in a plant, plant part, and/or plant product can be increased by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant, plant part, and/or plant product. Additionally, or alternatively, expression or function the bZIP transcription factor in a plant or plant part of the present disclosure may be increased by at least 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, or 100 fold in the absence or presence of sucrose, as compared to a control plant or plant part. The expression or function of the bZIP transcription factor in a plant, plant part, and/or plant product may be increased by at least 10%, 15%, 20%, 25%, 30%, 35%, 40%, 45%, 50%, 55%, 60%, 65%, 70%, 75%, 80%, 85%, 90%, 95%, or 100% compared to a control.

It is well understood that alteration of transcription factor expression can in turn alter the expression of the target gene(s) of said transcription factor. In some embodiments, a plant, plant part, and/or plant product of the present disclosure comprises altered expression or function of a molecule downstream of, or regulated by, the bZIP transcription factor, such that the molecule comprises, for example: (i) increased baseline expression or function in the absence of sucrose; (ii) less reduction of the expression or the function in the presence of sucrose; and/or (iii) increase in the expression or the function in the presence of sucrose; compared to the control plant, plant part, and/or plant product.

In some embodiments, baseline expression or function of the molecule in the absence of sucrose is increased in a plant, plant part, and/or plant product of the present disclosure by about 1.1-100 fold, 10-100 fold, 20-100 fold, 30-100 fold, 40-100 fold, 50-100 fold, 60-100 fold, 70-100 fold, 80-100 fold, 20-90 fold, 30-90 fold, 40-90 fold, 50-90 fold, 60-90 fold, 70-90 fold, 1.1-10, 10-20 fold, 20-30 fold, 30-40 fold, 40-50 fold, 50-60 fold, 60-70 fold, 70-80 fold, 80-90 fold, 90-100 fold, or more than 100 fold, e.g., by about 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, or more than 100 fold, as compared to a control plant, plant part, and/or plant product. The baseline expression or function of the molecule in the absence of sucrose in a plant, plant part, and/or plant product of the present disclosure can be increased by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant, plant part, and/or plant product. Additionally, or alternatively, baseline expression or function of the molecule in the absence of sucrose is increased in a plant, plant part, and/or plant product by at least 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, or 100 fold, as compared to a control plant, plant part, and/or plant product. The baseline expression or function of the molecule in the absence of sucrose in a plant, plant part, and/or plant product may be increased by at least 10%, 15%, 20%, 25%, 30%, 35%, 40%, 45%, 50%, 55%, 60%, 65%, 70%, 75%, 80%, 85%, 90%, 95%, or 100% compared to a control. In some embodiments, baseline expression or function of the molecule in the absence of sucrose is increased in a plant, plant part, and/or plant product by about 1.1-25 fold (resulting in levels of about 110%-2500%) as compared to a control plant, plant part, and/or plant product.

In some embodiments, repression of expression or function of the molecule induced by sucrose (i.e., in the presence of sucrose compared to in the absence of sucrose) is reduced in a plant, plant part, and/or plant product of the present disclosure by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant, plant part, and/or plant product. Additionally, or alternatively, repression of expression or function of the molecule induced by sucrose (i.e., in the presence of sucrose compared to in the absence of sucrose) is reduced in a plant, plant part, and/or plant product by at least 10%, 15%, 20%, 25%, 30%, 35%, 40%, 45%, 50%, 55%, 60%, 65%, 70%, 75%, 80%, 85%, 90%, 95%, or 100%, as compared to a control plant, plant part, and/or plant product.

In some embodiments, expression or function of the molecule in the presence of sucrose in a plant, plant part, and/or plant product of the present disclosure is increased by about 1.1-100 fold, 10-100 fold, 20-100 fold, 30-100 fold, 40-100 fold, 50-100 fold, 60-100 fold, 70-100 fold, 80-100 fold, 20-90 fold, 30-90 fold, 40-90 fold, 50-90 fold, 60-90 fold, 70-90 fold, 1.1-10, 10-20 fold, 20-30 fold, 30-40 fold, 40-50 fold, 50-60 fold, 60-70 fold, 70-80 fold, 80-90 fold, 90-100 fold, or more than 100 fold, e.g., by about 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, or more than 100 fold, as compared to in the absence of sucrose (i.e., baseline). The expression or function of the molecule in the presence of sucrose in a plant, plant part, and/or plant product of the present disclosure can be increased by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to in the absence of sucrose. Additionally, or alternatively, expression or function of the molecule is increased in the presence of sucrose in a plant, plant part, and/or plant product by at least 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, or 100 fold, as compared to in the absence of sucrose (i.e., baseline). The expression or function of the molecule in the presence of sucrose in a plant, plant part, and/or plant product may be increased by at least 10%, 15%, 20%, 25%, 30%, 35%, 40%, 45%, 50%, 55%, 60%, 65%, 70%, 75%, 80%, 85%, 90%, 95%, or 100% compared to in the absence of sucrose. In some embodiments, expression or function of the molecule is increased in the presence of sucrose in a plant, plant part, and/or plant product by about 1.1-10 fold (resulting in levels of about 110%-1000%) as compared to the absence of sucrose (i.e., baseline).

In some embodiments, expression or function of the molecule in the presence of sucrose in a plant, plant part, and/or plant product of the present disclosure is increased by about 1.1-100 fold, 10-100 fold, 20-100 fold, 30-100 fold, 40-100 fold, 50-100 fold, 60-100 fold, 70-100 fold, 80-100 fold, 20-90 fold, 30-90 fold, 40-90 fold, 50-90 fold, 60-90 fold, 70-90 fold, 1.1-10, 10-20 fold, 20-30 fold, 30-40 fold, 40-50 fold, 50-60 fold, 60-70 fold, 70-80 fold, 80-90 fold, 90-100 fold, or more than 100 fold, e.g., by about 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, or more than 100 fold, as compared to a control plant, plant part, and/or plant product in the presence of sucrose. The expression or function of the molecule in the presence of sucrose in a plant, plant part, and/or plant product of the present disclosure can be increased by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant, plant part, and/or plant product in the presence of sucrose. Additionally, or alternatively, methods of the present disclosure can increase expression or function of the molecule in the presence of sucrose in a plant, plant part, and/or plant product by at least 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, or 100 fold, as compared to a control plant, plant part, and/or plant product in the presence of sucrose. The expression or function of the molecule in the presence of sucrose in a plant, plant part, and/or plant product may be increased by at least 10%, 15%, 20%, 25%, 30%, 35%, 40%, 45%, 50%, 55%, 60%, 65%, 70%, 75%, 80%, 85%, 90%, 95%, or 100% compared to a control in the presence of sucrose.

In some embodiments, the molecule regulated by the bZIP is asparagine synthase (ASN), proline dehydrogenase (PDH), sucrose phosphate synthase (SPS), sucrose-6′-phosphate phosphatase (SPP), calmodulin, or any other protein, peptide, carbohydrate, or enzyme that plays a role in carbohydrate metabolism or carbohydrate transport in plants. In specific embodiments, the molecule regulated by a bZIP transcription factor is sucrose.

Function or expression of an encoded bZIP SIRT peptide (e.g., SlbZIP1 SIRT, SlbZIP2 SIRT), a bZIP transcription factor, or molecules regulated by bZIP in a plant, plant part, and/or plant product can be determined by one or more standard methods known in the art. For example, expression of a SIRT peptide, a bZIP transcription factor, or a molecule regulated by the bZIP transcription factor can be measured by any means known in the art for measuring peptide production, such as measuring mRNA levels by PCR or protein levels by Western blot or ELISA.

Function (i.e., activity) of the bZIP transcription factor can be determined by measuring the expression or function of the downstream molecules, such as asparagine synthase (ASN), proline dehydrogenase (PDH), sucrose phosphate synthase (SPS), sucrose-6′-phosphate phosphatase (SPP), or calmodulin. In some embodiments, function of encoded bZIP SIRT peptide or bZIP transcription factor can be determined by introducing into a plant or plant part a DNA construct comprising, in operable linkage: a promoter that is functional in a plant cell; a nucleic acid sequence encoding the mutated bZIP upstream regulatory region (e.g., SIRT element); a nucleic acid sequence encoding the bZIP; and a reporter gene (e.g., GFP, an HA tag), and quantifying expression or function of the reporter gene linked to the mutated bZIP upstream regulatory region.

In some embodiments, function of molecules regulated by bZIP (e.g., asparagine synthase (ASN), proline dehydrogenase (PDH), sucrose phosphate synthase (SPS), sucrose-6′-phosphate phosphatase (SPP), or calmodulin) may be determined by assays to determine enzymatic activity. Details of such procedure has been provided elsewhere in the present disclosure.

C. Plants with Increased Sugar/Total Soluble Solids Content

Plants or plant parts of the present disclosure, comprising one or more mutations in the upstream regulatory region of the bZIP, or plant products (e.g., plant extract, plant concentrate, plant powder, plant protein, and plant biomass) obtained from such plants may have increased levels of total soluble solids (TSS), total sugar, sucrose, glucose, fructose, galactose, maltose, and/or lactose, as compared to a control plant, plant part, or plant product. Thus, a mutant plant, plant part, or plant product of the present disclosure can have improved flavor characteristics compared to a control plant, plant part, or plant product.

A control plant or plant part can be a plant or plant part to which one or more mutations has not be introduced by the methods provided herein. Thus, a control plant or plant part may express a wild-type (WT) SIRT element. A control plant of the present disclosure may be grown under the same environmental conditions (e.g., same or similar temperature, humidity, air quality, soil quality, water quality, and/or pH conditions) as a plant with one or more mutations described herein. A plant, plant part, and/or plant product of the present disclosure may have increased levels of total soluble solids (TSS), total sugar, sucrose, glucose, fructose, galactose, maltose, lactose, and/or improved flavor characteristics, as compared to a control plant, plant part, and/or plant product, when the plant or plant part of the present disclosure is grown under the same environmental conditions as the control plant or plant part. Improved flavor characteristics of plants, plant parts, and/or plant products of the present disclosure can result from increased levels of total soluble solids (TSS), total sugar, sucrose, glucose, fructose, galactose, maltose, and/or lactose in such plants, plant parts, or plant products.

In some embodiments, TSS levels in a plant, plant part, and/or plant product of the present invention are increased by about 1.1-100 fold, 10-100 fold, 20-100 fold, 30-100 fold, 40-100 fold, 50-100 fold, 60-100 fold, 70-100 fold, 80-100 fold, 20-90 fold, 30-90 fold, 40-90 fold, 50-90 fold, 60-90 fold, 70-90 fold, 1.1-10, 10-20 fold, 20-30 fold, 30-40 fold, 40-50 fold, 50-60 fold, 60-70 fold, 70-80 fold, 80-90 fold, 90-100 fold, or more than 100 fold, e.g., by about 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, or more than 100 fold, as compared to a control plant, plant part, and/or plant product. Additionally, or alternatively, methods of the present disclosure can increase TSS levels in a plant, plant part, and/or plant product by at least 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, or 100 fold, as compared to a control plant, plant part, and/or plant product. TSS levels in a plant, plant part, and/or plant product of the present disclosure can be increased by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant, plant part, and/or plant product. The TSS levels in a plant, plant part, and/or plant product may be increased by at least 10%, 15%, 20%, 25%, 30%, 35%, 40%, 45%, 50%, 55%, 60%, 65%, 70%, 75%, 80%, 85%, 90%, 95%, or 100% compared to a control. TSS levels can be measured by any methods known in the art. For example, TSS can be determined by using spectroscopy (near infrared spectroscopy), refractometry, or any method to measure or estimate the amount of TSS in a fruit, plant, or population of fruits or plants.

In some embodiments, total sugar levels in a plant, plant part, and/or plant product of the present invention are increased by about 1.1-100 fold, 10-100 fold, 20-100 fold, 30-100 fold, 40-100 fold, 50-100 fold, 60-100 fold, 70-100 fold, 80-100 fold, 20-90 fold, 30-90 fold, 40-90 fold, 50-90 fold, 60-90 fold, 70-90 fold, 1.1-10, 10-20 fold, 20-30 fold, 30-40 fold, 40-50 fold, 50-60 fold, 60-70 fold, 70-80 fold, 80-90 fold, 90-100 fold, or more than 100 fold, e.g., by about 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, or more than 100 fold, as compared to a control plant, plant part, and/or plant product. Additionally, or alternatively, methods of the present disclosure can increase total sugar levels in a plant, plant part, and/or plant product by at least 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, or 100 fold, as compared to a control plant, plant part, and/or plant product. Total sugar levels in a plant, plant part, and/or plant product of the present disclosure can be increased by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant, plant part, and/or plant product. Total sugar levels in a plant, plant part, and/or plant product may be increased by at least 10%, 15%, 20%, 25%, 30%, 35%, 40%, 45%, 50%, 55%, 60%, 65%, 70%, 75%, 80%, 85%, 90%, 95%, or 100% compared to a control. Sugar levels can be measured by any methods known in the art. For example, sugar levels can be determined by using spectroscopy (near infrared spectroscopy), refractometry, or any method to measure or estimate the amount of sugar in a fruit, plant, or population of fruits or plants.

In some embodiments, sucrose, glucose, fructose, galactose, maltose, and/or lactose levels in a plant, plant part, and/or plant product of the present invention are increased by about 1.1-100 fold, 10-100 fold, 20-100 fold, 30-100 fold, 40-100 fold, 50-100 fold, 60-100 fold, 70-100 fold, 80-100 fold, 20-90 fold, 30-90 fold, 40-90 fold, 50-90 fold, 60-90 fold, 70-90 fold, 1.1-10, 10-20 fold, 20-30 fold, 30-40 fold, 40-50 fold, 50-60 fold, 60-70 fold, 70-80 fold, 80-90 fold, 90-100 fold, or more than 100 fold, e.g., by about 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, or more than 100 fold, as compared to a control plant, plant part, and/or plant product. Additionally, or alternatively, methods of the present disclosure can increase sucrose, glucose, fructose, galactose, maltose, and/or lactose levels in a plant, plant part, and/or plant product by at least 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, or 100 fold, as compared to a control plant, plant part, and/or plant product. In specific embodiments, the level of sucrose is increased by about 1.1-fold to about 25-fold in a plant having a mutation (e.g., deletion) in a SIRT element of a bZIP1 or bZIP2 coding sequence when compared to a control plant. Sucrose, glucose, fructose, galactose, maltose, and/or lactose levels in a plant, plant part, and/or plant product of the present disclosure can be increased by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant, plant part, and/or plant product. Sucrose, glucose, fructose, galactose, maltose, and/or lactose levels in a plant, plant part, and/or plant product may be increased by at least 10%, 15%, 20%, 25%, 30%, 35%, 40%, 45%, 50%, 55%, 60%, 65%, 70%, 75%, 80%, 85%, 90%, 95%, or 100% compared to a control. The level of any specific sugar can be measured by any standard method in the art including GC, MS, chromatography, or any other methods of measuring or estimating the amount of a specific sugar in a plant sample.

The amount or level of total soluble solids (TSS), total sugar, sucrose, glucose, fructose, galactose, maltose, and/or lactose in a plant, plant part (e.g., fruit), and/or plant product (e.g., plant extract, plant concentrate, plant powder, plant protein, or plant biomass) can be determined by one or more standard methods known in the art. In some embodiments, amount or level of total soluble solids (TSS), total sugar, sucrose, glucose, fructose, galactose, maltose, and/or lactose in a plant, plant part, plant extract, plant concentrate, plant powder, plant protein, and/or plant biomass is determined by solid-phase extraction (SPE), solid-phase micro-extraction (SPME), high performance liquid chromatography (HPLC), gas chromatography mass spectrometry (GCMS), and/or enzymatic assay.

Also provided herein are plant parts (e.g., juice, pulp, seed, fruit, flowers, nectar, embryos, pollen, ovules, leaves, stems, branches, bark, kernels, ears, cobs, husks, stalks, roots, root tips, anthers, etc.), plant extract (e.g., sweetener, antioxidants, alkaloids, etc.), plant concentrate (e.g., whole plant concentrate or plant part concentrate), plant powder (e.g., formulated powder, such as formulated plant part powder (e.g., seed flour)), and plant biomass (e.g., dried biomass, such as crushed and/or powdered biomass) obtained from plants of the present disclosure. Also provided herein are seeds, such as a representative sample of seeds, from a plant of the present disclosure. A plant or plant part of the present disclosure can be a crop plant or part of a crop plant.

Also provided herein are food and/or beverage products containing plant compositions (e.g., plant parts, plant extract, plant concentrate, plant powder, plant protein, and plant biomass) described herein, such as plant compositions derived from the plants or plant parts of the present disclosure. Such food and/or beverage products include, without limitation, shakes, juices, health drinks, tomato sauce, tomato paste, ketchup, and condiments. A food and/or beverage product that contains plant compositions obtained from plants or plant parts of the present disclosure can have increased sugar content and improved flavor characteristics, compared to a similar or comparable food and/or beverage product that contains plant compositions obtained from a control plant or plant part.

Plant parts (e.g., seeds) and plant products (e.g., plant biomass, seed compositions, protein compositions, food and/or beverage products) produced by the methods provided herein can be meant for consumption by agricultural animals or for use as feed in an agriculture or aquaculture system. In specific embodiments, plant parts and plant products produced according to the methods provided herein include animal feed (e.g., roughages—forage, hay, silage; concentrates—cereal grains, soybean cake) intended for consumption by bovine, porcine, poultry, lambs, goats, or any other agricultural animal. In some embodiments, plant parts and plant products produced according to the methods include aquaculture feed for any type of fish or aquatic animal in a farmed or wild environment including, without limitation, trout, carp, catfish, salmon, tilapia, crab, lobster, shrimp, oysters, clams, mussels, and scallops.

### IV. Altering bZIP Function to Increase Sugar Content in Plants

In some aspects, provided herein are methods for altering function of a basic region/leucine zipper motif (bZIP) transcription factor gene or a homolog thereof in a plant or plant part. In some aspects, the methods comprise introducing one or more insertions, substitutions, or deletions into the upstream open reading frame (uORF) of the bZIP transcription factor gene or the homolog thereof in the plant or plant part, wherein the uORF comprises a sucrose induced repression of translation (SIRT) element. In some aspects, the methods of the present disclosure alter function of the SIRT element, and increase sugar content of the plant or plant part relative to a control plant or plant part not subjected to the methods.

In some embodiments, methods of the present disclosure can reduce function (i.e., activity) of the bZIP SIRT element in a plant or plant part by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part. Additionally, or alternatively, methods of the present disclosure can reduce expression and/or function of SIRT element in a plant or plant part by at least 10%, 15%, 20%, 25%, 30%, 35%, 40%, 45%, 50%, 55%, 60%, 65%, 70%, 75%, 80%, 85%, 90%, 95%, or 100%, as compared to a control plant or plant part.

In some embodiments, the methods of the present disclosure can reduce or eliminate regulation by sucrose of expression or function of the bZIP transcription factor downstream of the SIRT element. In particular, methods of the present disclosure can decrease regulation by sucrose of the bZIP function in a plant or plant part by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part. Additionally, or alternatively, methods of the present disclosure can reduce or eliminate regulation by sucrose of expression or function of the bZIP in a plant or plant part by at least 10%, 15%, 20%, 25%, 30%, 35%, 40%, 45%, 50%, 55%, 60%, 65%, 70%, 75%, 80%, 85%, 90%, 95%, or 100%, as compared to a control plant or plant part.

In some embodiments, the methods of the present disclosure can increase expression or function of the bZIP transcription factor downstream of the SIRT element in the absence or presence of sucrose. In particular, methods of the present disclosure can increase expression or function of the bZIP transcription factor by about 1.1-100 fold, 10-100 fold, 20-100 fold, 30-100 fold, 40-100 fold, 50-100 fold, 60-100 fold, 70-100 fold, 80-100 fold, 20-90 fold, 30-90 fold, 40-90 fold, 50-90 fold, 60-90 fold, 70-90 fold, 1.1-10, 10-20 fold, 20-30 fold, 30-40 fold, 40-50 fold, 50-60 fold, 60-70 fold, 70-80 fold, 80-90 fold, 90-100 fold, or more than 100 fold, e.g., by about 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, or more than 100 fold in the absence or presence of sucrose, as compared to a control plant or plant part. Additionally, or alternatively, methods of the present disclosure can increase expression or function the bZIP transcription factor in a plant or plant part by at least 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold in the absence or presence of sucrose, as compared to a control plant or plant part. Expression or function of the bZIP transcription factor in a plant or plant part can be increased by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part. The expression or function of the bZIP transcription factor in a plant or plant part may be increased by at least 10%, 15%, 20%, 25%, 30%, 35%, 40%, 45%, 50%, 55%, 60%, 65%, 70%, 75%, 80%, 85%, 90%, 95%, or 100% compared to a control.

It is well understood that alteration of transcription factor expression can in turn alter the expression of the target gene(s) of said transcription factor (Hiratsu et al. (2003) Plant J 34:733-739). In some embodiments, the methods of the present disclosure alter expression or function of a molecule downstream of, or regulated by, the bZIP, such that the molecule comprises, for example: (i) increased baseline expression or function in the absence of sucrose; (ii) less reduction of the expression or the function in the presence of sucrose; and/or (iii) increase in the expression or the function in the presence of sucrose; compared to the control plant or plant part.

In some embodiments, baseline expression or function of the molecule in the absence of sucrose is increased by about 1.1-100 fold, 10-100 fold, 20-100 fold, 30-100 fold, 40-100 fold, 50-100 fold, 60-100 fold, 70-100 fold, 80-100 fold, 20-90 fold, 30-90 fold, 40-90 fold, 50-90 fold, 60-90 fold, 70-90 fold, 1.1-10, 10-20 fold, 20-30 fold, 30-40 fold, 40-50 fold, 50-60 fold, 60-70 fold, 70-80 fold, 80-90 fold, 90-100 fold, or more than 100 fold, e.g., by about 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, or more than 100 fold, as compared to a control plant or plant part. Additionally, or alternatively, methods of the present disclosure can increase baseline expression or function of the molecule in the absence of sucrose in a plant or plant part by at least 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, or 100 fold, as compared to a control plant or plant part. Baseline expression or function of the molecule in the absence of sucrose in a plant or plant part can be increased by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part. The baseline expression or function of the molecule in the absence of sucrose in a plant or plant part may be increased by at least 10%, 15%, 20%, 25%, 30%, 35%, 40%, 45%, 50%, 55%, 60%, 65%, 70%, 75%, 80%, 85%, 90%, 95%, or 100% compared to a control.

In some embodiments, the molecule regulated by the bZIP is asparagine synthase (ASN), proline dehydrogenase (PDH), sucrose phosphate synthase (SPS), sucrose-6′-phosphate phosphatase (SPP), calmodulin, or sucrose.

In some embodiments, repression of expression or function of the molecule induced by sucrose (i.e., in the presence of sucrose compared to in the absence of sucrose) is reduced in a plant or plant part by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part. Additionally, or alternatively, methods of the present disclosure can reduce repression of expression or function of the molecule induced by sucrose (i.e., in the presence of sucrose compared to in the absence of sucrose) in a plant or plant part by at least 10%, 15%, 20%, 25%, 30%, 35%, 40%, 45%, 50%, 55%, 60%, 65%, 70%, 75%, 80%, 85%, 90%, 95%, or 100%, as compared to a control plant or plant part. In some embodiments, the molecule regulated by the bZIP is asparagine synthase (ASN), proline dehydrogenase (PDH), sucrose phosphate synthase (SPS), sucrose-6′-phosphate phosphatase (SPP), calmodulin, or sucrose.

In some embodiments, expression or function of the molecule in the presence of sucrose is increased by about 1.1-100 fold, 10-100 fold, 20-100 fold, 30-100 fold, 40-100 fold, 50-100 fold, 60-100 fold, 70-100 fold, 80-100 fold, 20-90 fold, 30-90 fold, 40-90 fold, 50-90 fold, 60-90 fold, 70-90 fold, 1.1-10, 10-20 fold, 20-30 fold, 30-40 fold, 40-50 fold, 50-60 fold, 60-70 fold, 70-80 fold, 80-90 fold, 90-100 fold, or more than 100 fold, e.g., by about 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, or more than 100 fold, as compared to in the absence of sucrose (i.e., baseline). Additionally, or alternatively, methods of the present disclosure can increase expression or function of the molecule in the presence of sucrose in a plant or plant part by at least 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, or 100 fold, as compared to in the absence of sucrose (i.e., baseline). Expression or function of the molecule in the presence of sucrose in a plant or plant part can be increased by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to in the absence of sucrose. The expression or function of the molecule in the presence of sucrose in a plant or plant part may be increased by at least 10%, 15%, 20%, 25%, 30%, 35%, 40%, 45%, 50%, 55%, 60%, 65%, 70%, 75%, 80%, 85%, 90%, 95%, or 100% compared to in the absence of sucrose. In some embodiments, the molecule regulated by the bZIP is asparagine synthase (ASN), proline dehydrogenase (PDH), sucrose phosphate synthase (SPS), sucrose-6′-phosphate phosphatase (SPP), calmodulin, or sucrose.

In some embodiments, expression or function of the molecule in the presence of sucrose is increased by about 1.1-100 fold, 10-100 fold, 20-100 fold, 30-100 fold, 40-100 fold, 50-100 fold, 60-100 fold, 70-100 fold, 80-100 fold, 20-90 fold, 30-90 fold, 40-90 fold, 50-90 fold, 60-90 fold, 70-90 fold, 1.1-10, 10-20 fold, 20-30 fold, 30-40 fold, 40-50 fold, 50-60 fold, 60-70 fold, 70-80 fold, 80-90 fold, 90-100 fold, or more than 100 fold, e.g., by about 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, or 100 fold, or more than 100 fold, as compared to a control plant or plant part in the presence of sucrose. Additionally, or alternatively, methods of the present disclosure can increase expression or function of the molecule in the presence of sucrose in a plant or plant part by at least 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, as compared to a control plant or plant part in the presence of sucrose. Expression or function of the molecule in the presence of sucrose in a plant or plant part can be increased by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part in the presence of sucrose. The expression or function of the molecule in the presence of sucrose in a plant or plant part may be increased by at least 10%, 15%, 20%, 25%, 30%, 35%, 40%, 45%, 50%, 55%, 60%, 6%, 70%, 75%, 80%, 85%, 90%, 95%, or 100% compared to a control in the presence of sucrose. In some embodiments, the molecule regulated by the bZIP is asparagine synthase (ASN), proline dehydrogenase (PDH), sucrose phosphate synthase (SPS), sucrose-6′-phosphate phosphatase (SPP), calmodulin, or sucrose.

In some embodiments, the methods of the present disclosure increase one or more of total soluble solids (TSS), total sugar, sucrose, glucose, fructose, galactose, maltose, and lactose levels in a fruit of the plant or plant part relative to the control plant or plant part.

In some embodiments, TSS levels in a plant or plant part are increased by about 1.1-100 fold, 10-100 fold, 20-100 fold, 30-100 fold, 40-100 fold, 50-100 fold, 60-100 fold, 70-100 fold, 80-100 fold, 20-90 fold, 30-90 fold, 40-90 fold, 50-90 fold, 60-90 fold, 70-90 fold, 1.1-10, 10-20 fold, 20-30 fold, 30-40 fold, 40-50 fold, 50-60 fold, 60-70 fold, 70-80 fold, 80-90 fold, 90-100 fold, or more than 100 fold, e.g., by about 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, or more than 100 fold, as compared to a control plant or plant part. Additionally, or alternatively, methods of the present disclosure can increase TSS levels in a plant or plant part by at least 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, or 100 fold, as compared to a control plant or plant part. TSS levels in a plant, plant part, and/or plant product of the present disclosure can be increased by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant, plant part, and/or plant product. The TSS levels in a plant, plant part, and/or plant product may be increased by at least 10%, 15%, 20%, 25%, 30%, 35%, 40%, 45%, 50%, 55%, 60%, 65%, 70%, 75%, 80%, 85%, 90%, 95%, or 100% compared to a control.

In some embodiments, total sugar levels in a plant or plant part are increased by about 1.1-100 fold, 10-100 fold, 20-100 fold, 30-100 fold, 40-100 fold, 50-100 fold, 60-100 fold, 70-100 fold, 80-100 fold, 20-90 fold, 30-90 fold, 40-90 fold, 50-90 fold, 60-90 fold, 70-90 fold, 1.1-10, 10-20 fold, 20-30 fold, 30-40 fold, 40-50 fold, 50-60 fold, 60-70 fold, 70-80 fold, 80-90 fold, 90-100 fold, or more than 100 fold, e.g., by about 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, or more than 100 fold, as compared to a control plant or plant part. Additionally, or alternatively, methods of the present disclosure can increase total sugar levels in a plant or plant part by at least 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, or 100 fold, as compared to a control plant or plant part. Total sugar levels in a plant, plant part, and/or plant product of the present disclosure can be increased by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant, plant part, and/or plant product. The total sugar levels in a plant, plant part, and/or plant product may be increased by at least 10%, 15%, 20%, 25%, 30%, 35%, 40%, 45%, 50%, 55%, 60%, 65%, 70%, 75%, 80%, 85%, 90%, 95%, or 100% compared to a control.

In some embodiments, sucrose, glucose, fructose, galactose, maltose, and/or lactose levels in a plant or plant part are increased by about 1.1-100 fold, 10-100 fold, 20-100 fold, 30-100 fold, 40-100 fold, 50-100 fold, 60-100 fold, 70-100 fold, 80-100 fold, 20-90 fold, 30-90 fold, 40-90 fold, 50-90 fold, 60-90 fold, 70-90 fold, 1.1-10, 10-20 fold, 20-30 fold, 30-40 fold, 40-50 fold, 50-60 fold, 60-70 fold, 70-80 fold, 80-90 fold, 90-100 fold, or more than 100 fold, e.g., by about 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, or more than 100 fold, as compared to a control plant or plant part. Additionally, or alternatively, methods of the present disclosure can increase sucrose, glucose, fructose, galactose, maltose, and/or lactose levels in a plant or plant part by at least 1.1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, or 100 fold, as compared to a control plant or plant part. Sucrose, glucose, fructose, galactose, maltose, and/or lactose levels in a plant, plant part, and/or plant product of the present disclosure can be increased by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant, plant part, and/or plant product. The sucrose, glucose, fructose, galactose, maltose, and/or lactose levels in a plant, plant part, and/or plant product may be increased by at least 10%, 15%, 20%, 25%, 30%, 35%, 40%, 45%, 50%, 55%, 60%, 65%, 70%, 75%, 80%, 85%, 90%, 95%, or 100% compared to a control.

Increasing sugar and/or TSS content can improve flavor characteristics of a plant or plant part, and/or can improve flavor characteristics in a plant extract, plant concentrate, plant powder, plant protein, or plant biomass obtained from such plant or plant part. Thus, also provided herein are methods for improving flavor characteristics in plant, plant part, plant extract, plant concentrate, plant powder, plant protein, and/or plant biomass.

Function or expression of SIRT, bZIP, and molecules regulated by bZIP may be altered in accordance with the disclosure set forth below.

A. Introducing Mutation to Upstream Regulatory Region of bZIP

In some aspects, altering expression or function of the bZIP SIRT element comprises introducing one or more insertions, substitutions, or deletions into the upstream regulatory region (e.g., 5′UTR, uORF, SIRT element) of the bZIP transcription factor gene in a plant or plant part. In some embodiments a control plant or plant part described herein (e.g., a plant or plant part to which mutations can be introduced by the methods of the present disclosure) may have a SIRT peptide (e.g., SlbZIP1 SIRT) that comprises an amino acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence set forth in SEQ ID NO: 5 or 6. For example, a control plant or plant part described herein may have a SIRT peptide that comprises the amino acid sequence of SEQ ID NO: 5 or 6.

A control plant or plant part described herein (e.g., a plant or plant part to which mutations may be introduced by the methods of the present disclosure) may comprise a SIRT (e.g., SlbZIP1 SIRT) nucleic acid sequence that encodes a SIRT peptide. A SIRT nucleic acid sequence may comprise at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence set forth in SEQ ID NO: 3 or 4. For example, a control plant or plant part described herein may have a SIRT gene that comprises the nucleic acid sequence of SEQ ID NO: 3 or 4. A plant or plant part described herein may comprise 1-6, 2-4, 3-4, 2-5, or 3-5 (e.g., 1, 2, 3, 4, 5, or 6) copies of SIRT element (e.g., SlbZIP1 SIRT). In particular, a plant or plant part described herein may comprise at least 2 genes encoding a SIRT peptide, such as 2 genes that have less than 100% (e.g., less than 100%, 99%, 98%, 97%, 96%, 95%, 94%, 93%, 92%, 91%, 90%, 89%, 88%, 87%, 86%, or 85%) sequence identity.

In some embodiments, provided herein are methods for altering the expression or function of the bZIP transcription factor gene in a plant or plant part by introducing one or more mutations in a regulatory element (e.g., 5′ UTR, uORF, SIRT element) that regulates the expression of the bZIP (e.g., SlbZIP1) gene. The methods of the present disclosure may comprise introducing one or more (e.g., about 1, 2, 3, 4, 5, 6, 7, 8, 9, 10, 11, 12, 13, 14, 15, 16, 17, 18, 19, 20, or more) insertions, substitutions or deletions in a bZIP 5′UTR, a bZIP uORF, or a bZIP SIRT element (i.e., a nucleic acid sequence representing a bZIP SIRT element, or a gene encoding a bZIP SIRT peptide) in the genome of the plant cell or plant part.

In particular, introducing mutation to the genes by the methods of the present disclosure may comprise one or more insertions, substitutions or deletions in a nucleotide region corresponding to the 5′UTR of the bZIP transcription factor gene, e.g., uORF of the bZIP transcription factor gene or the bZIP SIRT element. For example, introducing mutation to the regulatory element of the bZIP transcription factor gene in the genome of a plant cell or plant part by the methods of the present disclosure may comprise one or more insertions, substitutions or deletions of about 1-84, 2-84, 3-84, 4-84, 5-84, 6-84, 7-84, 8-84, 9-84, 10-84, or 11-84 (e.g., about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, or 84) nucleotides of bZIP 5′UTR, bZIP uORF, or bZIP SIRT in the genome of a plant cell or plant part. In particular, gene editing by the methods of the present disclosure may comprise one or more insertions, substitutions or deletions of about 7-9 nucleotides in the bZIP 5′UTR, bZIP uORF, or bZIP SIRT. The mutation (e.g., insertion, substitution, or deletion) may be an in-frame mutation in the bZIP SIRT element or an out-of-frame mutation in the bZIP SIRT element.

The one or more insertions, substitutions or deletions in the bZIP upstream regulatory region (e.g., bZIP SIRT element) introduced by the methods of the present disclosure may be in a nucleotide region that comprises a nucleic acid sequence having at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence of SEQ ID NO: 3 or 4.

For example, the one or more insertions, substitutions or deletions in in the bZIP upstream regulatory region (e.g., bZIP SIRT element) introduced by the methods of the present disclosure may comprise a deletion of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, or 84 nucleotides in a nucleotide region that comprises the nucleic acid sequence of SEQ ID NO: 3 or 4. In particular, the one or more insertions, substitutions or deletions in the bZIP upstream regulatory region (e.g., bZIP SIRT element) may comprise a deletion of about 7-9 nucleotides. In some embodiments, the deletion is a deletion of nucleotide 6 through 13 of SEQ ID NO: 3, resulting in a sequence set forth in SEQ ID: 7; a deletion of nucleotide 53 through 60 of SEQ ID NO: 3, resulting in a sequence set forth in SEQ ID: 8; a deletion of nucleotide 53 through 61 of SEQ ID NO: 3, resulting in a sequence set forth in SEQ ID: 9; or a deletion of nucleotide 261 through 268 of SEQ ID NO: 1, resulting in a deletion of nucleotide 69 through 75 of SEQ ID NO: 3 and a sequence set forth in SEQ ID: 10. In some embodiments, the mutated SIRT element shares at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) identity with a sequence selected from the group consisting of SEQ ID No: 7-10.

In some embodiments, the one or more insertions, substitutions or deletions in the bZIP 5′UTR, the bZIP uORF, or the bZIP SIRT nucleic acid sequence introduced by the methods of the present disclosure may produce a peptide comprising one or more amino acid changes (e.g., 1, 2, 3, 4, 5, 6, 7, 8, 9, 10, 11, 12, 13, 14, 15, 16, 17, 18, 19, 20, 21, 22, 23, 24, 25, 26, 27, or 28 amino acid changes) in the amino acid sequence that comprises the amino acid sequence of SEQ ID NO: 5 or 6 in the plant or plant part. In particular, the one or more insertions, substitutions or deletions in the bZIP upstream regulatory region (e.g., the bZIP SIRT element) may comprise 1, 2, or 3 amino acid changes in a SIRT peptide sequence. In some embodiments, the change comprises one or more of M1L, M4L, and M10L compared to the amino acid sequence of SEQ ID NO: 5. In some embodiments, the mutated SIRT element shares at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) identity with the amino acid sequence of SEQ ID NO: 11.

A bZIP 5′UTR, a bZIP uORF, or a bZIP SIRT element mutated by the methods of the present disclosure (e.g., by deletion of about 7-9 nucleotides in the SIRT element, or by changes of about 1, 2, or 3 amino acids in the SIRT peptide sequence) may produce loss-of-function or reduced function of the SIRT element, as compared to a wild-type SIRT element (i.e., SIRT element that has not been edited by the methods of the present disclosure). A mutated bZIP SIRT element may be found in plants or plant parts to which one or more mutations have been introduced by the methods of the present disclosure. A mutated bZIP SIRT element can also be found in plant parts (e.g., juice, pulp, seed, fruit, flowers, nectar, embryos, pollen, ovules, leaves, stems, branches, bark, kernels, ears, cobs, husks, stalks, roots, root tips, anthers, etc.), plant extract (e.g., sweetener, antioxidants, alkaloids, etc.), plant concentrate (e.g., whole plant concentrate or plant part concentrate), plant powder (e.g., formulated powder, such as formulated plant part powder (e.g., seed flour)), and plant biomass (e.g., dried biomass, such as crushed and/or powdered biomass) obtained from such plants.

Methods disclosed herein are not limited to certain techniques of mutagenesis. Any method of creating a change in a nucleic acid of a plant can be used in conjunction with the disclosed invention, including the use of chemical mutagens (e.g. methanesulfonate, sodium azide, aminopurine, etc.), genome/gene editing techniques (e.g. CRISPR-like technologies, TALENs, zinc finger nucleases, and meganucleases), ionizing radiation (e.g. ultraviolet and/or gamma rays) temperature alterations, long-term seed storage, tissue culture conditions, targeting induced local lesions in a genome, sequence-targeted and/or random recombinases, etc. It is anticipated that new methods of creating a mutation in a nucleic acid of a plant will be developed and yet fall within the scope of the claimed invention when used with the teachings described herein.

Similarly, the embodiments disclosed herein are not limited to certain methods of introducing nucleic acids into a plant and are not limited to certain forms or structures that the introduced nucleic acids take. Any method of transforming a cell of a plant described herein with nucleic acids are also incorporated into the teachings of this innovation, and one of ordinary skill in the art will realize that the use of particle bombardment (e.g. using a gene-gun), Agrobacterium infection and/or infection by other bacterial species capable of transferring DNA into plants (e.g., Ochrobactrum sp., Ensifer sp., Rhizobium sp.), viral infection, and other techniques can be used to deliver nucleic acid sequences into a plant described herein. Methods disclosed herein are not limited to any size of nucleic acid sequences that are introduced, and thus one could introduce a nucleic acid comprising a single nucleotide (e.g. an insertion) into a nucleic acid of the plant and still be within the teachings described herein. Nucleic acids introduced in substantially any useful form, for example, on supernumerary chromosomes (e.g. B chromosomes), plasmids, vector constructs, additional genomic chromosomes (e.g. substitution lines), and other forms is also anticipated. It is envisioned that new methods of introducing nucleic acids into plants and new forms or structures of nucleic acids will be discovered and yet fall within the scope of the claimed invention when used with the teachings described herein.

Methods disclosed herein include conferring desired traits (e.g., increased sucrose content) to plants, for example, by mutating sequences of a plant, introducing nucleic acids into plants, using plant breeding techniques and various crossing schemes, etc. These methods are not limited as to certain mechanisms of how the plant exhibits and/or expresses the desired trait. In certain nonlimiting embodiments, the trait is conferred to the plant by introducing a nucleic acid sequence (e.g. using plant transformation methods) that encodes production of a certain protein by the plant. In certain nonlimiting embodiments, the desired trait is conferred to a plant by causing a null mutation in the plant's genome (e.g. when the desired trait is reduced expression or no expression of a certain trait). In certain nonlimiting embodiments, the desired trait is conferred to a plant by crossing two plants to create offspring that express the desired trait. It is expected that users of these teachings will employ a broad range of techniques and mechanisms known to bring about the expression of a desired trait in a plant. Thus, as used herein, conferring a desired trait to a plant is meant to include any process that causes a plant to exhibit a desired trait, regardless of the specific techniques employed.

In certain embodiments, a user can combine the teachings herein with high-density molecular marker profiles spanning substantially the entire genome of a plant to estimate the value of selecting certain candidates in a breeding program in a process commonly known as genome selection.

### B. Transformation of Plants

Provided herein are methods for transforming plants or plant parts by introducing into the plants or plant parts a system, e.g., a gene editing system, or a construct for introducing one or more mutations (e.g., insertions, substitutions, or deletions) to the upstream regulatory region of bZIP (e.g., bZIP SIRT element). The term “transform” or “transformation” refers to any method used to introduce polypeptides or polynucleotides into plant cells. For purpose of the present disclosure, the transformation can be “stable transformation”, wherein the transformation construct (e.g., a construct comprising a gRNA and/or a gene encoding a nuclease for use in the methods of the present invention) is introduced into a host (e.g., a host plant, plant part, plant cell, etc.) and integrates into the genome of the host and is capable of being inherited by the progeny thereof; or “transient transformation”, wherein the transformation construct (e.g., a construct comprising a gRNA and/or a gene encoding a nuclease for use in the methods of the present invention) is introduced into a host (e.g., a host plant, plant part, plant cell, etc.) and expressed temporarily. The methods disclosed herein can also be used for insertion of heterologous genes and/or modification of native plant gene expression to achieve desirable plant traits, e.g., increased sugar content.

Gene editing of uORF can effectively regulate translation of genes encoded by downstream ORFs in plants. Modulation of the upstream regulatory region of the bZIP to alter the bZIP expression or function may be achieved through the use of precise genome-editing technologies to modulate the expression of the endogenous sequence. In this manner, a nucleic acid sequence can be inserted, substituted, or deleted proximal to or within a native plant sequence corresponding to a bZIP SIRT element through the use of methods available in the art. Such methods include, but are not limited to, use of meganucleases designed against the plant genomic sequence of interest (D'Halluin et al (2013) Plant Biotechnol J 11: 933-941); CRISPR-Cas9, CRISPR-Cas12a (Cpf1), transcription activator-like effector nucleases (TALENs), zinc finger nucleases (ZFNs), and other technologies for precise editing of genomes [Feng et al. (2013) Cell Research 23:1229-1232, Podevin et al. (2013) Trends Biotechnology 31: 375-383, Wei et al. (2013) J Gen Genomics 40:281-289, Zhang et al (2013) WO 2013/026740, Zetsche et al. (2015) Cell 163:759-771, U.S. Provisional Patent Application 62/295,325]; N. gregoryi Argonaute-mediated DNA insertion (Gao et al. (2016) Nat Biotechnol doi:10.1038/nbt.3547); Cre-lox site-specific recombination (Dale et al. (1995) Plant J 7:649-659; Lyznik, et al. (2007) Transgenic Plant J 1:1-9; FLP-FRT recombination (Li et al. (2009) Plant Physiol 151:1087-1095); Bxb1-mediated integration (Yau et al. (2011) Plant J 701:147-166); zinc-finger mediated integration (Wright et al. (2005) Plant J 44:693-705); Cai et al. (2009) Plant Mol Biol 69:699-709); and homologous recombination (Lieberman-Lazarovich and Levy (2011) Methods Mol Biol 701: 51-65; Puchta (2002) Plant Mol Biol 48:173-182).

In some aspects, inserting, substituting, or deleting one or more nucleotides at a precise location of interest in the upstream regulatory region of the bZIP (e.g., bZIP SIRT element) may be achieved using a meganuclease or other suitable nuclease system designed to target the genomic sequence of interest. Without wishing to be bound by theory, a nuclease system can be used to achieve insertion, substitution, or deletion of genetic elements at a predefined genomic locus by causing a double-strand break at said predefined genomic locus and, optionally, providing an appropriate DNA template for insertion. This strategy is well-understood and has been demonstrated previously to insert a transgene at a predefined location in the cotton genome (D'Halluin et al. (2013) Plant Biotechnol J 11: 933-941). For example, a Cas12a (Cpf1) endonuclease coupled with a guide RNA (gRNA) designed against the genomic sequence of interest (i.e., bZIP SIRT element) can be used (i.e., a CRISPR-Cas12a system). Alternatively, a Cas9 endonuclease coupled with a gRNA designed against the genomic sequence of interest (a CRISPR-Cas9 system), or a Cms1 endonuclease coupled with a gRNA designed against the genomic sequence of interest (a CRISPR-Cms1) can be used. Other nuclease systems for use with the methods of the present invention include CRISPR systems (e.g., Type I, Type II, Type III, Type IV, and/or Type V CRISPR systems (Makarova et al 2020 Nat Rev Microbiol 18:67-83)) with their corresponding gRNA(s), TALENs, zinc finger nucleases (ZFNs), meganucleases, and the like. Alternatively, a deactivated CRISPR nuclease (e.g., a deactivated Cas9, Cas12a, or Cms1 endonuclease) fused to a transcriptional regulatory element can be targeted to the upstream regulatory region of bZIP transcription factor gene, thereby modulating the function of the bZIP SIRT element (Piatek et al. (2015) Plant Biotechnol J 13:578-589).

The nuclease (or encoding nucleic acid), the guide RNA(s) (or encoding DNA), and the optional donor polynucleotide(s) of the present disclosure can be introduced into a plant cell, organelle, or plant embryo by a variety of means of transformation, including microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J 3:2717-2722), and ballistic particle acceleration [see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens)]; all of which are herein incorporated by reference. Site-specific genome editing of plant cells by biolistic introduction of a ribonucleoprotein comprising a nuclease and suitable guide RNA has been demonstrated (Svitashev et al (2016) Nat Commun doi: 10.1038/ncomms13274); these methods are herein incorporated by reference.

Agrobacterium- and biolistic-mediated transformation remain the two predominantly employed approaches. However, transformation may be performed by infection, transfection, microinjection, electroporation, microprojection, biolistics or particle bombardment, electroporation, silica/carbon fibers, ultrasound mediated, PEG mediated, calcium phosphate co-precipitation, polycation DMSO technique, DEAE dextran procedure, Agrobacterium and viral mediated (Caulimoriviruses, Geminiviruses, RNA plant viruses), liposome mediated and the like.

The nuclease polypeptides (or encoding nucleic acid), the guide RNA(s) (or DNAs encoding the guide RNA), and the optional donor polynucleotide(s) can be introduced into the plant cell, organelle, or plant embryo simultaneously or sequentially. The ratio of the nuclease (or encoding nucleic acid) to the guide RNA(s) (or encoding DNA) generally will be about stoichiometric such that the two components can form an RNA-protein complex with the target DNA. In one embodiment, DNA encoding a nuclease and DNA encoding a guide RNA are delivered together within the plasmid vector.

Alteration of the bZIP SIRT element function may also be achieved through the use of transposable element technologies to alter gene expression. It is well understood that transposable elements can alter the expression of nearby DNA (McGinnis et al. (1983) Cell 34:75-84). Alteration of the bZIP SIRT element function may be achieved by inserting a transposable element upstream of the bZIP transcription factor gene (e.g., a bZIP SIRT element).

Alteration of gene encoding a SIRT (e.g., SIRT) protein expression may also be achieved through expression of a transcription factor or transcription factors that regulate the expression of the gene encoding SIRT. Alteration of gene encoding a SIRT protein expression may be achieved by altering the expression of transcription factor(s) that are known to interact with a gene encoding a SIRT protein of interest.

The cells that have been transformed may be grown into plants (i.e., cultured) in accordance with conventional ways. See, for example, McCormick et al. (1986) Plant Cell Reports 5:81-84. In this manner, the present invention provides transformed plants or plant parts, transformed seed (also referred to as “transgenic seed”) or transformed plant progenies having a nucleic acid modification stably incorporated into their genome.

While the present disclosure is described in terms of transformed plants, it is recognized that transformed organisms of the invention also include plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the disclosure, provided that these parts comprise the introduced mutations or polynucleotides.

The present invention may be used for transformation of any plant species, e.g., both monocots and dicots. A plant or plant part to be transformed according to the methods pf the present disclosure can be a crop plant or part of a crop plant. Examples of crop plants include, but are not limited to, tomato (Solanum lycopersicum), eggplant (Solanum melongena), pepper (Capsicum annuum), corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana), sunflower (Helianthus annuus), quinoa (Chenopodium quinoa), chicory (Cichorium intybus), lettuce (Lactuca sativa), safflower (Carthamus tinctorius), wheat (Triticum aestivum), soybean (Glycine max), tobacco (Nicotiana spp., e.g., Nicotiana tabacum, Nicotiana sylvestris), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), grapes (Vitis vinifera, Vitis riparia), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), pea (Pisum sativum), eucalyptus (Eucalyptus spp.), oats (Avena sativa), barley (Hordeum vulgare), vegetables, ornamentals, and conifers. Additionally, a plant or plant part of the present disclosure can be an oilseed plant (e.g., canola (Brassica napus), cotton (Gossypium sp.), camelina (Camelina sativa) and sunflower (Helianthus sp.)), or other species including wheat (Triticum sp., such as Triticum aestivum L. ssp. aestivum (common or bread wheat), other subspecies of Triticum aestivum, Triticum turgidum L. ssp. durum (durum wheat, also known as macaroni or hard wheat), Triticum monococcum L. ssp. monococcum (cultivated einkom or small spelt), Triticum timopheevi ssp. timopheevi, Triticum turgigum L. ssp. dicoccon (cultivated emmer), and other subspecies of Triticum turgidum (Feldman)), barley (Hordeum vulgare), maize (Zea mays), oats (Avena sativa), or hemp (Cannabis sativa). Additionally, a plant or plant part of the present disclosure can be a forage plant or part of a forage plant. Examples of forage plants include legumes and crop plants described herein as well as grass forages including Agrostis spp., Lolium spp., Festuca spp., Poa spp., and Bromus spp. A plant or plant part of the present disclosure can be a legume, i.e., a plant belonging to the family Fabaceae (or Leguminosae), or a part (e.g., fruit or seed) of such a plant. Examples of legume include, without limitation, soybean (Glycine max), beans (Phaseolus spp., Vigna spp.), common bean (Phaseolus vulgaris), mung bean (Vigna radiata), cowpea (Vigna unguiculata), adzuki bean (Vigna angularis), fava bean (Vicia faba), pea (Pisum sativum), chickpea (Cicer arietinum), peanut (Arachis hypogaea), lentils (Lens culinaris, Lens esculenta), lupins (Lupinus spp.), white lupin (Lupinus albus), mesquite (Prosopis spp.), carob (Ceratonia siliqua), tamarind (Tamarindus indica), alfalfa (Medicago sativa), barrel medic (Medicago truncatula), birdsfood trefoil (Lotus japonicus), licorice (Glycyrrhiza glabra), and clover (Trifolium spp.). For example, a plant or plant part of the present disclosure can be Glycine max or Pisum sativum.

Compositions of the systems and constructs that can be used in the methods disclosed herein for introducing one or more mutations (e.g., insertions, substitutions, or deletions) to the upstream regulatory region of bZIP (e.g., bZIP SIRT element) is further disclosed in detail below.

C. Systems and Constructs for Mutating bZIP Upstream Regulatory Region

Described herein are systems and expression constructs that can be used for introducing one or more mutations (e.g., insertions, substitutions, or deletions) to the bZIP upstream regulatory region (e.g., bZIP SIRT element) in the genome of a plant cell or plant part, according to methods described in the present disclosure.

### 1. Nuclease

Nucleases that can be used in precise genome-editing technologies to modulate the expression of the endogenous sequence include, but are not limited to, meganucleases designed against the plant genomic sequence of interest (D'Halluin et al (2013) Plant Biotechnol J 11: 933-941); Cas9 endonuclease; Cas12a (Cpf1) endonuclease; Cms1 endonuclease; transcription activator-like effector nucleases (TALENs); zinc finger nucleases (ZFNs); a deactivated CRISPR nuclease (e.g., a deactivated Cas9, Cas12a, or Cms1 endonuclease) fused to a transcriptional regulatory element (Piatek et al. (2015) Plant Biotechnol J 13:578-589).

In some embodiments, the nuclease of the present invention is a CRISPR nuclease. In some embodiments, the CRISPR nuclease is a Cas12a nuclease, herein used interchangeably with a Cpf1 nuclease, e.g., a McCpf1 nuclease, e.g., Gen6 McCpf1.2_2c nuclease. In some embodiments, the CRISPR nuclease shares at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with SEQ ID NO: 20.

### 2. Guide RNA

To introduce one or more mutations into the upstream regulatory region of the bZIP transcription factor gene (e.g., the bZIP SIRT element), antisense constructions, complementary to at least a portion of the messenger RNA (mRNA) for the sequences of the upstream regulatory region of the bZIP transcription factor gene (e.g., the bZIP SIRT element) can be constructed. Antisense nucleotides are designed to hybridize with the corresponding mRNA. Modifications of the antisense sequences may be made as long as the sequences hybridize to and interfere with expression of the corresponding mRNA. In this manner, antisense constructions having at least 75%, optimally 80%, more optimally 85%, 90%, 95% or greater sequence identity to the corresponding sequences to be edited may be used. Furthermore, portions of the antisense nucleotides may be used to disrupt the expression of the target gene.

Accordingly, a recombinant DNA construct of the present disclosure may contain a guide RNA (gRNA) cassette to drive genome editing at the SIRT gene locus. For example, a recombinant DNA construct of the present disclosure may contain a gRNA cassette to drive a deletion (e.g., 7-9 nucleotide deletion) within the bZIP SIRT element. The gRNA can be specific to the bZIP SIRT element. For example, the gRNA can be specific to a nucleic acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence of SEQ ID NO: 3 or 4. In particular instances, the gRNA can drive a deletion within the bZIP SIRT element of Solanum lycopersicum (e.g., SlbZIP1 or SlbZIP2), and is specific to the nucleic acid sequence of SEQ ID NOs: 3 or 4. In some instances, a gRNA may comprise a targeting region that is complementary to a targeted sequence as well as another region that allows the gRNA to form a complex with a nuclease (e.g., a CRISPR nuclease) of interest. The targeting region of a gRNA for use in the method described herein may be 10-40 nucleotides long (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides long). For example, the targeting region of a gRNA for use in the method described herein may be 24 nucleotides in length. In some embodiments, the targeting region of a gRNA is encoded by a nucleic acid sequence comprising a nucleic acid sequence having at least 75% % (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence of any one of SEQ ID NOs: 12-19. In particular instances, the targeting region of a gRNA for use in the method described herein is encoded by a nucleic acid sequence comprising the nucleic acid sequence of any one of SEQ ID NOs: 12-19. In some embodiments, a gene editing efficiency of the one or more gRNAs is greater than 2% (e.g., 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 100%).

### 3. Promoter

As used herein, “promoter” refers to a regulatory region of DNA that is capable of driving expression of a sequence in a plant or plant cell. A number of promoters may be used in the practice of the disclosure. The promoter may have a constitutive expression profile. Constitutive promoters include the CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J 3:2723-2730); ALS promoter (U.S. Pat. No. 5,659,026), and the like.

Alternatively, promoters for use in the methods of the present disclosure can be tissue-preferred promoters. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Leaf-preferred promoters are also known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

Alternatively, promoters for use in the methods of the present disclosure can be developmentally-regulated promoters. Such promoters may show a peak in expression at a particular developmental stage. Such promoters have been described in the art, e.g., U.S. Pat. No. 10,407,670; Gan and Amasino (1995) Science 270: 1986-1988; Rinehart et al. (1996) Plant Physiol 112: 1331-1341; Gray-Mitsumune et al. (1999) Plant Mol Biol 39: 657-669; Beaudoin and Rothstein (1997) Plant Mol Biol 33: 835-846; Genschik et al. (1994) Gene 148: 195-202, and the like.

Alternatively, promoters for use in the methods of the present disclosure can be promoters that are induced following the application of a particular biotic and/or abiotic stress. Such promoters have been described in the art, e.g., Yi et al. (2010) Planta 232: 743-754; Yamaguchi-Shinozaki and Shinozaki (1993) Mol Gen Genet 236: 331-340; U.S. Pat. No. 7,674,952; Rerksiri et al. (2013) Sci World J 2013: Article ID 397401; Khurana et al. (2013) PLoS One 8: e54418; Tao et al. (2015) Plant Mol Biol Rep 33: 200-208, and the like.

Alternatively, promoters for use in the methods of the present disclosure can be cell-preferred promoters. Such promoters may preferentially drive the expression of a downstream gene in a particular cell type such as a mesophyll or a bundle sheath cell. Such cell-preferred promoters have been described in the art, e.g., Viret et al. (1994) Proc Natl Acad USA 91: 8577-8581; U.S. Pat. Nos. 8,455,718; 7,642,347; Sattarzadeh et al. (2010) Plant Biotechnol J 8: 112-125; Engelmann et al. (2008) Plant Physiol 146: 1773-1785; Matsuoka et al. (1994) Plant J 6: 311-319, and the like.

It is recognized that a specific, non-constitutive expression profile may provide an improved plant phenotype relative to constitutive expression of a gene or genes of interest. For instance, many plant genes are regulated by light conditions, the application of particular stresses, the circadian cycle, or the stage of a plant's development. These expression profiles may be important for the function of the gene or gene product in planta. One strategy that may be used to provide a desired expression profile is the use of synthetic promoters containing cis-regulatory elements that drive the desired expression levels at the desired time and place in the plant. Cis-regulatory elements that can be used to alter gene expression in planta have been described in the scientific literature (Vandepoele et al. (2009) Plant Physiol 150: 535-546; Rushton et al. (2002) Plant Cell 14: 749-762). Cis-regulatory elements may also be used to alter promoter expression profiles, as described in Venter (2007) Trends Plant Sci 12: 118-124.

### 4. Transfer DNA

A recombinant DNA construct described herein may contain transfer DNA (T-DNA) sequences. For example, a recombinant DNA construct of the present disclosure may contain T-DNA of tumor-inducing (Ti) plasmid of Agrobacterium tumefaciens. Alternatively, a recombinant DNA construct of the present disclosure may contain T-DNA of tumor-inducing (Ti) plasmid of Agrobacterium rhizogenes. The vir genes of the Ti plasmid may help in transfer of T-DNA of a recombinant DNA construct into nuclear DNA genome of a host plant. For example, Ti plasmid of Agrobacterium tumefaciens may help in transfer of T-DNA of a recombinant DNA construct of the present disclosure into nuclear DNA genome of a host plant, thus enabling the transfer of a gRNA of the present disclosure into nuclear DNA genome of a host plant (e.g., a pea plant).

Also described herein is a bacterium containing a recombinant DNA construct of the present disclosure. For example, the present disclosure may provide an Agrobacterium tumefaciens containing a recombinant DNA construct that comprises a gRNA to drive genome editing at the SlbZIP SIRT gene locus.

### 5. Regulatory Signals

In some embodiments, a recombinant DNA construct described herein may contain additional regulatory signals, including, but not limited to, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EPO 0480762A2; Sambrook et al. (1992) Molecular Cloning: A Laboratory Manual, ed. Maniatis et al. (Cold Spring Harbor Laboratory Press, Cold Spring Harbor, N.Y.), hereinafter “Sambrook 11”; Davis et al., eds. (1980) Advanced Bacterial Genetics (Cold Spring Harbor Laboratory Press), Cold Spring Harbor, N.Y., and the references cited therein.

### 6. Reporter Genes Selectable Marker Genes

Reporter genes or selectable marker genes may also be included in the expression cassettes of the present invention. Examples of suitable reporter genes known in the art can be found in, for example, Jefferson, et al., (1991) in Plant Molecular Biology Manual, ed. Gelvin, et al., (Kluwer Academic Publishers), pp. 1-33; DeWet, et al., (1987) Mol. Cell. Biol. 7:725-737; Goff, et al., (1990) EMBO J. 9:2517-2522; Kain, et al., (1995) Bio Techniques 19:650-655 and Chiu, et al., (1996) Current Biology 6:325-330, herein incorporated by reference in their entirety.

Selectable marker genes for selection of transformed cells or tissues can include genes that confer antibiotic resistance or resistance to herbicides. Examples of suitable selectable marker genes include, but are not limited to, genes encoding resistance to chloramphenicol (Herrera Estrella, et al., (1983) EMBO J 2:987-992); methotrexate (Herrera Estrella, et al., (1983) Nature 303:209-213; Meijer, et al., (1991) Plant Mol. Biol. 16:807-820); hygromycin (Waldron, et al., (1985) Plant Mol. Biol. 5:103-108 and Zhijian, et al., (1995) Plant Science 108:219-227); streptomycin (Jones, et al., (1987) Mol. Gen. Genet. 210:86-91); spectinomycin (Bretagne-Sagnard, et al., (1996) Transgenic Res. 5:131-137); bleomycin (Hille, et al., (1990) Plant Mol. Biol. 7:171-176); sulfonamide (Guerineau, et al., (1990) Plant Mol. Biol. 15:127-36); bromoxynil (Stalker, et al., (1988) Science 242:419-423); glyphosate (Shaw, et al., (1986) Science 233:478-481 and U.S. patent application Ser. Nos. 10/004,357 and 10/427,692); phosphinothricin (DeBlock, et al., (1987) EMBO J 6:2513-2518), herein incorporated by reference in their entirety.

Other selectable marker genes that could be employed on the expression constructs disclosed herein include, but are not limited to, GUS (beta-glucuronidase; Jefferson, (1987) Plant Mol. Biol. Rep. 5:387), GFP (green fluorescence protein; Chalfie, et al., (1994) Science 263:802), luciferase (Riggs, et al., (1987) Nucleic Acids Res. 15(19):8115 and Luehrsen, et al., (1992) Methods Enzymol. 216:397-414) and the maize genes encoding for anthocyanin production (Ludwig, et al., (1990) Science 247:449), herein incorporated by reference in their entirety.

### 7. Terminators

A transcription terminator may also be included in the expression cassettes of the present invention. Plant terminators are known in the art and include those available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

### 8. Vectors

Also disclosed herein are vectors containing recombinant DNA constructs. As used herein, “vector” refers to a nucleotide molecule (e.g., a plasmid, cosmid), bacterial phage, or virus for introducing a nucleotide construct, for example, a recombinant DNA construct, into a host cell. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

In some embodiments, provided herein are expression cassettes located on a vector comprising gRNA sequence specific for bZIP SIRT element gene.

In some embodiments, a vector is a plasmid containing a recombinant DNA construct of the present disclosure. For example, the present disclosure may provide a plasmid containing a recombinant DNA construct that comprises a gRNA to drive genome editing at the bZIP SIRT gene locus.

In some embodiments, a vector is a recombinant virus containing a recombinant DNA construct of the present disclosure. For example, the present disclosure may provide a recombinant virus containing a recombinant DNA construct that comprises a gRNA, wherein the gRNA can drive genome editing at the bZIP SIRT element gene locus. A recombinant virus described herein can be a recombinant lentivirus, a recombinant retrovirus, a recombinant cucumber mosaic virus (CMV), a recombinant tobacco mosaic virus (TMV), a recombinant cauliflower mosaic virus (CaMV), a recombinant odontoglossum ringspot virus (ORSV), a recombinant tomato mosaic virus (ToMV), a recombinant bamboo mosaic virus (BaMV), a recombinant cowpea mosaic virus (CPMV), a recombinant potato virus X (PVX), a recombinant Bean yellow dwarf virus (BeYDV), or a recombinant turnip vein-clearing virus (TVCV).

D. Construct Comprising Mutated bZIP Upstream Regulatory Region

The coding sequences of the present invention, when assembled within a DNA construct such that a promoter is operably linked to the coding sequence of interest, enable expression and accumulation of bZIP comprising a mutated upstream regulatory region (e.g., SlbZIP1 SIRT) in the cells of a plant stably transformed with this DNA construct.

In this manner, the nucleic acid sequences encoding a mutated upstream regulatory region of bZIP (e.g., SlbZIP1 SIRT) of the invention are provided in expression cassettes or expression constructs along with a promoter sequence of interest, typically a heterologous promoter sequence, for expression in the plant of interest. By “heterologous promoter sequence” is intended to mean a sequence that is not naturally operably linked with the bZIP-encoding nucleic acid sequence. For instance, a 2×35s promoter or a native promoter may be operably linked to the nucleic acid sequences comprising mutated upstream regulatory region of bZIP. Either the bZIP-encoding nucleic acid sequence or the heterologous promoter sequence may be homologous, or native, or heterologous, or foreign, to the plant host. It is recognized that the promoter may also drive expression of its homologous or native nucleic acid sequence. In this case, the transformed plant will have a change in phenotype.

Accordingly, in some aspects, the present disclosure provides DNA constructs comprising, in operable linkage:
a. a promoter that is functional in a plant cell; and
b. a nucleic acid sequence encoding a bZIP upstream regulatory region comprising the SIRT element with one or more mutations that alter expression or function of the bZIP SIRT;
wherein introducing into a plurality of cells in a plant with the DNA construct results in alteration of bZIP expression or function, and/or generation of a plant with increased sugar and/or TSS content.

In some embodiments, the DNA construct further comprises, in operable linkage, a bZIP transcription factor gene linked downstream of the SIRT element. In some embodiments, the DNA construct further comprises, in operable linkage, a reporter construct (e.g., GFP, a HA tag).

In some embodiments, the DNA construct comprises, in operable linkage, a 2×35s promoter, a mutated bZIP SIRT element, GFP, and a NOSt terminator. In some embodiments, the DNA construct comprises, in operable linkage, a native promoter, a mutated bZIP SIRT element, bZIP, a HA tag, and a terminator.

The upstream regulatory region of bZIP (e.g., bZIP SIRT) may comprise any mutation(s) (e.g., one or more insertions, substitutions or deletions) described in the present disclosure, as compared to a wild-type sequence. For instance, in some embodiments, the mutated nucleic acid sequence may comprise one or more insertions, substitutions or deletions of about 1-84, 2-84, 3-84, 4-84, 5-84, 6-84, 7-84, 8-84, 9-84, 10-84, or 11-84 (e.g., about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, or 84) nucleotides of bZIP 5′UTR, bZIP uORF, or bZIP SIRT element in the genome of a plant cell or plant part. In particular, the mutated nucleic acid sequence may comprise one or more insertions, substitutions or deletions of about 7-9 nucleotides in the bZIP 5′UTR, bZIP uORF, or bZIP SIRT element. The mutation (e.g., insertion, substitution, or deletion) may comprise an in-frame mutation or an out-of-frame mutation of the bZIP SIRT element.

The one or more insertions, substitutions or deletions in the bZIP upstream regulatory region (e.g., the bZIP SIRT element) may be in a nucleotide region that comprises a nucleic acid sequence having at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence of SEQ ID NO: 3 or 4.

For example, the one or more insertions, substitutions or deletions in in the bZIP upstream regulatory region (e.g., bZIP SIRT element) may comprise a deletion of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, or 84 nucleotides in a nucleotide region that comprises the nucleic acid sequence of SEQ ID NO: 3 or 4. In particular, the one or more insertions, substitutions or deletions in the bZIP upstream regulatory region (e.g., bZIP SIRT element) may comprise a deletion of about 7-9 nucleotides. In some embodiments, the deletion is a deletion of nucleotide 6 through 13 of SEQ ID NO: 3, resulting in the sequence set forth in SEQ ID: 7; a deletion of nucleotide 53 through 60 of SEQ ID NO: 3, resulting in the sequence set forth in SEQ ID: 8; a deletion of nucleotide 53 through 61 of SEQ ID NO: 3, resulting in a sequence set forth in SEQ ID: 9; or a deletion of nucleotide 261 through 268 of SEQ ID NO: 1, resulting in a deletion of nucleotide 69 through 75 of SEQ ID NO: 3 and a sequence set forth in SEQ ID: 10. In some embodiments, the mutated SIRT element shares at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) identity with a sequence selected from the group consisting of SEQ ID No: 7-10.

In some embodiments, the one or more insertions, substitutions or deletions in the bZIP upstream region (e.g., bZIP SIRT) nucleic acid sequence may produce one or more amino acid changes (e.g., 1, 2, 3, 4, 5, 6, 7, 8, 9, 10, 11, 12, 13, 14, 15, 16, 17, 18, 19, 20, 21, 22, 23, 24, 25, 26, 27, or 28 amino acid changes) in the SIRT amino acid sequence that comprises the amino acid sequence of SEQ ID NO: 5 or 6 in the plant or plant part. In particular, the one or more insertions, substitutions or deletions in the bZIP upstream regulatory region (e.g., the bZIP SIRT element) may comprise 1, 2, or 3 amino acid changes in a SIRT peptide sequence. In some embodiments, the change comprises one or more of M1L, M4L, and M10L compared to the amino acid sequence of SEQ ID NO: 5. In some embodiments, the mutated SIRT element shares at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) identity with the amino acid sequence of SEQ ID NO: 11.

Provided herein are DNA constructs comprising the modified bZIP upstream regulatory regions (e.g., bZIP SIRT element); vectors comprising the modified bZIP upstream regulatory regions (e.g., bZIP SIRT element) or the DNA construct comprising the modified bZIP upstream regulatory regions (e.g., bZIP SIRT element); and cells comprising the modified bZIP upstream regulatory regions (e.g., bZIP SIRT element), the DNA construct comprising the modified bZIP upstream regulatory regions (e.g., bZIP SIRT element), or the vector comprising the modified bZIP upstream regulatory regions (e.g., bZIP SIRT element) according to the present disclosure.

In some aspects, the present disclosure provides methods for generating a plant with increased sugar and/or TSS content comprising introducing into the plant the DNA construct of the present disclosure. In some embodiments, the DNA construct is introduced into the plant by stable transformation. In other embodiments, the DNA construct is introduced into the plant by transient transformation. In some aspects, the present disclosure provides plants, plant parts (juice, pulp, seed, fruit, flowers, nectar, embryos, pollen, ovules, leaves, stems, branches, bark, kernels, ears, cobs, husks, stalks, roots, root tips, anthers, etc.), or plant products (e.g., plant extract, plant concentrate, plant powder, plant protein, and plant biomass) generated by comprising introducing into the plant the DNA construct of the present disclosure.

### V. Breeding of Plants

Also disclosed herein are methods for breeding a plant, such as a tomato, eggplant, or pepper plant that has been modified to contain one or more mutations in the bZIP SIRT upstream regulatory region, or to contain a polynucleotide containing, e.g., a gRNA specific to a bZIP SIRT element or a polynucleotide comprising an edited bZIP SIRT element (e.g., SEQ ID NOs: 7-11). A plant containing the one or more mutations or the polynucleotide of the present disclosure may be regenerated from a plant cell or plant part, wherein the genome of the plant cell or plant part is genetically-modified to contain the one or more mutations or the polynucleotide of the present disclosure. Using conventional breeding techniques or self-pollination, one or more seeds may be produced from the plant that contains the one or more mutations or the polynucleotide of the present disclosure. Such a seed, and the resulting progeny plant grown from such a seed, may contain the one or more mutations or the polynucleotide of the present disclosure, and therefore may be transgenic. Progeny plants are plants having a genetic modification to contain the one or more mutations or the polynucleotide of the present disclosure, which descended from the original plant having modification to contain the one or more mutations or the polynucleotide of the present disclosure. Seeds produced using such a plant of the invention can be harvested and used to grow generations of plants having genetic modification to contain the one or more mutations or the polynucleotide of the present disclosure, e.g., progeny plants, of the invention, comprising the polynucleotide and optionally expressing a gene of agronomic interest (e.g., herbicide resistance gene). Descriptions of breeding methods that are commonly used for different crops can be found in one of several reference books, see, e.g., Allard, Principles of Plant Breeding, John Wiley & Sons, NY, U. of CA, Davis, Calif., 50-98 (1960); Simmonds, Principles of Crop Improvement, Longman, Inc., NY, 369-399 (1979); Sneep and Hendriksen, Plant breeding Perspectives, Wageningen (ed), Center for Agricultural Publishing and Documentation (1979); Fehr, Soybeans: Improvement, Production and Uses, 2nd Edition, Monograph, 16:249 (1987); Fehr, Principles of Variety Development, Theory and Technique, (Vol. 1) and Crop Species Soybean (Vol. 2), Iowa State Univ., Macmillan Pub. Co., NY, 360-376 (1987).

### VI. Method of Selecting a Plant with Increased Sugar Content

In some aspects, the present disclosure provides methods of selecting a plant with increased sugar content, by introducing into a plant cell a gene editing system, wherein the gene editing system targets a sucrose induced repression of translation (SIRT) element of an upstream open reading frame (uORF) of a basic region/leucine zipper motif (bZIP) transcription factor gene or homologs thereof to generate at least one mutated SIRT elements, making DNA constructs each comprising, in operable linkage: a. a promoter that is functional in a plant cell; and b. a nucleic acid sequence encoding one of the mutated SIRT elements; transforming plants with the DNA constructs; evaluating a function of the modified SIRT elements in the transformed plants; and selecting a plant with increased sugar content, wherein the evaluating step comprises: quantifying an expression of a reporter gene linked to the modified SIRT element; quantifying, in the absence and the presence of sucrose, expression of one or more genes regulated by the bZIP transcription factor gene; and/or quantifying one or more of total sugar, TSS, sucrose, glucose, fructose, galactose, maltose, and lactose levels. In some embodiments, the one or more genes regulated by the bZIP transcription factor gene comprise one or more of asparagine synthase (ASN), proline dehydrogenase (PDH), sucrose phosphate synthase (SPS), sucrose-6′-phosphate phosphatase (SPP), and calmodulin.

It will be readily apparent to those skilled in the art that other suitable modifications and adaptations of the methods of the invention described herein are obvious and may be made using suitable equivalents without departing from the scope of the invention or the embodiments disclosed herein. Having now described the invention in detail, the same will be more clearly understood by reference to the following examples, which are included for purposes of illustration only and are not intended to be limiting. Unless otherwise noted, all parts and percentages are by dry weight.

### EXAMPLES

### Example 1: Gene Editing System Targeting SIRT Element

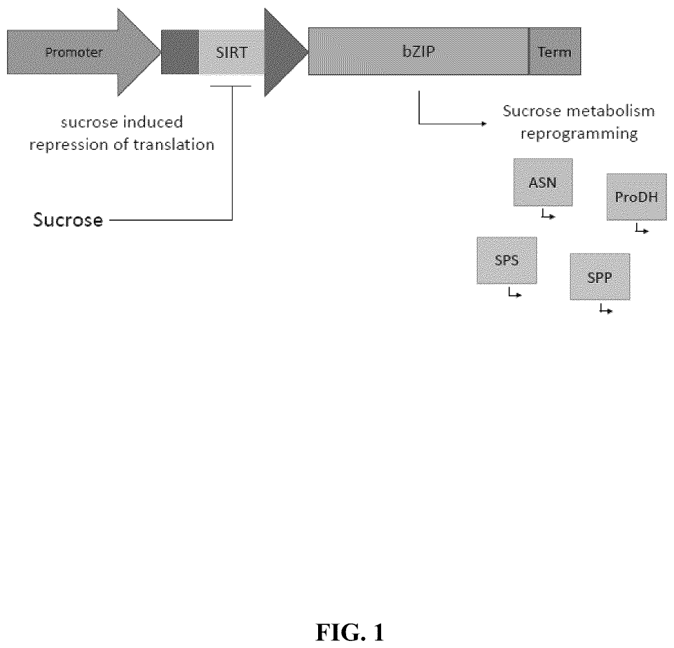

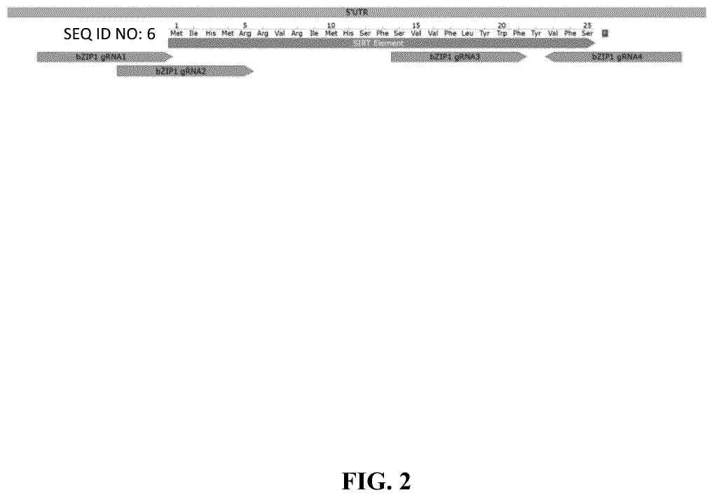

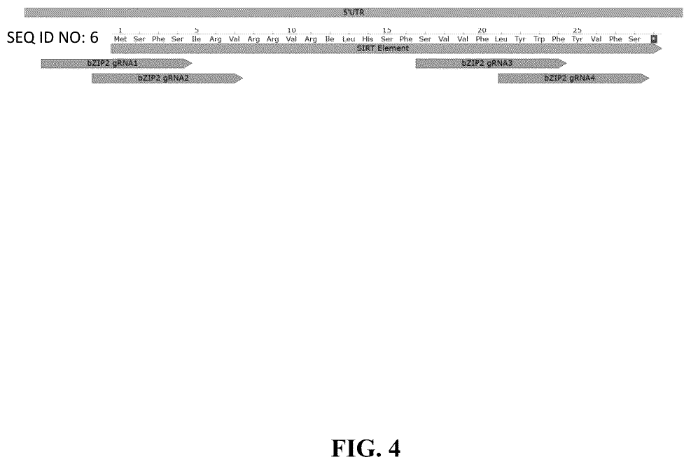

As depicted in FIG. 1, The SIRT element in the upstream regulatory region of the bZIP transcription factor represses bZIP expression or function in the presence of sucrose, thereby regulating expression and/or function of genes downstream of bZIP that are involved in sucrose metabolism, including asparagine synthase (ASN), proline dehydrogenase (PDH), sucrose phosphate synthase (SPS), and sucrose-6′-phosphate phosphatase (SPP). Alteration of the bZIP SIRT function may results in sucrose metabolism reprogramming, and thereby increased sugar or TSS content, in a plant or plant part. Accordingly, gRNAs targeting the bZIP1 SIRT element and the bZIP2 element were prepared, as schematically depicted in FIGS. 2 and 4, respectively. Four exemplary guide RNAs (gRNAs) targeting the bZIP1 SIRT element (bZIP1 gRNA1-4) are depicted in FIG. 2, along with the 5′UTR of the bZIP1 gene, including SlbZIP1 SIRT element. The targeting region of the four bZIP1 gRNA sequences are set forth as SEQ ID NO: 12-15. The nucleic acid sequence of SlbZIP1 SIRT element is set forth as SEQ ID NO: 3. The amino acid sequence of SlbZIP1 SIRT element is set forth as SEQ ID NO: 5. Four exemplary guide RNAs (gRNAs) targeting the bZIP2 SIRT element (bZIP1 gRNA1-4) are depicted in FIG. 4, along with the 5′UTR of the bZIP2 gene, including SlbZIP2 SIRT element. The targeting region of the four bZIP2 gRNA sequences are set forth as SEQ ID NO: 16-19. The nucleic acid sequence of SlbZIP1 SIRT element is set forth as SEQ ID NO: 4. The amino acid sequence of SlbZIP1 SIRT element is set forth as SEQ ID NO: 6.

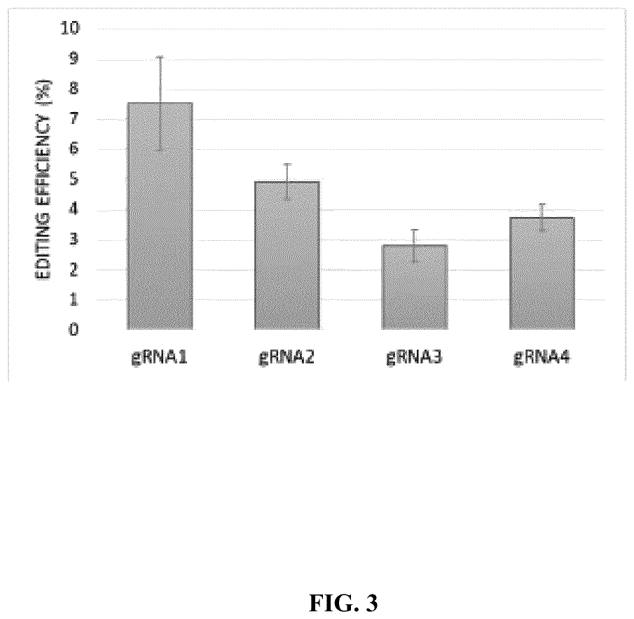

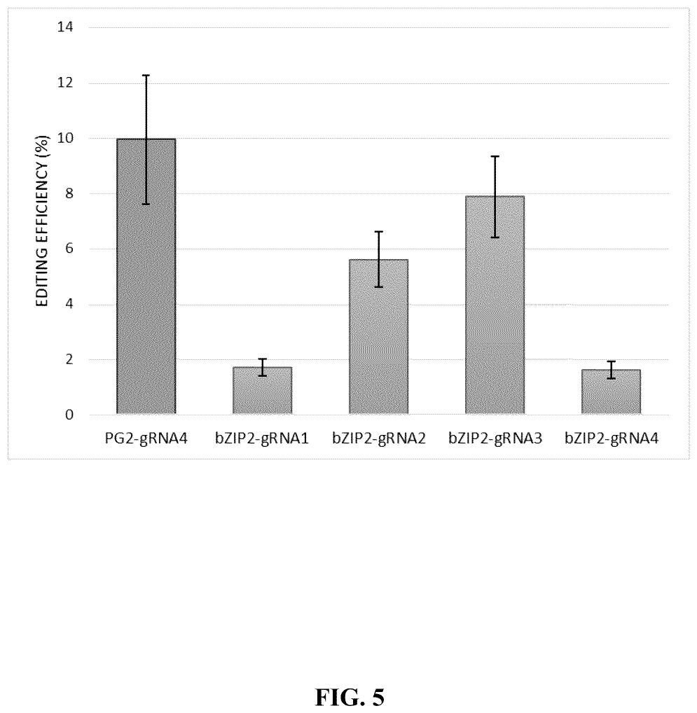

Tomato protoplasts were transformed with each gRNA cassette and Gen6 McCpf1 nuclease, and editing efficiency was quantified via next generation sequencing (NGS). As shown in FIG. 3, all of bZIP1 gRNA1-4 edited the bZIP1 SIRT element with Gen6 McCpf1. As shown in FIG. 5, all of bZIP2 gRNA1-4 edited the bZIP2 SIRT element with Gen6 McCpf1.

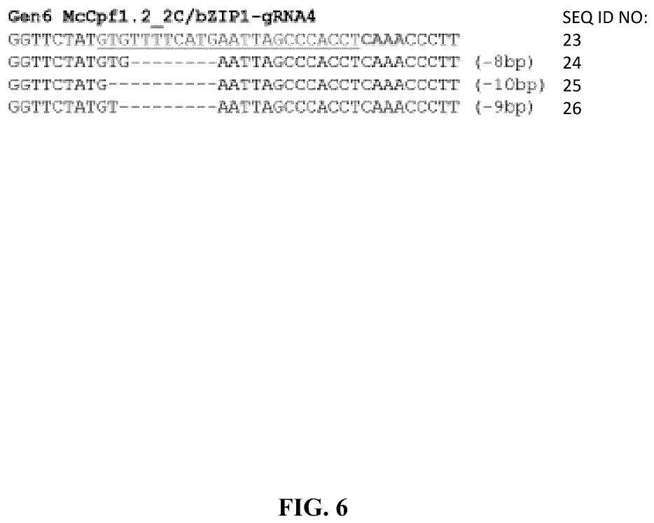

A number of SIRT mutants were generated using the gene editing system of the present disclosure. FIG. 6 depicts exemplary sequences of part of wild-type or mutant SlbZIP1 uORF including a SIRT element (SEQ ID NOs: 23-26). The first row represents the nucleic acid sequence of part of a wild-type SlbZIP1 uORF including the wild-type SlbZIP1 SIRT element (SEQ ID NO: 3). The second to the fourth rows depict the mutant sequences comprising a nucleotide deletion of 8 bp, 10 bp, and 9 bp, respectively, in the SlbZIP uORF as compared to the wild-type SlbZIP uORF. The mutations were introduced in tomato protoplasts using gRNA4 and a Gen6 McCPF1 nuclease.

To further identify functional gRNAs in the SIRT region, gRNA cassettes plus a nuclease [e.g., Cas12a (Cpf1) endonuclease, meganuclease, Cas9 endonuclease, Cms1 endonuclease; transcription activator-like effector nuclease (TALEN), zinc finger nuclease (ZFN)] are introduced into tomato protoplasts and editing efficiency is quantified via next generation sequencing (NGS). If editing efficiency in protoplasts is greater than 10%, the working gRNA/nuclease combination is cloned into a binary vector. Functional gRNAs targeting other bZIP SIRT homologs/orthologs are similarly prepared.

### Example 2: SIRT Modification Results in Increased Basal Expression and Decreased Regulation by Sucrose of Downstream Target

Modified SIRT sequences were tested for changes in basal expression and regulation by sucrose of downstream ORF.

### 1. SIRT-GFP Construct

Expression cassettes driven by the 2×35s promoter with 8 independent synthetic deletions in SIRT element (4659 Mut-SIRT-4666 Mut-SIRT) upstream of GFP were generated. The exemplary expression cassettes are depicted for instance in FIGS. 1 and 8-11. The cassettes with synthetic deletions, as well as no mutations (WT-SIRT) were transiently expressed in tobacco leaves. GFP protein levels in infiltrated leaves were quantified after dark treatment plus/minus sucrose using similar assays described in Thalor et al (2012), PLoS One, 7(3):33111, which is herein incorporated by reference in its entirety.

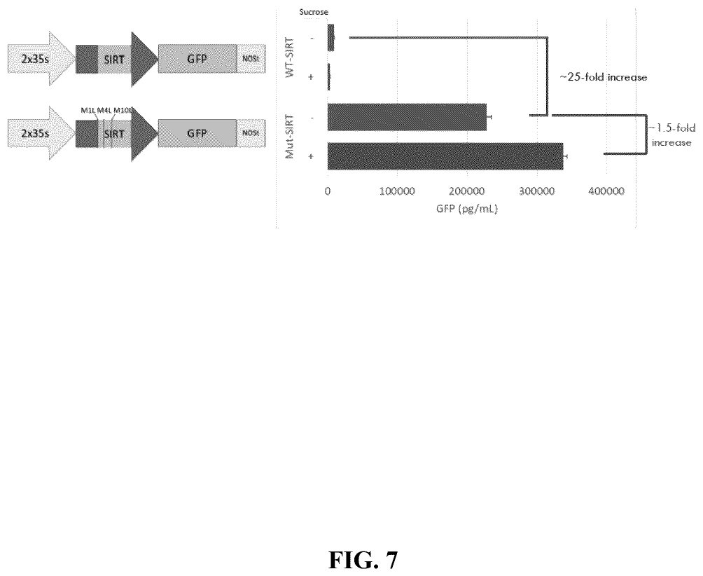

As shown in FIG. 7, plants infiltrated with Mut-SIRT (with M1L, M4L, and M10L amino acid substitutions, SEQ ID NO: 11) showed approximately 25-fold increase in basal expression levels of GFP in the absence of sucrose as compared to plants infiltrated with WT-SIRT (SEQ ID NO: 5). Further, plants infiltrated with WT-SIRT showed a decrease in GFP expression levels in the presence of sucrose as compared to baseline (i.e., in the absence of sucrose). On the other hand, plants infiltrated with Mut-SIRT showed approximately 1.5-fold increase in GFP expression levels in the presence of sucrose as compared to baseline (i.e., in the absence of sucrose).

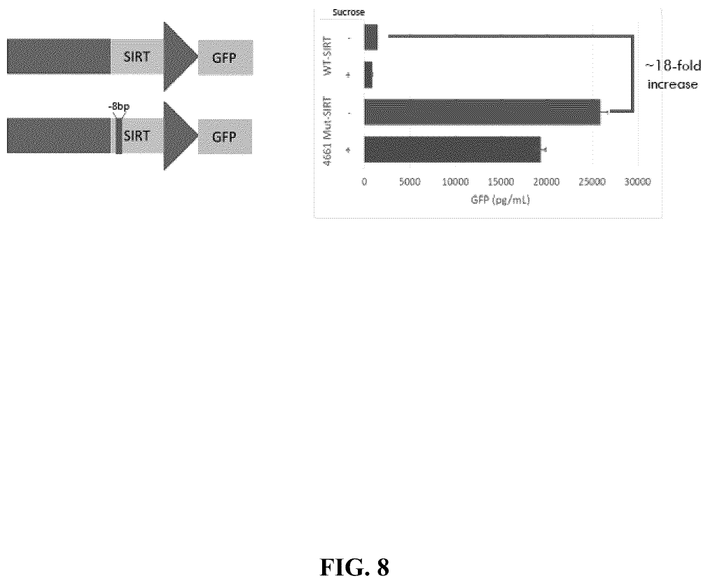

As shown in FIG. 8, plants infiltrated with 4661 Mut-SIRT (with an 8 bp deletion, SEQ ID NO: 7) showed approximately 18-fold increase in basal expression levels of GFP in the absence of sucrose as compared to plants infiltrated with WT-SIRT (SEQ ID NO: 3). Plants infiltrated with WT-SIRT and 4661 Mut-SIRT both showed a decrease in GFP expression levels in the presence of sucrose as compared to baseline (i.e., in the absence of sucrose). However, plants infiltrated with 4661 Mut-SIRT showed approximately 18-fold increase in GFP expression levels as compared to plants infiltrated with WT-SIRT in the presence of sucrose.

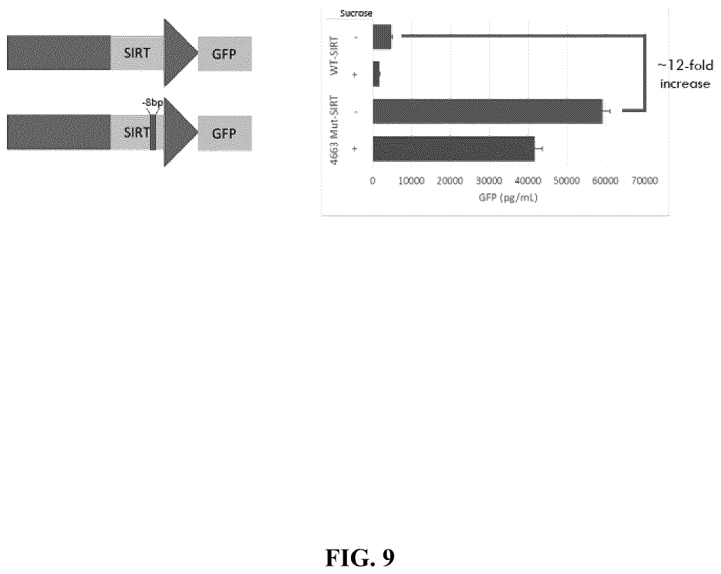

As shown in FIG. 9, plants infiltrated with 4663 Mut-SIRT (with an 8 bp deletion, SEQ ID NO: 8) showed approximately 12-fold increase in basal expression levels of GFP in the absence of sucrose as compared to plants infiltrated with WT-SIRT (SEQ ID NO: 3). Plants infiltrated with WT-SIRT and 4663 Mut-SIRT both showed a decrease in GFP expression levels in the presence of sucrose as compared to baseline (i.e., in the absence of sucrose). However, plants infiltrated with 4663 Mut-SIRT showed approximately 21-fold increase in GFP expression levels in the presence of sucrose as compared to plants infiltrated with WT-SIRT.

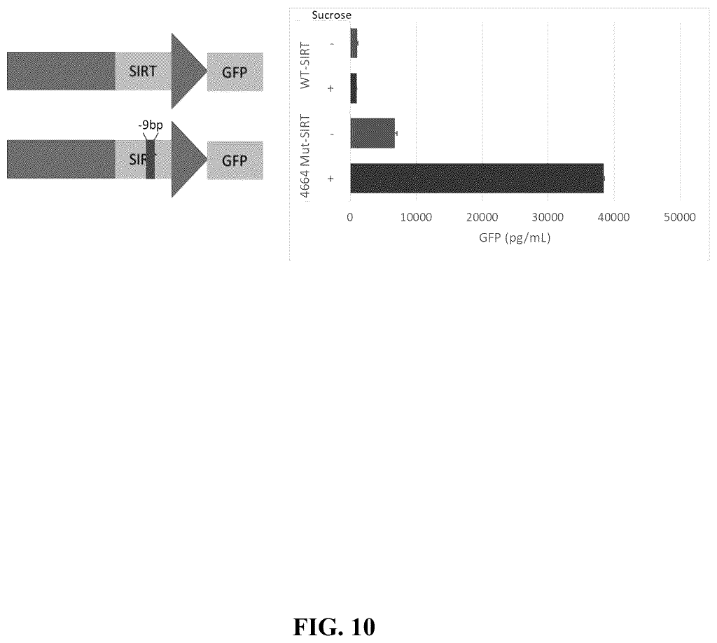

As shown in FIG. 10, plants infiltrated with 4664 Mut-SIRT (with an 8 bp deletion, SEQ ID NO: 9) showed approximately 6-fold increase in basal expression levels of GFP in the absence of sucrose as compared to plants infiltrated with WT-SIRT (SEQ ID NO: 3). Further, sucrose did not have inhibitory regulation in plants infiltrated with 4664 Mut-SIRT, which showed approximately 6-fold increase in GFP expression levels in the presence of sucrose as compared to baseline (i.e., in the absence of sucrose).

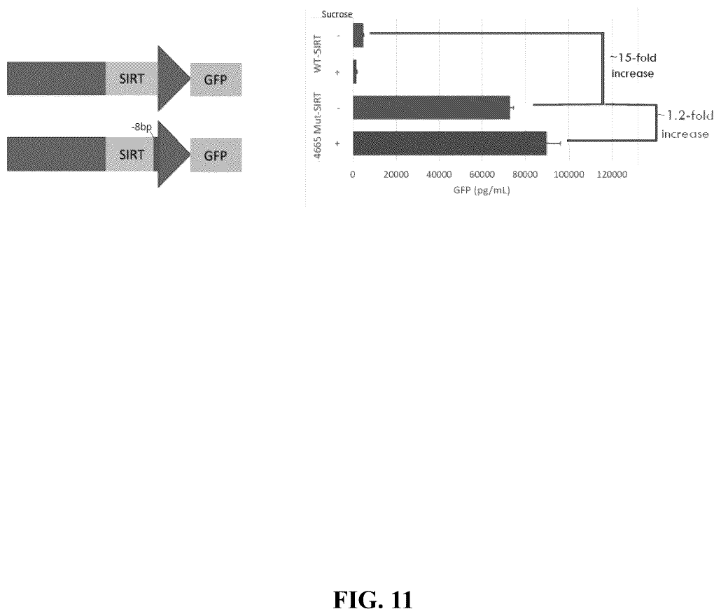

As shown in FIG. 11, plants infiltrated with 4665 Mut-SIRT (with an 8 bp deletion in SlbZIP1 uORF including a 7 bp deletion in the SlbZIP1 SIRT element, set forth as SEQ ID NO: 10) showed approximately 15-fold increase in basal expression levels of GFP in the absence of sucrose as compared to plants infiltrated with WT-SIRT (SEQ ID NO: 3). Further, sucrose did not have inhibitory regulation in plants infiltrated with 4665 Mut-SIRT, which showed approximately 1.2-fold increase in GFP expression levels in the presence of sucrose as compared to baseline (i.e., in the absence of sucrose).

Effects of the 8 independent synthetic deletions in the SlbZIP1 SIRT region on expression of the operably-linked downstream gene (GFP) in the absence and presence of sucrose is summarized in Table 1 below.

TABLE 1
GFP Expression in Tobacco Leaves Transiently Expressing
SIRT-GFP Construct with Various SIRT Mutations

| | Biological Replicate #1 | | Biological Replicate #2 | |
|---|---|---|---|---|
| | GFP Baseline | Inhibition by | GFP Baseline | Inhibition by |
| Construct | Expression | Sucrose | Expression | Sucrose |
| 4659 | ~3-fold | YES | N/A | N/A |
| Mut-SIRT | Increase | | | |
| 4660 | ~3-fold | YES | N/A | N/A |
| Mut-SIRT | Decrease | | | |
| 4661 | ~18-fold | YES | ~11-fold | YES |
| Mut-SIRT | Increase | | Increase | |
| 4662 | ~5-fold | YES | N/A | N/A |
| Mut-SIRT | Increase | | | |
| 4663 | ~12-fold | YES | ~12-fold | NO |
| Mut-SIRT | Increase | | Increase | |
| 4664 | ~6-fold | NO | ~3-fold | NO |
| Mut-SIRT | Increase | | Increase | |
| 4665 | ~15-fold | NO | ~13fold | NO |
| Mut-SIRT | Increase | | Increase | |
| 4666 | ~7-fold | NO | N/A | N/A |
| Mut-SIRT | Increase | | | |

These results demonstrate that mutations (e.g., insertions, substitutions, deletions) of the bZIP SIRT element are capable of increasing baseline expression levels of genes downstream of SIRT and/or removing sucrose induced repression of genes downstream of SIRT. Because SlbZIP regulates a number of genes involved in carbohydrate metabolism or transport, e.g., asparagine synthase (ASN), proline dehydrogenase (PDH), sucrose phosphate synthase (SPS), sucrose-6′-phosphate phosphatase (SPP), and calmodulin, introducing one or more mutations into a bZIP SIRT element can alter carbohydrate metabolism and/or transport, thereby increasing sugar or TSS content in tomato plants or plant parts.

### 2. SIRT-bZIP-HA Construct

Expression cassettes driven by native promoter with independent synthetic deletions in SIRT element upstream of HA-tagged bZIP were generated. The cassettes comprising SIRT with synthetic deletions, as well as no mutations (WT-SIRT) are transiently expressed in tobacco leaves using Agrobacterium. bZIP protein levels in infiltrated leaves are quantified after dark treatment plus/minus sucrose using similar assays as used in Thalor et al (2012).

### Example 3: SIRT Modification and Expression of Target Genes Downstream of bZIP in Tomato Plants

Mutant SIRT/bZIP constructs are tested in tomato plants to study whether the SIRT mutations that remove sucrose repression of bZIP or otherwise enhance expression or function of bZIP modify expression or function of targets downstream of bZIP. Three (3) to 4 week old tomato plants are infiltrated with modified SIRT constructs (expression cassettes driven by native promoter with independent synthetic deletions in SIRT element upstream of HA-tagged bZIP), described and validated in Example 2, to transiently express the bZIP/SIRT cassettes. At day 3 post-infiltration, sampling is conducted and plants are tested for mutations.

Levels of mRNA and/or protein expression of bZIP downstream targets, e.g., asparagine synthase (ASN), proline dehydrogenase (PDH), sucrose phosphate synthase (SPS), sucrose-6′-phosphate phosphatase (SPP), and calmodulin is tested in tomato leaves using methods well known in the art, to determine if sucrose metabolism reprogramming genes are affected by mutations introduced to the SIRT element.

### Example 4: SIRT Modification and Change in Total Soluble Solids (TSS) or Sugar Levels

Whether bZIP with mutated SIRT element results in increased TSS and/or sugar levels in ripe fruit is tested in tomato plants. Whether the SIRT mutations resulting in release of sucrose repression of bZIP and/or upregulation of bZIP expression or function positively impact TSS and/or sugar levels in tomato fruit is tested. bZIPT expression cassettes driven by native promoter with synthetic deletion, generated and validated in Example 2 is used. Transformation of tomato plants with the expression cassettes is initiated over one week and at least 10 transgenic plants are generated. TO plants are grown, and the number of copies of transgenes each plant carries is determined. From each transgenic plant, at least 3 fruits are harvested at full red stage. TSS, Total sugar, sucrose, glucose, fructose, galactose, maltose, and/or lactose levels are quantified in these fruits using methods well known in the art. Quantitative sugar assays with volatile and organic acid analysis are also performed in these fruits.

### Example 5: Generation of T0 Plants with Modified SIRT Upstream of bZIP

At least three heterozygous or homozygous SIRT mutant T0 tomato plants are generated in this Example. Transformation of tomato plants with working gene editing system (e.g., nuclease/gRNA combination) is initiated over two weeks. Early sampling is conducted at day 7 and 14 for NGS detection of mutations. At least 100 transgenic plants are generated. At least 3 SIRT mutant plants are identified using Sanger sequencing.

### Example 6: SIRT Modification and Change in Total Sugars

T0 plants are grown to generate homozygous SIRT loss of function plants. Sucrose, glucose, fructose, galactose, maltose, and lactose levels are compared in heterozygous and homozygous T1 progeny using methods well known in the art. Quantitative sugar assays with volatile and organic acid analysis are also conducted.

The section headings used herein are for organizational purposes only and are not to be construed as limiting the subject matter described in any way.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

While various aspects of the invention are described herein, it is not intended that the invention be limited by any particular aspect. On the contrary, the invention encompasses various alternatives, modifications, and equivalents, as will be appreciated by those of skill in the art. Furthermore, where feasible, any of the aspects disclosed herein may be combined with each other (e.g., the feature according to one aspect may be added to the features of another aspect or replace an equivalent feature of another aspect) or with features that are well known in the art, unless indicated otherwise by context.

TABLE 2
Sequence Description

| Sequence | |
|---|---|
| Identifier | Description |
| SEQ ID NO: 1 | Nucleic acid sequence for Solanum lycopersicum |
| | bZIP1 mRNA |
| SEQ ID NO: 2 | Nucleic acid sequence for Solanum lycopersicum |
| | bZIP2 mRNA |
| SEQ ID NO: 3 | Nucleic acid sequence for SIRT element of Solanum |
| | lycopersicum bZIP1 |
| SEQ ID NO: 4 | Nucleic acid sequence for SIRT element of Solanum |
| | lycopersicum bZIP2 |
| SEQ ID NO: 5 | Amino acid sequence for SIRT element of Solanum |
| | lycopersicum bZIP1 |
| SEQ ID NO: 6 | Amino acid sequence for SIRT element of Solanum |
| | lycopersicum bZIP2 |
| SEQ ID NO: 7 | Nucleic acid sequence for 4661 Mut-SIRT |
| SEQ ID NO: 8 | Nucleic acid sequence for 4663 Mut-SIRT |
| SEQ ID NO: 9 | Nucleic acid sequence for 4664 Mut-SIRT |
| SEQ ID NO: 10 | Nucleic acid sequence for 4665 Mut-SIRT |
| SEQ ID NO: 11 | Amino acid sequence for SlbZIP1 SIRT M1L M4L |
| | M10L |
| SEQ ID NO: 12 | Nucleic acid sequence encoding targeting region of |
| | bZIP1 gRNA1 |
| SEQ ID NO: 13 | Nucleic acid sequence encoding targeting region of |
| | bZIP1 gRNA2 |
| SEQ ID NO: 14 | Nucleic acid sequence encoding targeting region of |
| | bZIP1 gRNA3 |
| SEQ ID NO: 15 | Nucleic acid sequence encoding targeting region of |
| | bZIP1 gRNA4 |
| SEQ ID NO: 16 | Nucleic acid sequence encoding targeting region of |
| | bZIP2 gRNA1 |
| SEQ ID NO: 17 | Nucleic acid sequence encoding targeting region of |
| | bZIP2 gRNA2 |
| SEQ ID NO: 18 | Nucleic acid sequence encoding targeting region of |
| | bZIP2 gRNA3 |
| SEQ ID NO: 19 | Nucleic acid sequence encoding targeting region of |
| | bZIP2 gRNA4 |
| SEQ ID NO: 20 | Amino acid sequence for McCpf1 nuclease |
| SEQ ID NO: 21 | Nucleic acid sequence encoding Solanum lycopersicum |
| | bZIP1 |
| SEQ ID NO: 22 | Nucleic acid sequence encoding Solanum lycopersicum |
| | bZIP2 |
| SEQ ID NO: 23 | Nucleic acid fragment from wild-type tomato plant |
| SEQ ID NO: 24 | Nucleic acid fragment from mutated tomato plant with |
| | 8 bp deletion |
| SEQ ID NO: 25 | Nucleic acid fragment from mutated tomato plant with |
| | 10 bp deletion |
| SEQ ID NO: 26 | Nucleic acid fragment from mutated tomato plant with |
| | 9 bp deletion |
| SEQ ID NO: 27 | Nucleic acid sequence encoding Capsicum annuum |
| | bZIP1 |
| SEQ ID NO: 28 | Nucleic acid sequence encoding Capsicum annuum |
| | bZIP2 |
| SEQ ID NO: 29 | Nucleic acid sequence encoding Solanum melongena |
| | bZIP1 |
| SEQ ID NO: 30 | Nucleic acid sequence encoding Solanum melongena |
| | bZIP2 |
| SEQ ID NO: 31 | Amino acid sequence of Capsicum annuum bZIP1 |
| SEQ ID NO: 32 | Amino acid sequence of Capsicum annuum bZIP2 |
| SEQ ID NO: 33 | Amino acid sequence of Solanum melongena bZIP1 |
| SEQ ID NO: 34 | Amino acid sequence of Solanum melongena bZIP2 |
| SEQ ID NO: 35 | Nucleic acid sequence for SIRT element of Capsicum |
| | annuum bZIP1 |
| SEQ ID NO: 36 | Nucleic acid sequence for SIRT element of Capsicum |
| | annuum bZIP2 |
| SEQ ID NO: 37 | Nucleic acid sequence for SIRT element of Solanum |
| | melongena bZIP1 |
| SEQ ID NO: 38 | Nucleic acid sequence for SIRT element of Solanum |
| | melongena bZIP2 |
| SEQ ID NO: 39 | Amino acid sequence for SIRT element of Capsicum |
| | annuum bZIP1 |
| SEQ ID NO: 40 | Amino acid sequence for SIRT element of Capsicum |
| | annuum bZIP2 |
| SEQ ID NO: 41 | Amino acid sequence for SIRT element of Solanum |
| | melongena bZIP1 |
| SEQ ID NO: 42 | Amino acid sequence for SIRT element of Solanum |
| | melongena bZIP2 |
| SEQ ID NO: 43 | Polypeptide fragment from wild-type Arabidopsis |
| | thaliana bZIP1 |
| SEQ ID NO: 44 | Polypeptide fragment from wild-type Solanum |
| | lycopersicum bZIP1 |
| SEQ ID NO: 45 | Polypeptide fragment from wild-type Capsicum |
| | annuum bZIP1 |
| SEQ ID NO: 46 | Polypeptide fragment from wild-type Solanum |
| | melongena bZIP1 |
| SEQ ID NO: 47 | Polypeptide fragment from wild-type Arabidopsis |
| | thaliana bZIP3 |
| SEQ ID NO: 48 | Polypeptide fragment from wild-type Solanum |
| | lycopersicum bZIP2 |
| SEQ ID NO: 49 | Polypeptide fragment from wild-type Capsicum |
| | annuum bZIP2 |
| SEQ ID NO: 50 | Polypeptide fragment from wild-type Solanum |
| | melongena bZIP2 |
| SEQ ID NO: 51 | Polypeptide fragment from wild-type Arabidopsis |
| | thaliana bZIP2 |
| SEQ ID NO: 52 | Polypeptide fragment from wild-type Arabidopsis |
| | thaliana bZIP11 |
| SEQ ID NO: 53 | Polypeptide fragment from wild-type Arabidopsis |
| | thaliana bZIP44 |

1. A plant or plant part comprising altered expression or function of a basic region/leucine zipper motif (bZIP) transcription factor gene or a homolog thereof, wherein said plant or plant part comprises one or more insertions, substitutions, or deletions in an upstream open reading frame (uORF) of the bZIP transcription factor gene or homolog thereof, wherein the uORF comprises a sucrose induced repression of translation (SIRT) element; wherein the one or more insertions, substitutions, or deletions alter function of the SIRT element; and wherein sugar content of said plant or plant part is increased relative to a control plant or plant part.

2. The plant or plant part of claim 1,
(i) wherein the SIRT element within the uORF of the bZIP transcription factor gene or homolog thereof in which the one or more insertions, substitutions, or deletions are located comprises a nucleic acid molecule:
(a) comprising a nucleic acid sequence encoding a polypeptide comprising an amino acid sequence having at least 90% sequence identity to the amino acid sequence set forth in SEQ ID NO: No: 5 or 6, wherein the SIRT element retains SIRT function;
(b) comprising a nucleic acid sequence encoding a polypeptide comprising the amino acid sequence set forth in SEQ ID NO: 5 or 6;
(c) comprising a nucleic acid sequence having at least 90% sequence identity to the nucleic acid sequence set forth in SEQ ID NO: 3 or 4, wherein the SIRT element retains SIRT function; and/or
(d) comprising the nucleic acid sequence set forth in SEQ ID NO: 3 or 4;
(ii) wherein the plant or plant part comprises a deletion of 7-9 nucleotides in the nucleic acid sequence encoding the SIRT element; and/or
(iii) wherein the nucleic acid sequence encoding the SIRT element comprising the deletion:
(a) comprises a nucleic acid sequence that shares at least 90% identity with a nucleic acid sequence selected from the group consisting of SEQ ID NOs: 7-10; or
(b) comprises a nucleic acid sequence selected from the group consisting of SEQ ID NOs: 7-10.

3.-4. (canceled)

5. The plant or plant part of claim 1,
(i) wherein expression of a bZIP transcription factor gene is increased as compared to the control plant or plant part;
(ii) wherein expression or function of a molecule regulated by the bZIP transcription factor is modulated, wherein the modulation comprises:
(a) an increased baseline expression or function in the absence of sucrose relative to the control plant or plant part;
(b) decreased suppression of the expression or the function in the presence of sucrose relative to the control plant or plant part; and/or
(c) an increase in the expression or the function in the presence of sucrose relative to in the absence of sucrose;
(iii) wherein the molecule regulated by the bZIP transcription factor comprises from about 1.1-fold to about 25-fold increase in the baseline expression or function relative to the control plant or plant part;
(iv) wherein the molecule regulated by the bZIP transcription factor comprises from about 1.1-fold to about 10-fold increase in the expression or the function in the presence of sucrose relative to in the absence of sucrose; and/or
(v) wherein the molecule regulated by the bZIP transcription factor is one or more of asparagine synthase (ASN), proline dehydrogenase (PDH), sucrose phosphate synthase (SPS), sucrose-6′-phosphate phosphatase (SPP), and calmodulin.

6.-9. (canceled)

10. The plant or plant part of claim 1, wherein a level of total soluble solids (TSS) is increased in the plant or plant part relative to the control plant or plant part, and/or wherein the sugar is one or more of total sugar, sucrose, glucose, fructose, galactose, maltose, and lactose.

11. (canceled)

12. The plant or plant part of claim 1, wherein the bZIP transcription factor gene encodes an SlbZIP1 transcription factor or an SlbZIP2 transcription factor.

13. The plant or plant part of claim 12, wherein said SlbZIP1 transcription factor: (a) comprises a nucleic acid sequence having at least 80% sequence identity to SEQ ID NO: 21; or (b) comprises the nucleic acid sequence set forth in SEQ ID NO: 21; and
wherein said SlbZIP2 transcription factor: (c) comprises a nucleic acid sequence having at least 80% sequence identity to SEQ ID NO: 22; or (d) comprises the nucleic acid sequence set forth in SEQ ID NO: 22.

14. The plant or plant part of claim 1, wherein the plant or plant part is a tomato plant or plant part.

15. A method for altering expression or function of a basic region/leucine zipper motif (bZIP) transcription factor gene or a homolog thereof in a plant or plant part, said method comprising introducing one or more insertions, substitutions, or deletions into the upstream open reading frame (uORF) of the bZIP transcription factor gene or the homolog thereof in the plant or plant part, wherein the uORF comprises a sucrose induced repression of translation (SIRT) element;
wherein function of the SIRT element is altered; and wherein sugar content of said plant or plant part is increased relative to a control plant or plant part.

16. The method of claim 15,
(i) wherein the SIRT element within the uORF of the bZIP transcription factor gene or homolog thereof to which the one or more insertions, substitutions, or deletions are introduced comprises a nucleic acid molecule:
(a) comprising a nucleic acid sequence encoding a polypeptide comprising an amino acid sequence having at least 90% sequence identity to the amino acid sequence set forth in SEQ ID NO: No: 5 or 6, wherein the SIRT element retains SIRT function;
(b) comprising a nucleic acid sequence encoding a polypeptide comprising the amino acid sequence set forth in SEQ ID NO: 5 or 6;
(c) comprising a nucleic acid sequence having at least 90% sequence identity to the nucleic acid sequence set forth in SEQ ID NO: No: 3 or 4 and encoding a SIRT element, wherein the SIRT element retains SIRT function; and/or
(d) comprising the nucleic acid sequence set forth in SEQ ID NO: 3 or 4;
(ii) wherein the one or more insertions, substitutions, or deletions comprise a deletion of 7-9 nucleotides in the nucleic acid sequence encoding the SIRT element;
(iii) wherein the deletion is introduced into the SIRT element which:
(a) comprises a nucleic acid sequence that shares at least 90% identity with a nucleic acid sequence selected from the group consisting of SEQ ID NOs: 7-10; or
(b) comprises a nucleic acid sequence selected from the group consisting of SEQ ID NOs: 7-10.

17.-18. (canceled)

19. The method of claim 15, comprising introducing a gene editing system into the plant or plant part.

20. The method of claim 19,
(i) wherein the gene editing system is one or more of a zinc finger nuclease system, a transcription activator-like effector nucleases (TALEN) system, and a clustered regularly interspaced short palindromic repeats (CRISPR) system;
(ii) wherein the gene editing system comprises a CRISPR nuclease, a Cas12a nuclease, or McCpf1;
(iii) wherein the gene editing system comprises one or more guide RNAs (gRNAs);
(iv) wherein the one or more gRNAs comprise a nucleic acid sequence complementary to a region of a nucleic acid sequence encoding a SIRT element;
(v) wherein at least one of the gRNAs is encoded by a nucleic acid sequence;
(a) comprising a nucleic acid sequence that shares at least 80% sequence identity with the nucleic acid sequence selected from the group consisting of SEQ ID NOs: 12-19; or
(b) comprising a nucleic acid sequence selected from the group consisting of SEQ ID NOs: 12-19.

21.-26. (canceled)

27. The method of claim 15,
(i) wherein expression or function of the bZIP transcription factor is increased as compared to the control plant or plant part;
(ii) wherein expression or function of a molecule regulated by the bZIP transcription factor is modulated, wherein the modulation comprises:
(a) an increased baseline expression or function in the absence of sucrose relative to the control plant or plant part;
(b) decreased repression of the expression or the function in the presence of sucrose relative to the control plant or plant part; and/or
(c) an increase in the expression or the function in the presence of sucrose relative to in the absence of sucrose;
(iii) wherein the molecule regulated by the bZIP transcription factor comprises from about 1.1-fold to about 25-fold increase in the baseline expression or function relative to the control plant or plant part;
(iv) wherein the molecule regulated by the bZIP transcription factor comprises from about 1.1-fold to about 10-fold increase in the expression or the function in the presence of sucrose relative to in the absence of sucrose; and/or
(v) wherein the molecule is asparagine synthase (ASN), proline dehydrogenase (PDH), sucrose phosphate synthase (SPS), sucrose-6′-phosphate phosphatase (SPP), or calmodulin.

28.-31. (canceled)

32. The method of claim 15, wherein a level of total soluble solids (TSS) is increased in the plant or plant part relative to the control plant or plant part, and/or wherein the sugar is one or more of total sugar, sucrose, glucose, fructose, galactose, maltose, and lactose.

33. (canceled)

34. The method of claim 15, wherein the bZIP transcription factor gene encodes an SlbZIP1 transcription factor or an SlbZIP2 transcription factor.

35. The method of claim 34, wherein said SlbZIP1 transcription factor: (a) comprises a nucleic acid sequence having at least 80% sequence identity to SEQ ID NO: 21; or (b) comprises the nucleic acid sequence set forth in SEQ ID NO: 21; and
wherein said SlbZIP2 transcription factor: (c) comprises a nucleic acid sequence having at least 80% sequence identity to SEQ ID NO: 22; or (d) comprises the nucleic acid sequence set forth in SEQ ID NO: 22.

36. The method of claim 15, wherein the plant or plant part is a tomato plant or plant part.

37. A nucleic acid molecule comprising a nucleic acid sequence encoding a mutated SIRT element, comprising one or more insertions, substitutions, or deletions compared to the corresponding native nucleic acid sequence encoding a SIRT element, wherein said nucleic acid sequence encoding the mutated SIRT element:
(a) comprises a nucleic acid sequence that shares at least 90% identity with a nucleic acid sequence selected from the group consisting of SEQ ID NOs: 7-10; or
(b) comprises a nucleic acid sequence selected from the group consisting of SEQ ID NOs: 7-10.

38. The nucleic acid molecule of claim 37,
(i) wherein said nucleic acid sequence encoding a mutated SIRT element is operably linked to a nucleic acid sequence encoding a bZIP transcription factor;
(ii) wherein the bZIP transcription factor gene encodes an SlbZIP1 transcription factor or an SlbZIP2 transcription factor; and/or
(iii) wherein said SlbZIP1 transcription factor:
(a) comprises a nucleic acid sequence having at least 80% sequence identity to SEQ ID NO: 21; or
(b) comprises the nucleic acid sequence set forth in SEQ ID NO: 21; and wherein said SlbZIP2 transcription factor:
(c) comprises a nucleic acid sequence having at least 80% sequence identity to SEQ ID NO: 22; or
(d) comprises the nucleic acid sequence set forth in SEQ ID NO: 22.

39.-40. (canceled)

41. A DNA construct comprising, in operable linkage:
(a) a promoter that is functional in a plant cell; and
(b) the nucleic acid molecule of claim 37 comprising a nucleic acid sequence encoding a mutated SIRT element.

42. A cell comprising the nucleic acid molecule of claim 37 or the DNA construct of comprising the nucleic acid molecule of claim 37.


SEQUENCE LISTING (53 sequences)

SEQ ID NO: 1 - RNA, 1278 nt, Solanum lycopersicum
  source 1..1278: mol_type = mRNA
aaaccttctttcgcctacccttggttcctcatttgatagattgatttgcgtttgtttgctgaaggcactgtttggatttggatttggatttacacaacaaccattgaatttttactaatttgatatattcgattattgtttcattgattgaggtttgataaatagtttctctatgttagtttcaaattattcatgatccacatgcgtcgagttcgcattatgcactctttctccgttgtcttcctgtactggttctatgtgttttcatgaattagcccacctcaaacccttgaaccctcctcctgtttgatttagaaattagaagtaccaaattcagaaaccgaacgagaaaaaagtagttacctctccttttttgaatccctatttttatcgaacaaacttccccattgaataaacaacacaaagacatggcttcgactcagcaaccagctagttcgggttctgatggccagcgatatgctactaatgatgagagaaaacgaaagagaatggagtctaaccgtgaatctgcaaggcggtcacggaagaggaagcagcagcatttggaggagttgatgagccaattgacacagctgcagaatcagagcactatctggcgtgagaagattgaatctgttggaagaaacttccacacccttgatgcggagaacaatgtcttgagggctcaaatggctgaactgactgaacgcttagattctctgaattccctcactcgtttttgggctgatgctaatggacttgctgtcgacatccctgaaattccagacactttgcttgagccttggcagctcccttgcccaattcaacccattactgcctctgctgacatgtttcagttttgagcacaacctgtgtgtatgattggactaccatgatcctagctctcgggttcttgcttaacaggtgctccttgtatcctcttcgattcaattacatgcaacagctgcagcgaaagacaaccttgttactatttcccgaagttctcctttatgcagtcactgcattttgtttatttgcaatgtgagttagtagcttttcttgtgctctctttgtgtcagtttatgactatagattctctatgtaatatttagcttcatggattagcagtttctagtactttgtcaatgatcttcagtctctagtttataaataaaaatcttaagtcgtgtgaacgatatgacccttttttgtttctgcaagattgctctgatcattcaactagttctgagtaattgtgtttctgcttggcacaatgtt

SEQ ID NO: 2 - RNA, 1211 nt, Solanum lycopersicum
  source 1..1211: mol_type = mRNA
ccatattacacctatgacacaactttccttaaaataattttataaaaaaaaaaactcaaaaagcttcttcttctagcctatataaaccccattctctactcctttataacattctttcaaccttttggttcaacaacttccctctttgcaaagatcatttttgtgaatactttcatttgctgtttggattttggatattctactcaggaaattatttggtttctttgaagactggttggggtctcttacaaagttccaatgtttgattcttagtttcaaaatttcttcatgtcattctctattcgcgtgcgacgagttcggattctgcattctttctcagtagtctttctttactggttctacgtgttttcatgaactagagccctcatcattttcttgttccagtttaatttacaaattccatagccacactatcaaaagtttggtgctttaacaaattatacatagttccctgttattccattttttaaatcattcatccaatctgcctgagagacatgtcttcgactccgcacttggtgagttctggttcagatgcagaccagaggtatgcaaagttcgacgaaaggaaaaggaagagaatggaatccaaccgtgagtcggccaagaggtcacggatgaagaagcagcagcgtttggaggagttaagtagcgaaacaacacagctgcagaaccagaacagtatctgccgtgataagattgcttctgttgaaatgaactatcgttccattgatgcagagaacaacgtgttgagggctcagcttgcagaattgactgaacggttgaattcactgaactcactcacccaattttgggctgatactactggatttcctgtggacattcctgaaattcccgacactttgctggagccatggcaactgccttgcccgattcaacctatcactgcttctgatatgttccagttttgagctctgctggtgtgatgatttacaagaatggaatagagattaagcgtattaagtatctagtttgatattagcaatgtagcttttgtttctgaatagtaatggttggttttggcctattatcgatatctgaatatctagtttgttgtttttagtatttgttcgactttatttagtctgtggatgttatatatgcatcttttggagtactatgaatgagatggcccttctcatttctatgaaaaaattgctgtgtgttga

SEQ ID NO: 3 - DNA, 75 bp, Solanum lycopersicum
  source 1..75: mol_type = genomic DNA
atgatccacatgcgtcgagttcgcattatgcactctttctccgttgtcttcctgtactggttctatgtgttttca

SEQ ID NO: 4 - DNA, 87 bp, Solanum lycopersicum
  source 1..87: mol_type = genomic DNA
atgtcattctctattcgcgtgcgacgagttcggattctgcattctttctcagtagtctttctttactggttctacgtgttttcatga

SEQ ID NO: 5 - protein, 25 aa, Solanum lycopersicum
MIHMRRVRIMHSFSVVFLYWFYVFS

SEQ ID NO: 6 - protein, 28 aa, Solanum lycopersicum
MSFSIRVRRVRILHSFSVVFLYWFYVFS

SEQ ID NO: 7 - DNA, 67 bp, synthetic construct
  source 1..67: mol_type = other DNA
atgatgtcgagttcgcattatgcactctttctccgttgtcttcctgtactggttctatgtgttttca

SEQ ID NO: 8 - DNA, 67 bp, synthetic construct
  source 1..67: mol_type = other DNA
atgatccacatgcgtcgagttcgcattatgcactctttctccgttgtcttccttctatgtgttttca

SEQ ID NO: 9 - DNA, 66 bp, synthetic construct
  source 1..66: mol_type = other DNA
atgatccacatgcgtcgagttcgcattatgcactctttctccgttgtcttcctctatgtgttttca

SEQ ID NO: 10 - DNA, 68 bp, synthetic construct
  source 1..68: mol_type = other DNA
atgatccacatgcgtcgagttcgcattatgcactctttctccgttgtcttcctgtactggttctatgt

SEQ ID NO: 11 - protein, 25 aa, synthetic construct
LIHLRRVRILHSFSVVFLYWFYVFS

SEQ ID NO: 12 - DNA, 24 bp, synthetic construct
  source 1..24: mol_type = other DNA
tctatgttagtttcaaattattca

SEQ ID NO: 13 - DNA, 24 bp, synthetic construct
  source 1..24: mol_type = other DNA
aaattattcatgatccacatgcgt

SEQ ID NO: 14 - DNA, 24 bp, synthetic construct
  source 1..24: mol_type = other DNA
tccgttgtcttcctgtactggttc

SEQ ID NO: 15 - DNA, 24 bp, synthetic construct
  source 1..24: mol_type = other DNA
aggtgggctaattcatgaaaacac

SEQ ID NO: 16 - DNA, 24 bp, synthetic construct
  source 1..24: mol_type = other DNA
aaaatttcttcatgtcattctcta

SEQ ID NO: 17 - DNA, 24 bp, synthetic construct
  source 1..24: mol_type = other DNA
ttcatgtcattctctattcgcgtg

SEQ ID NO: 18 - DNA, 24 bp, synthetic construct
  source 1..24: mol_type = other DNA
tcagtagtctttctttactggttc

SEQ ID NO: 19 - DNA, 24 bp, synthetic construct
  source 1..24: mol_type = other DNA
tttactggttctacgtgttttcat

SEQ ID NO: 20 - protein, 1319 aa, synthetic construct
MALFQDFTHLYPLSKTMRFELKPIGKTLEHIHAKNFLSQDETMADMYQKVKAILDDYHRDFIADMMGEVKLTKLAEFYDVYLKFRKNPKDDGLQKQLKDLQAVLRKEIVKPIGNGGKYKAGYDRLFGAKLFKDGKELGDLAKFVIAQEGESSPKLAHLAHFEKFSTYFTGFHRNRKNMYSDEDKHTAITYRLIHENLPRFIDNLQILATIKQKHSALYDQIINELTASGLDVSLASHLDGYHKLLTQEGITAYNTLLGGISGEAGSRKIQGINELINSHHNQHCHKSERIAKLRPLHKQILSDGMGVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGALYDELAKIPTLYNKVRDYLSQKPFSTEKYKLNFGNPTLLNGWDLNKEKDNFGIILQKDGCYYLALLDKAHKKVFDNAPNTGKNVYQKMIYKLLPGPNKMLPKVFFAKSNLDYYNPSAELLDKYAQGTHKKGNNFNLKDCHALIDFFKAGINKHPEWQHFGFKFSPTSSYQDLSDFYREVEPQGYQVKFVDINADYINELVEQGQLYLFQIYNKDFSPKAHGKPNLHTLYFKALFSKDNLANPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDEADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWKICSHGDKRYVYDKTANQNKGATKGINVNDELKSLFARHHINDKQPNLVMDICQNNDKEFHKSLIYLLKTLLALRYSNASSDEDFILSPVANDEGMFFNSALADDTQPQNADANGAYHIALKGLWVLEQIKNSDDLNKVKLAIDNQTWLNFAQNRKRPAATKKAGQAKKKKGSYPYDVPDYAYPYDVPDYAYPYDVPDYAGSPKKKRKV

SEQ ID NO: 21 - DNA, 435 bp, Solanum lycopersicum
  source 1..435: mol_type = genomic DNA
atggcttcgactcagcaaccagctagttcgggttctgatggccagcgatatgctactaatgatgagagaaaacgaaagagaatggagtctaaccgtgaatctgcaaggcggtcacggaagaggaagcagcagcatttggaggagttgatgagccaattgacacagctgcagaatcagagcactatctggcgtgagaagattgaatctgttggaagaaacttccacacccttgatgcggagaacaatgtcttgagggctcaaatggctgaactgactgaacgcttagattctctgaattccctcactcgtttttgggctgatgctaatggacttgctgtcgacatccctgaaattccagacactttgcttgagccttggcagctcccttgcccaattcaacccattactgcctctgctgacatgtttcagttttga

SEQ ID NO: 22 - DNA, 435 bp, Solanum lycopersicum
  source 1..435: mol_type = genomic DNA
atgtcttcgactccgcacttggtgagttctggttcagatgcagaccagaggtatgcaaagttcgacgaaaggaaaaggaagagaatggaatccaaccgtgagtcggccaagaggtcacggatgaagaagcagcagcgtttggaggagttaagtagcgaaacaacacagctgcagaaccagaacagtatctgccgtgataagattgcttctgttgaaatgaactatcgttccattgatgcagagaacaacgtgttgagggctcagcttgcagaattgactgaacggttgaattcactgaactcactcacccaattttgggctgatactactggatttcctgtggacattcctgaaattcccgacactttgctggagccatggcaactgccttgcccgattcaacctatcactgcttctgatatgttccagttttga

SEQ ID NO: 23 - DNA, 41 bp, synthetic construct
  source 1..41: mol_type = other DNA
ggttctatgtgttttcatgaattagcccacctcaaaccctt

SEQ ID NO: 24 - DNA, 33 bp, synthetic construct
  source 1..33: mol_type = other DNA
ggttctatgtgaattagcccacctcaaaccctt

SEQ ID NO: 25 - DNA, 31 bp, synthetic construct
  source 1..31: mol_type = other DNA
ggttctatgaattagcccacctcaaaccctt

SEQ ID NO: 26 - DNA, 32 bp, synthetic construct
  source 1..32: mol_type = other DNA
ggttctatgtaattagcccacctcaaaccctt

SEQ ID NO: 27 - DNA, 435 bp, Capsicum annuum
  source 1..435: mol_type = genomic DNA
atggcttcagctcagcaaccagctagttcgggctctgatggccagagatatgctaattatgatgagagaaaacgaaagagaatggagtccaaccgtgaatctgcaaggcggtcacggatgaggaaacagcagcatttggaggagttgatgagtgaattgacgcagctgcaaaatcagaacactatttggagccagaggattgatgctgttggaaaaaactatcacacagtcgaagcagagaacaatgtcttgagggctcaaattgctgaactgactgaacgcttggattcccttaattcgctcactcgtttctgggctgatgctaatggacttgctgtggacatccctgaaattccagacgctttgcttgagccctggcagctcccttgcccaattcaacccatcactgcctctgctgatatgttgcagttttga

SEQ ID NO: 28 - DNA, 435 bp, Capsicum annuum
  source 1..435: mol_type = genomic DNA
atgactttgactccacaatccgtgagttctggttcggatgctgatccgaggtatgtaaagtttgatgaaaggaaaatgaagagaatggaatccaaccgccagtcggccaagaggtcacggatgaggaagcagcagcgtttggaggagttaaagagcgaaacaactcagctgcagaaccagaacagtatctgccgccataagattgattctgtcgaaaggaagtatcattccgttgacactgagaacaacgtgttgagggctcaactcgcagaactgactgaacggttgaattcgctgaacgaactcactcaattctgggctgataccactggatttcctgtcgacgttgctgaaattcccgacattttgctggagccgtggcaactgccgtgcccaactcatgctatcgctgcttccgatatgttccaattttga

SEQ ID NO: 29 - DNA, 435 bp, Solanum melongena
  source 1..435: mol_type = genomic DNA
atggctttgactcagcaatcagctagttcgggttccgatggccagcgatgtgctactaatgatgagagaaaacgaaagagaatggagtctaaccgtgaatctgcaaggcggtcacggatgaggaagcagcagcatttggaggagttgatgagccaattgacgcagctgcagagtcagagcactatctgtcgtgagaggattgattctgttggaagaaacttccacatccttgacgcggagaataatgtcttgagggctcaaatagctgaactgactgaacgcttggattcactgaattccctaactcgtttctgggctgatgctaatggacttgctgtggacatccccgaaataccagactctttgctcgagccctggcagctcccttgcccaattcaacccattactgcctctgctgatatgttggagttttga

SEQ ID NO: 30 - DNA, 435 bp, Solanum melongena
  source 1..435: mol_type = genomic DNA
atgtcttcgactccacaattggtgagttctggttcggatgcagaccagaggtatgcgaagtttgatgaaaggaaaaggaagagaatggaatccaaccgtgagtctgccaggaggtcacggatgaggaagcaacagcgtctggaggagttaacgagcgaaacaacacagctgcagaaccagaacagtatctgccgcgataagattgattctgtggagaggaattatcgtgccattgatgcagagaacaatgtgttgagggctcagctcgctgaattgactgaacgtttgaattcactgaactcactcgcccaatgttgctctgatactactggatttcctgtggacattcctgaaatccctgacgcatggctggagccatggcaactgccttgcccgatccaacctatcactgcttccgatatgttccagttttga

SEQ ID NO: 31 - protein, 144 aa, Capsicum annuum
MASAQQPASSGSDGQRYANYDERKRKRMESNRESARRSRMRKQQHLEELMSELTQLQNQNTIWSQRIDAVGKNYHTVEAENNVLRAQIAELTERLDSLNSLTRFWADANGLAVDIPEIPDALLEPWQLPCPIQPITASADMLQF

SEQ ID NO: 32 - protein, 144 aa, Capsicum annuum
MTLTPQSVSSGSDADPRYVKFDERKMKRMESNRQSAKRSRMRKQQRLEELKSETTQLQNQNSICRHKIDSVERKYHSVDTENNVLRAQLAELTERLNSLNELTQFWADTTGFPVDVAEIPDILLEPWQLPCPTHAIAASDMFQF

SEQ ID NO: 33 - protein, 144 aa, Solanum melongena
MALTQQSASSGSDGQRCATNDERKRKRMESNRESARRSRMRKQQHLEELMSQLTQLQSQSTICRERIDSVGRNFHILDAENNVLRAQIAELTERLDSLNSLTRFWADANGLAVDIPEIPDSLLEPWQLPCPIQPITASADMLEF

SEQ ID NO: 34 - protein, 144 aa, Solanum melongena
MSSTPQLVSSGSDADQRYAKFDERKRKRMESNRESARRSRMRKQQRLEELTSETTQLQNQNSICRDKIDSVERNYRAIDAENNVLRAQLAELTERLNSLNSLAQCCSDTTGFPVDIPEIPDAWLEPWQLPCPIQPITASDMFQF

SEQ ID NO: 35 - DNA, 78 bp, Capsicum annuum
  source 1..78: mol_type = genomic DNA
atgatcctcatgcgtcgtattcgcattatgcactctttctccgttgtattcctgtactggttctatgtgttttcatga

SEQ ID NO: 36 - DNA, 87 bp, Capsicum annuum
  source 1..87: mol_type = genomic DNA
atgactttctctatccgtatgcgtagaatccggatccttcactctttctcagtcgtctttctttactggttctatgatttttcatga

SEQ ID NO: 37 - DNA, 78 bp, Solanum melongena
  source 1..78: mol_type = genomic DNA
atgaccctcatgcgtcgaatacgtattatgcattctttctctgtcgtcttcctgtactggttctatgtgttttcatga

SEQ ID NO: 38 - DNA, 87 bp, Solanum melongena
  source 1..87: mol_type = genomic DNA
atgtcattctctacccgtatgcgtcgagttcggattatgcactctttctcagtagtccttcttcactggttctacatgttttcatga

SEQ ID NO: 39 - protein, 25 aa, Capsicum annuum
MILMRRIRIMHSFSVVFLYWFYVFS

SEQ ID NO: 40 - protein, 28 aa, Capsicum annuum
MTFSIRMRRIRILHSFSVVFLYWFYDFS

SEQ ID NO: 41 - protein, 25 aa, Solanum melongena
MTLMRRIRIMHSFSVVFLYWFYVFS

SEQ ID NO: 42 - protein, 28 aa, Solanum melongena
MSFSTRMRRVRIMHSFSVVLLHWFYMFS

SEQ ID NO: 43 - protein, 25 aa, Arabidopsis thaliana
MINLNQFLVYHSISVVILHWFYVIS

SEQ ID NO: 44 - protein, 25 aa, Solanum lycopersicum
MIHMRRVRIMHSFSVVFLYWFYVFS

SEQ ID NO: 45 - protein, 25 aa, Capsicum annuum
MILMRRIRIMHSFSVVFLYWFYVFS

SEQ ID NO: 46 - protein, 25 aa, Solanum melongena
MTLMRRIRIMHSFSVVFLYWFYVFS

SEQ ID NO: 47 - protein, 28 aa, Arabidopsis thaliana
MSYSILFRRIRILHSFSVVYLYYTYVFS

SEQ ID NO: 48 - protein, 28 aa, Solanum lycopersicum
MSFSIRVRRVRILHSFSVVFLYWFYVFS

SEQ ID NO: 49 - protein, 28 aa, Capsicum annuum
MTFSIRMRRIRILHSFSVVFLYWFYDFS

SEQ ID NO: 50 - protein, 28 aa, Solanum melongena
MSFSTRMRRVRIMHSFSVVFLHWFYMFS

SEQ ID NO: 51 - protein, 41 aa, Arabidopsis thaliana
MTPVLCEIFLSGLTVKSALCRRTHLVQSFSVVFLYWFYNVS

SEQ ID NO: 52 - protein, 42 aa, Arabidopsis thaliana
MSPIILSEIFLSGFMLNSGIRRRTHLVQSFSVVFLYWLYYVS

SEQ ID NO: 53 - protein, 41 aa, Arabidopsis thaliana
MSPVISEILRSGLTIDSSLRRRTHLVQSFSVVFLYWFYVFS